SEQUENCE LISTING (49 sequences)

SEQ ID NO: 1 - DNA, 20 bp, synthetic construct
  source 1..20: mol_type = other DNA
gcccacttccactatgtcct

SEQ ID NO: 2 - DNA, 20 bp, synthetic construct
  source 1..20: mol_type = other DNA
gtgagggtgggactgtctac

SEQ ID NO: 3 - DNA, 20 bp, synthetic construct
  source 1..20: mol_type = other DNA
tacgttgtagcccacttcca

SEQ ID NO: 4 - DNA, 20 bp, synthetic construct
  source 1..20: mol_type = other DNA
gggtgggactgtctactgag

SEQ ID NO: 5 - DNA, 21 bp, synthetic construct
  source 1..21: mol_type = other DNA
tgtagcccacttccactatgt

SEQ ID NO: 6 - DNA, 20 bp, synthetic construct
  source 1..20: mol_type = other DNA
aggaggtggagtgttgctag

SEQ ID NO: 7 - DNA, 20 bp, synthetic construct
  source 1..20: mol_type = other DNA
cagtttcatgcccatcgtcc

SEQ ID NO: 8 - DNA, 19 bp, synthetic construct
  source 1..19: mol_type = other DNA
taggctgccaatggtgagg

SEQ ID NO: 9 - DNA, 20 bp, synthetic construct
  source 1..20: mol_type = other DNA
cccctctagagcccactgta

SEQ ID NO: 10 - DNA, 20 bp, synthetic construct
  source 1..20: mol_type = other DNA
taatgctaggctgccaatgg

SEQ ID NO: 11 - DNA, 20 bp, synthetic construct
  source 1..20: mol_type = other DNA
acagtgaaatgccccaacta

SEQ ID NO: 12 - DNA, 20 bp, synthetic construct
  source 1..20: mol_type = other DNA
cagggaggtagcgatgagag

SEQ ID NO: 13 - DNA, 21 bp, synthetic construct
  source 1..21: mol_type = other DNA
acaaactaccacctacctccc

SEQ ID NO: 14 - DNA, 20 bp, synthetic construct
  source 1..20: mol_type = other DNA
cagggaggtagcgatgagag

SEQ ID NO: 15 - DNA, 22 bp, synthetic construct
  source 1..22: mol_type = other DNA
tgccccaactaaatactaccgt

SEQ ID NO: 16 - DNA, 20 bp, synthetic construct
  source 1..20: mol_type = other DNA
gctcaggcgtttgtgtatga

SEQ ID NO: 17 - DNA, 20 bp, synthetic construct
  source 1..20: mol_type = other DNA
cgccgcagtactgatcattc

SEQ ID NO: 18 - DNA, 20 bp, synthetic construct
  source 1..20: mol_type = other DNA
cagggttgattcgggaggat

SEQ ID NO: 19 - DNA, 20 bp, synthetic construct
  source 1..20: mol_type = other DNA
ctccctcaccaaagcccata

SEQ ID NO: 20 - DNA, 21 bp, synthetic construct
  source 1..21: mol_type = other DNA
gggttaggatgagtgggaaga

SEQ ID NO: 21 - DNA, 20 bp, synthetic construct
  source 1..20: mol_type = other DNA
cccacaatcctaggcctacc

SEQ ID NO: 22 - DNA, 20 bp, synthetic construct
  source 1..20: mol_type = other DNA
atgaaggagaggggtcaggg

SEQ ID NO: 23 - DNA, 20 bp, synthetic construct
  source 1..20: mol_type = other DNA
actcacaacaccctaggctc

SEQ ID NO: 24 - DNA, 20 bp, synthetic construct
  source 1..20: mol_type = other DNA
gcttcgacatgggctttagg

SEQ ID NO: 25 - DNA, 20 bp, synthetic construct
  source 1..20: mol_type = other DNA
gctcccttcccctactcatc

SEQ ID NO: 26 - DNA, 20 bp, synthetic construct
  source 1..20: mol_type = other DNA
tgagaatgagtgtgaggcgt

SEQ ID NO: 27 - DNA, 20 bp, synthetic construct
  source 1..20: mol_type = other DNA
ccttggctatcatcacccga

SEQ ID NO: 28 - DNA, 20 bp, synthetic construct
  source 1..20: mol_type = other DNA
tcttgggcagtgagagtgag

SEQ ID NO: 29 - protein, 921 aa, Caenorhabditis elegans
MFNSQSKRAYQQIDDDFDDEVLRNSTCTSRFMQGWGSSTRSLLFGGASNDEQRNLIASSSSHHSYHDSPAEEPPETHYEQFTATHNHGPPTMASSSQLNSRRWDHVLNLDEFFTHIYEYHQNGGYLCIVLQKVFSLLQFIFVMSFTTFFTQCVNYQFLFANTNVTSHGTVNQGKRHFGDAVVDNCPAHISIWMIFAILAAIVYWITRVIKHAQYIMKMSEIQQFYAHELKIADDQLPNLTWHAIVKRICEAQKKLRLSIHQDNITSIYIYHRILRYKNYMTGMINKRILHPVFDVPFLGPIAYLPNNLKHEIERILFTSSTSAWTNGPNLREEYKHHEQLDMAAKKMKEDCKFYGFLSLVLMPLLLPFQIMESFFSLTELIKRRPDGLGMRRYSNFGRYLLRHFNELDHELSARLNRSHIYAAAYMDQFFSPVLEIAAKNITFIAAAVFGVLTILSAWDEDVLQVEHVITVLTICGIVVLVCRGMIPDENLVWQPEILMTHVTSELHYLPSTWKGKAHTTGVRHEFDQLFQMKWMFFVLELTSPIFTPFVLLFWLRPRCSQLANFFHDYTERVDGLGDVCSFAVMDVGKHGDPKWNHIKELKAIVEDQEDQQQAQSVVTSLNRARDGKTELSILHFKTTNPEWQPPKASEKFLRKFRNRLGQEASMLAPLTSMHLGQQMDRQQQQDQILPPRNILLESVHSIVPTTSGGISASQVAPGRHPLIGDGLHRIDGPVGNAFQGIQGAKLGSGGVLASLYQEQPRAAESLSNSLRASGVDIDGAGAEMRINALFLRGLHDESIIHSSSRNYGGTTSSFNMHPTAMQSVFAMPDGFGQPAPAVESSLIDIETPTYHRESVQRSAAEHKMEHETPEPLAELPDLPGPSSSEHQQRSRLVPNTQHRQINEEEEEEEEEDNTPPLSFSS

SEQ ID NO: 30 - DNA, 3588 bp, Caenorhabditis elegans
  source 1..3588: mol_type = genomic DNA
atgttcaactcacagtcaaaacgggcatatcagcaaatcgacgacgattttgatgatgaagtgctgcgtaacagtacgtgtacgtcacgatttatgcaaggatggggctcatccacaaggtcactgctttttggaggcgcttccaatgatgaacagaggaatttaatcgccagctcctcgtcccatcactcgtatcacgattcacctgccgaggagccaccggaaactcactacgaacaattcacggccacccacaatcatggtccgccaacaatggcttcatcgtcacagctaaactctcgacgatgggaccacgtactgaatttagacgaatttttcacgcatatctacgaatatcatcagaatggcggttatttgtgcattgttttgcagaaagtgttcagtttattacaatttatatttgttatgagttttactacattcttcactcaatgcgtcaactatcaattcctattcgccaacacaaacgtgacaagtcacggcactgtaaatcagggaaaacggcatttcggagacgctgtcgtcgataattgcccggcacacattagtatttggatgattttcgcgattctcgccgccatcgtctactggatcactcgggtcatcaaacacgctcagtacattatgaaaatgtcggaaattcagcaattttatgctcacgagctgaaaattgccgacgatcagctgcccaatttgacgtggcacgcgattgtgaagaggatttgtgaggcgcagaaaaaactacggttgagcattcatcaggataatattacgtcgatttatatttatcatcgcattcttcgttataagaattatatgactggaatgattaataagcgaatccttcacccagtcttcgatgttcctttcctcggaccaatcgcctatcttcccaacaatctaaaacacgaaatcgagcgaattctgttcacaagttccacgtcagcatggacgaatggtccgaacctccgagaggagtacaaacatcacgagcaactcgatatggcagccaagaaaatgaaggaagattgcaaattctacggatttctgtcgctcgtcctgatgcctctactgctcccattccagattatggaatcgttcttctcactgacggagctcataaaaagacgaccagatgggcttggaatgagaagatacagtaattttggaagatacctcctgcgtcacttcaacgagcttgaccacgaactctcggctcgcctgaatcgttcacacatttacgcggcagcctacatggatcaattcttctccccagtcctggaaatcgccgccaaaaatatcacattcatcgccgccgccgtcttcggagtgctgacaattctgtctgcgtgggatgaggatgttctccaggtggaacacgtcatcaccgttctcacgatctgcggaatcgtggttctcgtgtgtcgtggaatgattcccgacgagaatctcgtctggcaacccgagatcctcatgacccacgtcacttcggagcttcactacctgccgtccacgtggaagggaaaggcacatacgacaggcgtcaggcacgaattcgatcagcttttccagatgaaatggatgttttttgtgctggaactgacgtcgccgatctttacaccattcgttctgctgttctggttgagacccaggtgctcacagctcgcgaatttcttccatgactatacggaacgagtcgatgggctcggcgatgtgtgagcttcgctgtgatggatgttggaaagcatggtgatccgaagtggaatcatattaaagagctcaaagcgatcgttgaggatcaggaggatcagcagcaggcccagtcggtggtgacatcattgaatcgggctcgcgatggaaaaacggagctatcgattctccatttcaagactaccaatcccgaatggcagccgcctaaggcgtcggagaaatttttgaggaaatttaggaatcggttggggcaggaggcttccatgctcgcaccgctgacctcaatgcatctcgggcagcaaatggatcggcagcagcagcaggatcaaatcctgccaccgcgcaacattctcctcgaatccgtacactcgattgtcccaacaacttccggcggaatttcagcttcccaggtcgcaccgggccgccatccactcatcggagacggccttcaccgtatcgacgggcccgttggaaacgcgttccagggaattcagggagccaagctgggcagcggtggtgtgctcgccagcctctaccaggagcagccacgtgccgccgagtcgctgagcaacagtttgagagctagtggagttgatatcgatggagccggagccgaaatgcgcatcaacgccttgttcctgagaggtcttcacgatgagagtattatccacagctcatcccgaaactacggcggcacgacgtcaagcttcaacatgcacccgaccgctatgcaaagcgttttcgcgatgcccgacggctttggccagccggctcccgccgtagaatcatcccttatcgacattgaaacacccacctatcacagggaatcagtgcagagatcggcggcggagcacaagatggagcacgagactcccgagccactggccgagcttccggatcttccgggaccgtcatcgtcggagcatcagcagagatcccgactcgttccgaacacccagcatcggcagattaatgaggaggaagaagaggaagaggaggaggacaatacgccaccgcttagctttagctcttagattttctgaattaaaaaaaaaaaatttttttaatccctgcattatttcctcgttttcccctaccaattgacgtgaaaacgcgctccgcctccaaaccatgggtctcgttaggtatttggcgcgaaaaccgacaatttgaacgtattcaccaattttcaagcatttaaaattatttttaagtcgaatttcggcaaaaacacgaaaaaccgtttttttttttcgattttcaagcattttttgtatttttaaaaatgatttttacgttgatttttcgcataaaaaccgaaaacatctgaaaattaaaaaaaatcacaaaaaaaaaattaaataattttttccgatttcaacaattttcaagcattttttgtctggtttttatgccattccggttaaaaaaaaaaactctaaaatacgaaaaatgcttgaaaaagtgacaaaaacgattaaaacaagcttttttattgatttttgcgtcgaatttcggcttaattatccataaaaaccgggaaaagctcgaaaattttttaccgtttgcgggggtgggacgttttcaatttgccgtggagcgcgtttacacggttttcacccaatttctaacctgaactgcagcataattttgtacatttttgtgtatgttgtaccgttttctgttgtttttttttgtaaattttgaaactttgacacgagtttacattagtcagaatagtttttttttctatgtatgaacacaattttttttcgaattttttttcaccgcagctccctcaccgctactctgtacaatttttttcactgacaatctgaataaaatcccgcaaatttg

SEQ ID NO: 31 - protein, 221 aa, Caenorhabditis elegans
MSSFLEFAKPKMLDIKRKINFASGEKTDESVQPQQQTEQSSAQQTTPSAKAVSNPFITPLTESTPGMSESWVELAPSRTSLCSSVDINMVIIDEKDKDSRLSPVSIAQSPHVEFESLEQVKYKLVREMLPPGKNTDWIWDWSSRPENTPPKTVRMVQYGSNLTTPPNSPEPELYQYLPCESDSLFNVRVVFGFLVTNIFSFVVGAAVGFAVCRKLIKHHRQ

SEQ ID NO: 32 - DNA, 1168 bp, Caenorhabditis elegans
  source 1..1168: mol_type = genomic DNA
agacgggtctttcctaacaatgtcctcatttcttgagtttgcaaaacccaaaatgctcgatatcaaaagaaagatcaactttgcgtccggcgagaagacggatgagtctgtgcaaccgcaacagcagacggagcagtcatcggctcaacagacaacaccatccgcaaaagccgtctcaaacccatttatcaccccattgaccgagagtacacctggtatgtcagaatcgtgggtggaactggcaccaagccgtaccagtttgtgcagtagcgtcgatattaatatggtgatcattgatgaaaaagataaggattcgaggctgagcccagtctcaatcgcacaatctcctcacgtggaattcgagagcttagaacaagtaaagtacaagttggttagagaaatgcttcctcccggaaagaatactgattggatttgggattggagcagtcggccagaaaatacacctccaaagactgtccgtatggttcaatatggctccaaccttaccactccgccaaactctcccgagccagagctctaccaatacttgccatgcgaatctgattcgcttttcaacgttcgcgtagtctttggtttcttagtcaccaacatcttcagctttgtcgttggagcagcagtaggatttgctgtttgccggaaactaatcaaacatcatcgtcaataattccaaacaaaaattttaacgttccacttacctcatttcactgtacatacattccagcagtgctggagattgggcatttttaccctttgattcacaatactacattctctgcaaaattcttccggagtcttcttgcatcttgtcaaaaagattcgaataaacaataattttgccatgtttcccttctctccaaattgtcatctttcaaactgtctcctgaatattcctaatactcaaaacattcctactacagttttgaattcaacacatctaatcgcgatttgctctcacgattaggtaattttaaattaattatgtacataatatagctttatacactgaatgtaattacgactttttctttgctgttacctgggcattttgattctgaaacttgcttttccatgtttttctgtcatcacaaattgtaaatatttctaaaatactgttacaaaaccacgcattttaataaaatgtaaatat

SEQ ID NO: 33 - protein, 138 aa, Caenorhabditis elegans
MVDLSKNDGGSGKAGKGVSDAIDTVLYYVVDLKKQQPMVQLGVGAGFGTVTGYFVTKGGRLVAATVGISFLLAQFAIHKGYITLNESKIERDMKNLHKSVMNKVSGKKVINISDSFVSEYRWILGGFAAGMLIGFSVA

SEQ ID NO: 34 - DNA, 584 bp, Caenorhabditis elegans
  source 1..584: mol_type = genomic DNA
ccagccatggttgatctttcaaaaaacgacggaggcagtggaaaagccggaaaaggtgtgagtgatgccatcgatacagtcctctactacgttgttgatctgaaaaagcagcagccgatggtccagctgggtgttggtgctggattcgggacagtgactggatattttgtcaccaagggaggacgtcttgttgctgccacagtgggaatctctttcctgttggctcaattcgctattcacaaaggatacatcacgctgaacgaatcaaaaatcgaacgagacatgaaaaatctacacaagtccgtcatgaacaaagtatccggcaagaaagttatcaacatttccgattcatttgtctccgaataccggtggattcttggcggattcgcagctgggatgctgattggatttagcgtcgcctaaatgtggaaaattgtgaagcttcagttggtttttatctacgatctgtactatttataatcgagttgctcgatatgatgcataaataactttttctcactctccattgtctagtcatgctgtctatttttttcttcacattgactgataaattatacaccctt

SEQ ID NO: 35 - protein, 579 aa, Caenorhabditis elegans
MNTGNPDTRRNNQVPMGVVNLLSRSSFPSVDSYLALSVLVAIVASVTVFTTFRSQPELQKLIEEELRNNTRLSSAYGLNIEALSGHTFFQIAHYILSDTTLIWVAINSYFAILAVCTRLIIKLTFKELARQEENVARQAFFCYVLLTIVYLSVVIGPQKGHRVMPWMIWGGICAFLSHLQFITCQRLKHISPSCDRGSQKISFLSLFLFFVSIAMTFLISRFQHHLTWQPAVLLYFDCLLAVFRSTYILFRCISSSRVFSFNPDSVRHFNYWLELITNFVCELIQMLSFAQLLAFSPGLNLTSIFFLYHMKLTYNCMTEQLSRHRNHKKIFEHIERSYPSVKCANGDDRCVVCWELLGTSRRLPCSHQFHDWCLMWWLAQDSSCPTCRCTIPSPQDQIRQPPEVGNSTRLRFNGGSFGFVHFPAFTLEVAANFGPFFGRAAEPTEEQLQTMLEQVREMFPQMSVDIIMTDLRQSGSAQSTIENILEGRIGMNASFMPGGVLDDELSDESENEIEYEEPAEIVQEPDNGRQRTWTKLSSSSGDEDLSYYEIQRAKMIETYRRKYLESDKAADLRAMGITE

SEQ ID NO: 36 - DNA, 2188 bp, Caenorhabditis elegans
  source 1..2188: mol_type = genomic DNA
atgaatacggggaacccggatacccgtcgtaacaatcaggtacccatgggggttgtcaatctactatcccgatcatcatttccatctgtcgactcatatctggcattgtcagttcttgttgcaattgttgcttctgtcactgtatttacaacatttcgctcacaacccgagctacagaagcttatcgaagaagaacttcggaataatacacgactgtcatcagcatatggtttgaatattgaagcattgtccgggcacacattctttcaaattgctcattacattttatctgatacaacgctaatctgggttgctataaactcgtattttgccatactagcagtttgtacgagactaataatcaaattaacattcaaagagctcgcccgacaggaggaaaatgtggcccgtcaagctttcttctgttatgtcctgttaacaatcgtctacttatcagttgtgatcggaccgcaaaaggggcatcgtgtaatgccatggatgatctggggtggtatttgtgctttcttatcccatttgcagtttatcacatgtcagaggctcaagcatatctctccatcctgtgatcgtggaagtcagaagatttcattcctctcgttgttcctcttcttcgtttcgattgccatgacgttcttgatttctcgattccaacatcacttaacatggcaacctgctgtacttttatattttgattgccttctcgcagtattccgttccacttacattctgttccgatgcatttcttcatctcgtgtattctccttcaatcctgactctgttcgtcatttcaactattggctcgagcttatcaccaactttgtctgcgaactcattcaaatgctcagttttgctcaacttttggcattctcacctggacttaatctcacaagcatcttcttcttgtatcacatgaagcttacctacaactgcatgactgaacaattaagtcgtcatcgtaatcataagaagatcttcgaacacattgagagatcatatccaagtgttaaatgtgctaatggtgatgatcgttgcgttgtttgctgggaattgctcggaacatctcgaagacttccatgctctcatcaattccatgattggtgtctcatgtggtggcttgctcaggattcttcatgtccaacgtgtcgttgtactattccatctccacaagatcaaatcagacaaccaccagaggttggaaatagtacaagattacgtttcaatggtggatcatttggattcgttcatttcccagcattcactcttgaagtagctgctaacttcgggcctttctttggtcgagcagccgaaccaacagaagagcaattgcaaacaatgctcgagcaagtcagggagatgttcccgcagatgtctgttgatataataatgacggatctacgacaatcgggatccgctcagtctacaatcgaaaatattctagaaggaaggataggaatgaatgcatcatttatgcccggtggggttttggatgatgaactctcagatgagagtgaaaacgaaatagaatatgaagagcccgccgaaatagtccaagaaccagataatggccgtcaaagaacgtggacaaaattgagttcttcatcaggagatgaagatctatcatactatgaaatccagcgagccaaaatgatcgaaacttaccgacggaaatatctggagtctgataaagctgccgatctacgagcaatgggaatcaccgaataattcatactcatttctctcctcttttctgtttacttaggatgttcttgtcatttcaaagctacctggtagcgtactctcattatttctaaattcatgcctgtatgagtatagttttttgttgtatattttgtatccatttgatgagattctctgagattcccatctttcatcatatttagcatagttccaccctctttttcctttcctttcatgaccctcttttcatgtatgggtatgatcatcatccacaccaactatcaattttctctctgtatcctctatcgagattctgaatctcgttctcttgttccttttttccgtgtttcctctgaaatctcgactatcattctaatcctttacaattctcatcccggtaccttgtgagtgtgaacctgtacattttttctccgatccccatttttcaccaaagtaaatactttgagttttc

SEQ ID NO: 37 - protein, 259 aa, Caenorhabditis elegans
MHYYILPILFSIYIIATCNLCEDSMITCPDLKDQCDQGFVQSQCPYTCGICHQDPNQCTDTNLDCLKFDSMCKTDVKYQKQCPYTCGTCVPATPDSCFDHMIECPNYSVPCTDAVKIQCPKRCGVCTSGASNSTLAPPRPSPTPPCFDSGNECATYTLPCDVNQKIFCPRTCGVCGSTGVPMAQTTLLTTVKPTTTVVPSTTTKLPATTTKKLPSTTPKPPCKDSSPNCAGWAKNGFCTNTFYPPEKRKEYCAKTCRMC

SEQ ID NO: 38 - DNA, 955 bp, Caenorhabditis elegans
  source 1..955: mol_type = genomic DNA
acttttaaacaaatatcatcattattttaatcaccatcatcatcatcttggtttctaacccccatcttaactgataacacactgaaacttttgttataaaacaaggcattacatcaagtattatgtactagattttcccacaaaaaatgcactactacatcctgcctattcttttttcaatatacattattgcaacttgcaatctatgtgaagactcaatgatcacatgtccggatctcaaggatcaatgtgatcagggatttgtgcagagtcaatgcccatacacttgtggaatatgccatcaggatccaaatcaatgtactgacactaacttagattgcttgaagtttgattcaatgtgcaagacggatgtgaagtatcagaaacaatgcccatacacctgtggaacctgcgtaccagctaccccggacagctgctttgatcatatgattgagtgcccaaattatagcgttccatgtactgacgctgttaagatacaatgcccaaaaaggtgtggagtttgcacctcgggagccagtaattcgacattagctcctcccagaccgtcaccaactccaccatgttttgatagtggcaacgaatgcgccacctatacgctgccatgtgatgtgaaccaaaagatattctgcccacggacctgtggagtttgtggaagtactggggtacctatggctcaaactacgttgctcactacggtaaagcctacgactacagtagtaccaagtactacaacaaaactgccagctaccacgaccaagaagcttccatcaactacgccaaagccaccatgcaaggattcgtcgccaaactgtgccgggtgggcaaaaaacggcttctgcacgaacaccttctatccaccggagaagagaaaggagtactgtgcaaagacatgtagaatgtgctgattttttgatttaataaattatttattctt

SEQ ID NO: 39 - protein, 386 aa, Caenorhabditis elegans
MSDEISILIQDRKTGQRRNLTLNINITGNIEDLTKDVEKLTEIPSDELEVVFCGKKLSKSTIMRDLSLTPATQIMLLRPKFNSHNENGATTAKITTDSSILGSFYVWCKNCDDVKRGKLRVYCQKCSSTSVLVKSEPQNWSDVLKSKRIPAVCEECCTPGLFAEFKFKCLACNDPAAALTHVRGNWQMTECCVCDGKEKVIFDLGCNHITCQFCFRDYLLSQLERFGFVNQPPHGFTIFCPYPGCNRVVQDVHHFHIMGQTSYSEYQRKATERLIAVDDKGVTCPNVSCGQSFFWEPYDDDGRSQCPDCFFSFCRKCFERNCVCQSEDDLTRTTIDATTRRCPKCHVATERNGGCAHIHCTSCGMDWCFKCKTEWKEECQWDHWFN

SEQ ID NO: 40 - DNA, 1266 bp, Caenorhabditis elegans
  source 1..1266: mol_type = genomic DNA
ctctcatgaagcatgtctgatgaaatctctatattaatacaagatagaaaaacaggtcaacgtaggaatctaacacttaatataaatataactggaaatatcgaagatctcacaaaagatgtggaaaagctcaccgaaattcccagcgatgagctggaagtggttttctgtgggaaaaagttatcaaaatcaacgattatgagggatttgtcactgacacctgcaacacaaatcatgcttctccgtccaaagttcaatagtcacaacgaaaacggtgctactactgcaaaaataacaacagattcttcaattctcggaagcttctacgtgtggtgcaaaaattgtgacgacgtcaagcgcggcaaactgcgggtttattgccaaaaatgctcgtcaacctctgttctagtcaaatctgaaccccagaactggtccgacgttctcaaaagcaagagaataccggcggtctgcgaagaatgctgtactccaggtcttttcgctgaattcaagttcaaatgtctagcctgcaacgatccggccgcagctctaactcacgtacgcggaaattggcaaatgaccgagtgctgtgtttgtgatgggaaggagaaagtgatcttcgacctcggatgcaatcatattacatgccaattctgtttcagagattatttgctaagtcaactggaacgattcggttttgtcaatcagccgccgcatggcttcaccattttctgcccctatccagggtgcaatagagtggtacaagatgtgcaccatttccacattatgggtcagacgtcgtacagcgaataccaacggaaagccaccgagcgattgattgccgtggacgacaagggtgtgacttgcccgaatgtctcgtgtgggcagagcttcttctgggagccctatgatgacgatggaagatcccagtgtccagattgttttttttcgttttgcagaaagtgcttcgaaagaaattgtgtgtgccagagcgaagacgatctcacccgaactacaattgacgcgactacaagaagatgcccaaaatgccacgtggcaaccgaacggaacggcggatgtgctcacattcactgtacctcgtgtggaatggattggtgtttcaagtgcaagacagaatggaaggaagagtgtcaatgggaccattggtttaattaataactctctaaaattcatcttttctagagattgtacgtgttattctgtagatttttacaacaattacggtgtgtctgaagttacattgacatt

SEQ ID NO: 41 - protein, 641 aa, Caenorhabditis elegans
MSMKRFGKAAYRIANELVAKGGRLPIFQRFLPRIFPATYNLGVHVVLKKAPFPRQNALRIARLVTRHGRVFRPFSSVIIERHRFQNQNDWRRKFQPIRKELPRNVDLVERIRQIFGNSLRYNEDLKSTEWPNRIDSYEFGEFLGQGCNAAVYSARLANSDAESSGNTHYGAGFNEVTNILAEIPPVSKVAQKKFPLAIKLMFNFEHDRDGDAHLLKSMGNELAPYPNAAKLLNGQMGTFRPLPAKHPNVVRIQTAFIDSLKVLPDAIERYPDALHTARWYESIASEPKTMYVVMRRYRQTLHEYVWTRHRNYWTGRVIIAQLLEACTYLHKHKVAQRDMKSDNILLEYDFDDEIPQLVVADFGCALACDNWQVDYESDEVSLGGNAKTKAPEIATAVPGKNVKVNFEMADTWAAGGLSYEVLTRSNPFYKLLDTATYQESELPALPSRVNFVARDVIFDLLKRDPNERVKPNIAANALNLSLFRMGEDVKQMMEKCGISQMTTLLAGSSKVLSQKINSRLDKVMNLITAETIMANLAPHLISRAERQLRATFLSRMNREDIWRSLQYFFPAGVQLDTPATSSDCLETISSLMSSFSNDSENYEKQQKPAKNGYNNVPLLLRNVIRTDADGINGIVHRVRSK

SEQ ID NO: 42 - DNA, 2115 bp, Caenorhabditis elegans
  source 1..2115: mol_type = genomic DNA
tgtatagaagtaagaatgtctatgaaacgattcggaaaagcagcatatcgaatcgcaaatgagttagttgcaaaaggtggacgactaccaattttccaacgcttcctgccgagaatatttcccgccacttataatttaggagttcatgtcgtactcaaaaaggctccatttccacgacaaaatgctctacgaattgctcgccttgtaactcgccacggtcgagttttccggccattttcctcagtaataatcgaaagacatcgatttcaaaatcaaaatgattggcgtcgaaagtttcaaccgattcgtaaagaattgccaagaaatgtggatttagtcgaacgaatcaggcagatatttggcaattctctacgatacaatgaggatttgaaaagcactgaatggccgaatagaattgattcttatgagtttggggaatttctcggtcaaggatgcaatgcagcagtttactctgcgagattagccaattctgatgcagaatcctcagggaatactcactatggtgcagggtttaatgaagtcacaaatatacttgcagaaattccgccagttagcaaagttgcacaaaagaaattcccgttggcaatcaaattaatgtttaattttgaacatgatcgcgatggagatgctcatctcttgaaatcaatgggaaatgaattggctccatatccgaatgctgcaaagttgctcaatggacaaatgggaacatttagacctcttccagcaaaacatccaaatgttgttcgaattcagacagcttttattgattcgttaaaagttttgccagatgcgattgaacgatatccagatgcccttcacactgcacgttggtatgagtcaattgcctccgaaccgaaaacaatgtacgtagtaatgagacgataccgacaaacacttcatgaatatgtatggactcgtcatcgaaattattggacaggacgagtgataattgctcaactattagaagcatgtacatatcttcataagcataaagttgctcagcgagacatgaaaagtgataatattcttctggaatatgattttgacgacgagattccccaattagttgtcgccgattttggatgtgcacttgcatgtgacaattggcaagtagactatgaatcagatgaagttagtcttggaggaaatgccaagacaaaagcaccagaaattgcgacggctgttcctggaaagaatgtaaaagtaaacttcgaaatggcagatacatgggcagctggaggcctttcttatgaagttctaacacgatcaaatccattctacaaacttcttgatactgcaacataccaggaatcagaactaccagcactcccatctcgtgtcaattttgtggcacgagatgtcatttttgacctactcaagcgagatcctaatgaaagagtcaagccgaatattgctgcaaatgcgttgaatttgtcattgttcagaatgggagaagatgtgaagcagatgatggaaaaatgtggaatatctcaaatgactactctattggctggaagttctaaagttttgagtcaaaaaatcaatagtcgtctggacaaagtgatgaatctgattactgctgaaactatcatggccaacctagctccacatttgattagtcgagcagaacgacaacttcgagcaacatttctttcaagaatgaatcgagaagatatttggagaagtcttcaatatttcttcccagctggtgttcaacttgacacacctgccacatcatcagactgtttggagactatttccagtttgatgtcgagtttttcaaatgattcagaaaattacgagaagcaacagaaaccggctaaaaatggatacaacaatgttccacttcttctcagaaatgttatccgtacagatgcggatggaatcaatggaattgtacatagagttcgatctaaatagatttgttttaacgattactgtagtttgaaacctctagattacatatgtaataaccttttttttttacttgttggtatttttcaaattgtacaatcacttttcatgccagtctattgcgttcttttgtctaattacggtttatcattccctcatttatggaaaatatcatacctt

SEQ ID NO: 43 - DNA, 7263 bp, Caenorhabditis elegans
  source 1..7263: mol_type = genomic DNA
agactgctccgcgacggtgaaactcttgaagatctccggcgtctgtccccagaagaaattctcatgagatgggttaattatcatttggaacgtgctggaactcaacgtcgcctacacaatttcacatcagatattgtcgattcagagatttatactcatttgctccatcaaattgctccaaatggctccggtgtcacgttatctccactcggagtccatggaaatgtcccaagagccggagcaatgttagatgaagccgagaagcttgattgtcgggaatttgtgacagcaactgatgtggcagccggaaattacaaattaaacttggcttttgttgccaatttgttcaataaacatccaaatttgccggatccgggagcggatgaagttggtgagtttttgaaaaagtcgaaaattgactgaaaaataaaaaatttcaaaatttcaaaatgacaacttccagcgattttaattaaaaaaggtccgataaaaaaataaaaaataagatttttcatcgaaaattcaccgactcaccgaagtcgactagaattctgaaaatgacgatttttagccaaaaatttcaaattttttcatttttttggtgaaaaatttgaaatttttggctaaaaatcgtcattttcagaattttaggcgactttcggtgaattttcgatgaaaaatcttattttttagactgataatcgccattttttgagaaaaatcggaaattttgaacaaaaatcattttctcattttcattttcagaattttaagcgaatttcagcgaaaaaatcggaccttttttaattaaaatcgctggaagttgtcattttcaaattttaagcgatttttggcgaaaaatttgaaatttctggccgaaaatcgtctttttcagaattttaggggcgacttttggcgaaaaatctaaaatatttaactgaaaaccactaaaacttgatcaattttaacctttttacttaaaatctctaaaaatcgattaattttcgattttttgatgaaaagttcgacattttttactgaaaatcaattaaattttaaattttcagcgaaaaaatggagtttcttgtagaaaatcgttaaaaattgatcaatttttcaaatttttacactaaaaatcgtcattttcaaaattacaggcaattttcgacaaaaaaataaagctaaaaattcgatttttaataaaaaatgaattttttttgctggaaatcgactttttcagattttcaaacgatttttgaagaaaaattcggagtttttgacttcaaatcgctaaaaattggccaattgtcgatatttaatgaaaaatctgaattttttttgctggaaatcgctcaaattctgaatttgcagcgaaaaaattgcattttttgttgaaaatcgctaaaaattgaataatttgcgtttttttttttggtgaaaaatcttaatttttgactgaaaattgtctggattcaactgtaaatttgtgaaaaactggaaaaatcgaaaaaattgatttttacatgaaaatcttgaatttttccgtaagttgtgtattgaaacacgaaaaatttcaatttttctccatattgcagctaaaaatctgaaaattttctaaaaatcaaattttatgaattttcagccaattttctggcaaaaattgaaatttttgagtaaaaaattacttcaaaaagatgaaaaattgacttctttttcgaaaaattcaatagaaatttgaatttttaaaacacgaaaaataccaaaattccccacatttccgaacagtttctaatcgtttttcggaggtttttcagtggaagacgtcatcgaagagacacgtgaagagaaaacatatagaaattggatgaattcaatgggtgttgatccatatgttaattggttgtatgctgatcttcaaaatggtgtcgtcattttccaggtaaaaattggatgaaaaccccgagttttccggcgaaaaatggacaaaattccggaaaaaccgattttcccggcggaaatttgacaaaaatcgaaaaaaatctaattttccggcggaagtttaacaaaattccggataaatcgatatttccggcggaaaatcaataaaaatccggaaaaattgcattttccggcgaaaaatggacaaaaatattcagaaaaattgcaaaagttgagtttttcgaggtattttagctcaaaacggatttatctaaattttttactgaaatttcgcgaaattttgacttttttgaagatttttcacattttttgtgaaaaatctgcaaattattcgatttttgctctgaaaatttcagtttttgagttttccaagaatttcagccgaaaaacggattttttcggtttttctgaaatttctgtggaaaatcaaaattttcaagacctttgtgtaatttttcagcccgcatttttaccgagaaatcggcggaagtttaacaaaattccggaaaaatcgatatttccggcggaaaatcaatggaaatccggaaaaattgcattttccggcgaaaaatggacaaaagttcaggaaaaatcaattttgccctgcgaaaaatcaataaattcagaaaaacctcattttaacggcgaaatttcggcaaaattcggtgtgaatttgccctaaaaaaagatctaaaacttgccaattttttcggaaaatccgtatttttgtaccaaaaattcaaatttcacagttacagcatgtgcgcttcattgagaaaatcactcaaaattcaaattttgaagattttctcaatggggcgcactttcaatcgatgcgatgtttaaattttttaaattttcagtcaaaaaaaaatgccaattttgcgagtttttgcagaaaattcgcagtttttccgcatttttcgctataattttcaaaaaaagaatttcagctttacgatataatccgaccgggaatggtaacatggaaacgagttgtacggacattccataagcttcgaggaatgatggatcaaattcagaattgcaattatgctgttgaacttggaaaacagctcagattctcacttgtcggaattcagggaaaaggttgatacttaaaattttttggaatttttacggaaaattatgacaaaaatttacgaaacccatgaaaatttggctgatttttggtctgaaaatgctgaaatcggcagatttttgactaaagaatttgctgaaaacggagaattcttttttttttgttgaaaatcgctaaaaatttaataatttccgttttttttggtgaaaaatcttaattttttgactgaaaattgtctggattgatcaactgtaaatttgtgaaaaactggaaaaatcgaaaaaaaattgatttttaaatgaaaatcttgaatttttccgtaagttctgtattaaaacacgaaaaatttcaatttttctccattttgcagctaaaaatctgaaaattttctaaaaatcaaattttatagtagatacttaaaattttttggaatttttacggaaaattatgacaaaaattcgaaacccatgaaaatttggctgatttttggtctaaaatgctgaaatcggcagatttttgactaaagaatttgctgaaaacggagaattatattcgctttataaatttttattttccgaaataaaaatcagcaaatcgaaattttcatacaacccgccgactcaaaaaaaaacacaaaaatttatggttttcatttaaattttccaacaaaaaaaaacccaaaaaaatctaattttccacacaaaagttccctaaaaaccttccaaactctaatgttatcacaaaaacccaaaaacttgggtgaaattcattttcattcagattttcctttttttaaattcctaaaaacctaatttttcacgcaaaaaactagacattttccaaaaaatattaattttccccccgaaaatctaaaaagttcccaaaaaattcaaatttccaccctaaaatccaaaaatttccatcctaaaaccaaaatttttccaaaaaaaaaaatcgtatttttctccaaatttacccaatttttcttggaaaacagctcagattctcacttgtcggaattcagggaaaaggtagagagaaattcttcagaaataccaagaatttggctgaaaactggacaaattttgagatttttggcgtaactaactgtaaaaagcgtgaaattttgaaagagaaatctggaaaaaatcataaaaatgattaggaaattactgaaaattataattttttcaaattgagatagatattttttgctgaaataacgaaaaatatgtgcttttattgaaaaaaaataccaaaaaaaacatgaagatatggaattttccaaaaaaaaaaaaatccactttttcagagaaaaaattgaatatttttacaaatttcacccaaaaaaccgataaaatgccataaaaaagagcacaattttcaattttaggaaatttcatatttttgaaataaaaaatcgcttaaaaatcctttttttctgaaaatgctcaaaattcgcaaaagagctcagcttgctccaaattatgctcgaaaaaacagaaaaatctcagaaatttgctacgaaaagctctgaaaacttcaaaattgccaaaattgccgaaaatgggccaaaattttgatgagaacgaacttttttgacaaaaaaaaatttgtccaaaaaatcgcatttttctccaaatttaccaacatttttcgttacagatatctacgacggcaatcaaacattgacattagctctcgtttggcagctaatgcgtgcctataccctctcagttcttgctcaatgtactcaatctggtgattccctgccagctgataaggatattgttgcatgggttaacgaaaagctcaaaaattctggaaaatccacatcaattcgatcgttccaagacccggcaatttccgatggaaaagttgtattggatctgattgatgcaattaaaccaaacgttattgatcatagtttggtgaaaagtggcaaaagtaatgaggataaaatgtcaaatgcaaaatatgcaatcacttgtggacgaaagattggagcaaagatttacgcgttgccagaggatattgttgaggttaatattaccccgaaaaattgaagaaaaacggagaaaaatgactgaaaatttgaaattttcagttgaaatttaatcaaaaacgtggaaattttaagaaaaatgactgaaaatcatgaaaaatcaatgatttcggaatttgaagcaaaaaaattccagaagttggagattttcggctaaaatctcactaaaaacttgaaaaattggagaaaaacggagaaaaatgactgaaaattcgagattttggatggaaatttgtttttttcccggcaaatacggctaattttcaattctggcaatttgctgatttgccggatttttttttggcaaattggcgatttgccggtaatatttattttctggcagattgcccatttgccgggaatttttattttctgacaaattgccggtttgccggaaatttttatttcctggcaaattgccgatttgccggaaatttttattctctagcaaattggcgatttgccggaaatatttattttctggcagattgcccatttgccgggaatttttattttctgacaaattgcccatttgccggaaatttttatttcctggcaaattgccggtttgccggaaatatttattttctggcagattggcgatttgccgggaatttttattttctgagaaattgccgatttgacggaaattcttattttctgagaaattgcccatttgccgggaatttttattttctgacaaattgccgatttgccggaaatttttatatcaaaatgattgtaaaacacataatttccagaaaatgtttgaaaaatctacaattttgctctaaaatctcaaaaaaaaatgctccgaaaagctctgaaaatcgtccaaaaccccaaattgttttcaggtgaagccaaaaatggtgttgacagtgtttgcatgtctaatggctcgtgactatcttcccgatatgaagcaaggagcagcttctgctccaattgttccgatgattaatggaaattagttctgcaaaaatttcgaatttcttgaatttcccgccgaaaaaaaatcgcggaaatacaaattttcctccatatatatatctctattttcccgtttttccatcttttttctcacaaaaattgcgtcccgtaagctttaaatgtgatatattcaatttctccccactcattgccacgtggcaaatcacttttttccaattttttttcactttaattgctcatttgctcctccgtttgttactgatttttttctattgcatttaacccctcccgtatttttttcgtgtatttctactaattttcgccttttttctcggatttttggtggaaaattggcggttttttttgaatattttttacccgaaaaatcgagtttctagcctaaatttggaaaaaattgagtttttcaaatttttggaccctttttacgctgaaaaattgcaaaagttgagtttttcgaggtattttagctcaaaacggatttatctaaattttttactgaaatttcgcgaaattttgacttttttgaagatttttcacatttttggtgaaaaatctgcaaatttttcgatttttgctcttaaaatttcagtttttgagttttccaagaatttcagccaaaaaacggattttttcggtttttctgaaatttctctggaaaatcaaaatttctttcaagacctttctgtaatttttcagcccgcatttttaccgagaaatcggcaatttttccgatatttcaaatcgaaatttgaataattatcggatttttttactccaaaaatctaattcggattttggtagaaatttcgaaaaaatcccaaaaaaaatattttttaaaaaaatcccgaaaactcgccgctttttctcccaaaatccgaaatctaacactctgtgaacaactcgtcaaaaattcaaaattatctcttctaatctctcttatctcactcttctcatttttccagttatttgaccaatttccccattgctgctgagcttcttcaatccgagccggcaccccccaacaaattcactgtgaattcccgaccggttgttcgaagcttttcccaaccttcctgttatccatattattattattattcagatgaattttccaaaaattttaatagtttttcggcttttgcgctgccaataaaatttcaaa

SEQ ID NO: 44 - DNA, 530 bp, Caenorhabditis elegans
  source 1..530: mol_type = genomic DNA
gcgacggtgaaactcttgaagatctccggcgtctgtccccagaagaaattctcatgagatgggttaattatcatttggaacgtgctggaactcaacgtcgcctacacaatttcacatcagatattgtcgattcagagatttatactcatttgctccatcaaattgctccaaatggctccggtgtcacgttatctccactcggagtccatggaaatgtcccaagagccggagcaatgttagatgaagccgagaagcttgattgtcgggaatttgtgacagcaactgatgtggcagccggaaattacaaattaaacttggcttttgttgccaatttgttcaataaacatccaaatttgccggatccgggagcggatgaagttggtgagtttttgaaaaagtcgaaaattgactgaaaaataaaaaatttcaaaatttcaaaatgacaacttccagcgattttaattaaaaaaggtccgataaaaaaataaaaaataagatttttcatcgaaaattcaccgactcaccgaagt

SEQ ID NO: 45 - DNA, 509 bp, Caenorhabditis elegans
  source 1..509: mol_type = genomic DNA
atgccaattttgcgagtttttgcagaaaattcgcagtttttccgcatttttcgctataattttcaaaaaaagaatttcagctttacgatataatccgaccgggaatggtaacatggaaacgagttgtacggacattccataagcttcgaggaatgatggatcaaattcagaattgcaattatgctgttgaacttggaaaacagctcagattctcacttgtcggaattcagggaaaaggttgatacttaaaattttttggaatttttacggaaaattatgacaaaaatttacgaaacccatgaaaatttggctgatttttggtctgaaaatgctgaaatcggcagatttttgactaaagaatttgctgaaaacggagaattcttttttttttgttgaaaatcgctaaaaatttaataatttccgttttttttggtgaaaaatcttaattttttgactgaaaattgtctggattgatcaactgtaaatttgtgaaaaactggaaaaatcga

SEQ ID NO: 46 - protein, 419 aa, Caenorhabditis elegans
MSNRNGGGGGGDYQDVIESLRRTQLIFEEDDNNSSDSAPQANHLSRHQTSNSLVEDMVNHSNGNPPPVPPGITQQQCQIGLTPRMSASPPSAVSTISGTAVLGKTMARVQSNPPGSIPHNTTTTAQGIQSVAPHIPIGGGGATDDSSAEILSVFECPVCLEYMLPPYMQCSSGHLVCSNCRPKLQCCPTCRGPTPSVRNLGLEKIANTVRFPCKFSTSGCPLNFHHADKTEHEELCEFRPYCCPCPGASCKWQGGLSDVMEHLKKIHKSITTLQGEDIVFLATDINLPGAVDWVMMQSCFDYNFMLVLEKQEKYDPAQPTQMFYAVVQLIGSKKEADNFVYRLELSASRRRMSWEATPRSIHEGVVVAIQQSDCLAFDSNAAQLFAENGNLGINVTISRIDTPQRRHPNELENSDLEYD

SEQ ID NO: 47 - DNA, 1487 bp, Caenorhabditis elegans
  source 1..1487: mol_type = genomic DNA
agacgacgggaagacggcggcatccgcagacaccgaaccacccaccacctccaacatcgtcgtcctcgtcatctcgactgccttcgctgatgagtaatcggaacggtggtggaggaggaggagactatcaggacgtgatcgagtctcttcgtcgtacacagctcatctttgaggaggacgataataatagtagcgattcggctccgcaagcgaatcatctatccagacatcagactagcaattctttagtcgaagatatggttaatcactcaaatggcaaccctccaccggtcccgccaggcatcacacaacagcaatgtcaaatcggattgacacctcggatgtcggcatcgccaccgtctgcagtgtcaacaatctctggaaccgcagtgcttggcaaaacgatggccagagttcagagtaatcccccgggaagtataccgcacaacacaacgacgaccgcgcaaggaatccaaagcgtcgcaccacatattccaatcggcggcggtggagccaccgacgacagcagtgcagagatcttgtcggtgtttgagtgcccggtatgcctggagtacatgctgccaccatacatgcagtgctcatccggacatcttgtctgctcgaactgcagaccaaagcttcagtgctgcccgacttgtcggggtccaactccttctgtccgcaacctgggcctcgagaagatcgccaacaccgtccgctttccatgcaaattctctacgtccggctgccccctgaacttccatcacgctgataagaccgagcacgaggagctctgcgaattccgaccctactgctgcccgtgccctggagctagctgcaagtggcagggaggcttgtcggatgtgatggagcatttgaagaagatccacaagagcatcacgacgcttcaaggcgaggatatcgtcttcctggccactgacatcaatcttcctggagcagttgactgggttatgatgcagagctgctttgactataattttatgttggtgctcgagaagcaggaaaagtacgatccagctcagccaactcagatgttctacgcagtcgttcagctgattggctcgaagaaggaagctgataactttgtgtaccggcttgagctatcggcgagccgccggagaatgtcatgggaagccacaccgagaagcatccacgagggggtggtagtggccattcaacagtcggattgcctagcgttcgattcaaatgctgcacagctttttgcagaaaacggaaaccttggaatcaacgtcactattagccgaatagatactccacagaggcgccacccaaatgagttggaaaattctgatctcgagtacgactagatatgcacagtgtaaacgtaataaaattatttttgatttttttcttcaaaaatgtgatgaaaagcttaaaaaattcagttgaaaatccccttacaatcagaacattttaaaaaatttaaaaattaaaaaaattttttt

SEQ ID NO: 48 - protein, 856 aa, Caenorhabditis elegans
MEQFDGFEYSKRDLLGHGAFAIVYRGRYVDRTDVPVAIKAIAKKNISKSKNLLTKEIKILKELSSLKHENLVGLLKCTETPTHVYLVMEFCNGGDLADYLQQKTTLNEDTIQHFVVQIAHALEAINKKGIVHRDLKPQNILLCNNSRTQNPHFTDIVIKLADFGFARFLNDGVMAATLCGSPMYMAPEVIMSMQYDAKADLWSIGTILFQCLTGKAPFVAQTPPQLKAYYEKTRELRPNIPEWCSPNLRDLLLRLLKRNAKDRISFEDFFNHPFLTSPLLPSPSKRILESARSPLLANRRIITPQSSLPVPKRAGSTKLDSPTPVRRIGESPRVQRRVITPGMPSPVPGAPMQESTDFTFLPPRQESSPVKQVQVHTNVSPSLTTCKPVPVPSQRLTYQKMEERLAAARKTAVPSSSSPTGSAVSAQHQHQHQQQQEPASSPVVQRIERPDQLPRRTTLQDPNAHDIERMTMPNPTFVVCGSSTKPSPNNANRVRRSTITSPADTQDMVAADQMLSNLDPTTTTTTIPKSATTANIQGIPRGARDRSVTSPPQPTIHENEPLDNAKYQQTDVNNSPTAPTEPFIIKNQTTCSTSSTSSSVVEEEEAMSLPFASGSHLAAGFKKTPAEVPMDHGALPPALDQEIVLGEEHKQILAKLRFVAELVDTLIHVAEQKDNPLASAMASRRQLLTTGTSTTNTSSPYRRAEQLVVYVRALHMLSSALLLAQTNVANRVLHPSVAVQQVLNQLNDKYHQCLVRSQELASLGLPGQDPAMAVISAERIMYRHAIELCQAAALDELFGNPQLCSQRYQTAYMMLHTLAEQVNCDQDKTVLTRYKVAVEKRLRILERQGFVAAVNT

SEQ ID NO: 49 - DNA, 2897 bp, Caenorhabditis elegans
  source 1..2897: mol_type = genomic DNA
aacctacctacaaaaccccccgccaaaacctatatagtcacacaataatcacccaacccagctgaatggagcagtttgacggcttcgagtacagcaaacgggaccttttaggtcatggagcatttgcaattgtatacagaggacgctatgttgatcgcacagacgtgccagttgccatcaaggcgatcgccaagaagaatatcagcaaatcaaagaatctgctgacaaaagagattaaaattctaaaagaattgtcaagcctgaagcatgaaaatcttgtgggcctactcaaatgcacggagacccctactcatgtgtatttggttatggaattctgtaatggaggagatttggctgattatttacaacagaagactacattgaatgaggatactattcaacattttgtcgtgcaaattgctcacgcgttggaagcgatcaacaaaaagggcatcgtacatcgtgatttgaagccccaaaacatcctcctctgcaacaactcccgcacccaaaatccccacttcaccgacatcgtcatcaagcttgccgactttggattcgcccgattcctcaacgacggtgtgatggcggcaactctctgcggatccccaatgtacatggctcccgaagtcataatgtcaatgcaatacgacgcgaaagccgatctctggtcaattggaaccatcctcttccagtgtctcaccggaaaagccccattcgtcgcgcagaccccgccgcagttgaaggcctattacgagaagactcgcgagctgagacccaatattccagagtggtgctctccgaatctacgggatcttctgttgagactgctcaagagaaacgccaaggatcggatctcgttcgaagacttcttcaatcacccgttcctcacctcacctctccttccgtcgccgtcgaagagaatcctcgagagcgcccgatcaccactactggcgaatcgccgaatcatcacaccacaatcttcgcttccagtacccaagcgagccggatccaccaagctcgacagcccgacgccagttcggaggatcggagagtcaccacgtgtacagagaagagtcatcacacccggaatgccgtccccagtgccgggagctccaatgcaagagtccactgatttcactttccttccaccaaggcaggaatccagtccggttaagcaggtccaagtgcataccaatgtctcaccatcactaacaacttgcaagccggtcccagtgccgagtcaacgtttgacctaccagaaaatggaggagcgtctggcggctgcaagaaagactgcagtgccttcttcttcttctccaacaggatcagcggtttctgctcagcatcagcatcagcatcagcagcagcaggagcccgcgtcgtcgccggtggttcagcggattgaaaggcccgatcagctgccaagacggactacgcttcaggatccgaatgctcatgacattgagaggatgactatgccgaatccgaccttcgtcgtatgtggcagcagcacaaaaccatcgccgaacaacgcgaatcgcgttcgccgctccaccatcacaagccccgccgacacccaggatatggtcgctgctgaccaaatgctttcgaacctagaccccaccaccaccaccaccaccatccccaaatcggccaccacagccaacatccagggaattccacgtggcgctcgcgatcgatcggtcacttcgccgccgcagccgacgattcatgagaatgagccgttggataatgcaaagtatcagcagacggatgttaataattcaccgacagcacccacggagccatttattattaagaatcagacaacatgctccacatcctcaacctcctcctcagtcgtcgaagaggaagaggcgatgagtcttccattcgcgtctggaagccatctcgcagcaggattcaagaagacccccgccgaggttccaatggatcacggagccctaccaccagctttggatcaggaaattgtgctcggcgaggagcacaagcagattctcgcgaagcttcgatttgtcgccgagctcgtcgatacccttatccacgtggccgagcagaaggacaatccgttggcgtcggcgatggcttcaaggagacagcttttgactactggaacctcaacgactaatacatcgtcaccgtaccggagagccgagcagctggtggtgtacgtcagagctctgcacatgctctcctcagcactgctcttggctcagactaacgtggcaaatcgggtacttcatccgagtgtcgccgtgcagcaggtgctcaatcagctcaacgacaagtatcatcagtgtttggtccgctcccaggagcttgcctcgcttggcctccccggacaggatccggcgatggcggttatctcggcggaaagaatcatgtacagacatgcgatagaactgtgccaggctgctgctcttgacgagctttttggaaacccccagctgtgcagtcagaggtaccagaccgcctacatgatgcttcacaccttggccgagcaggtcaattgcgatcaggataagacggtgctcacgaggtacaaagtcgctgtggaaaaacgtcttcgaattctggaacgacagggatttgtggcagctgtgaacacataatagtccttttacccccacttttgttctgttttttgtgctgcttcttcccccatttctaccccgccaaaatgagcctctcccttgcaatgaacgtgtagccccccactctattgaaaaatgagattttcactaccctaaacttcttagaattcttttttctattgttttaatgagattttttttttgaaaagacctgtaaatcccgttccggaactagtcgaacataatgcatcattcgtgaataaattttgttgtacagtt

(19) United States
(12) Patent Application Publication
(10) Pub. No.: US 2025/0345296 A1
(43) Pub. Date: Nov. 13, 2025

(54) COMPOSITIONS AND METHODS FOR THE MODULATION OF MITOPHAGY FOR USE IN TREATMENT OF MITOCHONDRIAL DISEASE

(71) Applicant: THE CHILDREN'S HOSPITAL OF PHILADELPHIA, Philadelphia, PA (US)

(72) Inventors: Marni J. FALK, Voorhees, NJ (US); Suraiya HAROON, Philadelphia, PA (US)

(73) Assignee: THE CHILDREN'S HOSPITAL OF PHILADELPHIA, Philadelphia, PA (US)

(21) Appl. No.: 18/871,587

(22) PCT Filed: Jun. 8, 2023
(86) PCT No.: PCT/US2023/068153

§ 371 Date: Dec. 4, 2024

Related U.S. Application Data
(60) Provisional application No. 63/350,188, filed on Jun. 8, 2022.

(51) Int. Cl. A61K 31/167; A01K 67/64; A61K 31/05; A61K 31/444; A61K 31/51; A61K 31/555; A61K 31/7088; C12N 9/22; C12N 15/113; G01N 33/50

(52) U.S. Cl. CPC A61K 31/167; A01K 67/64; A61K 31/05; A61K 31/444; A61K 31/51; A61K 31/555; A61K 31/7088; C12N 9/222; C12N 15/113; G01N 33/5079; C12N 2310/20; G01N 2500/10

(57) ABSTRACT

Compositions and methods for modulation by upregulation (up modulation) and/or downregulation (down modulation) of mitophagy are described for the treatment of mitochondrial disorders including OPA-1 related disease and Single Large Scale Mitochondrial DNA Deletion (SLSMD). Also disclosed are a number of screening assays and gene targets having utility for the identification of agents which modulate the phenotype associated with such disorders.

### CROSS REFERENCE TO RELATED APPLICATION

This application claims priority to U.S. Provisional Patent Application No. 63/350,188 filed Jun. 8, 2022, which is incorporated herein by reference in its entirety.

### GRANT SUPPORT STATEMENT

This invention was made with government support under grant number R35-GM134863 awarded by the National Institutes of Health. The government has certain rights in the invention.

### INCORPORATION-BY-REFERENCE OF MATERIAL SUBMITTED IN ELECTRONIC FORM

The Contents of the electronic sequence listing (CHOP-138-PCT.xml; Size: 72,652 bytes; and Date of Creation: Jun. 8, 2023) is herein incorporated by reference in its entirety.

### FIELD OF THE INVENTION

This invention relates to the fields of physiology and mitochondrial disease. More specifically, the invention provides compositions and methods effective to develop therapeutics and for amelioration of symptoms of mitochondrial disease in human subjects. Also provided are screening methods for identifying novel and potent therapeutic agents and effective protocols for the treatment of mitochondrial disease.

### BACKGROUND OF THE INVENTION

Several publications and patent documents are cited throughout the specification in order to describe the state of the art to which this invention pertains. Each of these citations is incorporated herein by reference as though set forth in full.

Mitochondria are specialized organelles that carry their own genome encoding 22 transfer RNA (tRNA), 2 ribosomal RNA (rRNA) and 13 protein products that participate in the production of cellular energy in the form of ATP. Since the gene products are essential for energy production, maintaining the integrity of the mitochondrial genome (mtDNA) is essential for human health. In fact, aberrations in some or all copies of a cell or tissues' mitochondrial genome can lead to mitochondrial dysfunction, and often result in a wide variety of neuromuscular, multi-system, and metabolic disorders. At the cellular level, a common adaptation to mitochondrial dysfunction is upregulating mitophagy, a mechanism by which dysfunctional mitochondria are tagged and targeted for degradation via the autophagic machinery. During this process, the impact that mitophagy has on the maintenance of mtDNA integrity and the overall health of the cell remains unclear and may depend in part on the specific cause and site(s) of mitochondrial dysfunction.

### SUMMARY OF THE INVENTION

In accordance with the present invention, a composition which modulates at least one of mitophagy, mitochondrial stress level, and mtDNA heteroplasmy level having efficacy for the treatment of mitochondrial disease, comprising effective amounts of one or more agents selected from the agents listed in Table 1 in a pharmaceutically acceptable formulation is provided. In certain embodiments, the composition increases mitophagy or decreases mitophagy. In other embodiments, the composition increases mitochondrial stress level or decreases mitochondrial stress level. In yet another embodiment, the composition increases mtDNA heteroplasmy level, or decreases mtDNA heteroplasmy level. In a preferred embodiment, at least two agents are present and are administered separately. Agents can also be administered together, provided that one does not counteract the beneficial therapeutic effects of the other. The composition can comprise agents selected from at least two of folinic acid, lithium chloride, metformin, N-acetylcysteine, nicotinamide, resveratrol, valproic acid, dexamethasone, etoposide, vorinostat, quercitin, hydralazine, thiamine, lipoic acid, hemin, tripterin, pfithrin-alpha, ginsenoside, sulfonsuccinimidyl olcate, carnitine, AICAR, GSK2578215A, an inhibitory nucleic acid and an activating genetic construct targeting a mitophagy modulator protein encoding nucleic acid in a pharmaceutically acceptable carrier. In certain embodiments, the at least two agents are selected from one or more of vorinostat, hemin, GSK2578215A, tripterin and resveratrol. In another embodiment, the at least two agents are a combination of hemin and tripterin or a combination of thiamine and tripterin, each of these two combinations acting synergistically to modulate one or more of mitophagy, mitochondrial stress level, and mtDNA heteroplasmy level and are administered separately or together. In a preferred embodiment, the disease is OPA-1 disease, Single Large-Scale Mitochondrial DNA Deletion (SLSMD) diseases, or disorders of mitochondrial genome integrity.

Also provided is a method for alleviating symptoms associated with aberrant mitophagy associated mitochondrial diseases, comprising administration of an effective amount of the composition described herein to a patient in need thereof. Symptoms can include without limitation, muscle weakness, exercise intolerance, chronic fatigue, gastrointestinal dysmotility, impaired balance, peripheral neuropathy, metabolic strokes, dysautonomia, vision loss, eye muscle and eyelid weakness, hearing loss, glomerular or tubular renal disease, endocrine dysfunction, diabetes mellitus, dyslipidemia, cardiomyopathy, arrhythmia, anemia, failure to thrive, over or underweight, developmental delay, neurodevelopmental regression, cognitive decline and memory impairment, migraines, headaches, Parkinsonism, dystonia, liver dysfunction or failure, infertility, and metabolic instability.

In certain embodiments the mitochondrial disease is selected from the group consisting of Complex I disease, Complex II disease, Complex III disease, Complex IV disease, Complex V disease, Multiple respiratory chain complex disease, adenine nucleotide translocase deficiency, pyruvate dehydrogenase deficiency, mitochondrial depletion disease, multiple mitochondrial DNA deletions disease, mitochondrial DNA maintenance defects, mitochondrial translation defects, mitochondrial nucleotide import disease, mitophagy disorders, Friedreich's ataxia, Leber's Hereditary Optic Neuropathy, Kearns-Sayre Syndrome, Pearson Syndrome, Chronic Progressive External Ophthalmoplegia, Autosomal Dominant Optic Atrophy, Mitochondrial Myopathy, Mitochondrial Encephalomyopathy with Lactic Acidosis and Stroke-Like Episodes, Myoclonic Epilepsy and Ragged Red Fibers Syndrome, Neurogenic Ataxia and Retinitis Pigmentosa, Mitochondrial Neuro-Gastrointestinal Encephalomopathy, maternally inherited diabetes and deafness, primary lactic acidosis, Leigh syndrome, Leigh-like syndrome, and multi-system mitochondrial disease.

The compositions can include inhibitory nucleic acids which reduce expression of one or more nucleic acids encoding a mitophagy modulator protein selected from atg-9, dct-1, pink-1, sqst-1, hrdl-1, mul-1, pdr-1, plastin-1, siah-1, unc-51, and uaDf5 or agents which increase expression of a mitophagy modulator protein selected from atg-9, dct-1, pink-1, sqst-1, hrdl-1, mul-1, pdr-1, plastin-1, siah-1, unc-51, and uaDf5.

Also provided is a screening method for identifying agents which modulate mitophagy. An exemplary method comprises providing genetically altered C. elegans, said genetic alteration impacting a gene associated with mitophagy, and wild-type C. elegans, lacking said genetic alteration; contacting the C. elegans with an agent; determining whether said agent alters a cellular parameter associated with mitophagy pathway activity(s) in C. elegans comprising said genetic alteration relative to wild-type C. elegans; agents which alter said parameter in said genetically altered C. elegans being identified as modulators of mitophagy. In certain embodiments the cellular parameter is selected from the group consisting of fecundity, egg hatching rate, development, lifespan, stressor survival, healthspan, animal activity, swimming capacity, thrashing activity, pharyngeal pumping rate, mitochondrial oxidant burden, cellular oxidant burden, antioxidant capacity, glutathione levels, reduced (GSH) to oxidized (GSSG) glutathione ratio, CI enzyme activity, CI enzyme assembly, CII enzyme activity, CIII enzyme activity, CIV enzyme activity, complex V enzyme activity, oxygen consumption capacity, ATP production, ATP levels, nicotinamide adenine dinucleotide (NADH and NAD⁺) levels, (NADH and NAD⁺) ratio, NAD metabolism, mitochondrial membrane potential, mitochondrial content, mitochondrial structure, mitochondrial ultrastructure, mitochondrial unfolded protein response, mitochondrial import, mitophagy, autophagy, cytosolic translation activity, nutrient-sensing signaling profile, unfolded protein response activation, lysosomal number, lysosomal activity, lysosomal pH, proteasome number or activity, transcriptome-wide signaling, transcription factor signaling, kinase signaling, amino acid pathway profiles, intermediary metabolic flux dynamics or rates, steady state metabolism of intermediary metabolites, amino acid levels, organic acid levels, ammonia levels, and glycoprotein production, cellular proliferation, cell growth, lactic acid level, glycolysis, cellular redox levels, and lactate/pyruvate ratio. In preferred embodiments, the C. elegans comprise a mutation in a gene that modulates one or more of mitochondrial structure, content, biogenesis, proliferation, destruction, and function. In other embodiments, the C. elegans is genetically altered via introduction of a silencing RNA or antisense oligonucleotide that reduces expression of a gene that modulates mitophagy. Such mutations can be introduced by a system which includes but is not limited to a CRISPR-CAS system, a base editor system or a TAL effector or TALEN system. In certain approaches of the screening method, mitophagy is assessed in live cells harboring an IR161 reporter plasmid in real-time using alterations in green to red fluorescence.

The method described above can further comprise contacting a zebrafish comprising the mutation in the cognate zebrafish gene with said identified agent and determining whether said agent alters a cellular parameter associated with aberrant mitophagy pathway activity in said zebrafish. The method can also comprise contacting a human fibroblast, lymphoblastoid cell line, myoblast cell line, myotube cell line, transmitochondrial cybrid cell line, gastrointestinal cell line, conjunctival derived cell line, cancer cell line, HEK293 cells, HELA cells, derived iPSC or a differentially terminated cell line comprising a mutation in the cognate human gene with said identified agent and determining whether said agent alters a cellular parameter associated with aberrant mitophagy in said human fibroblast or other cell line type.

In certain embodiments, the cells are contacted with a stressor prior before, after, or concomitantly with said agent. In other embodiments, the gene encodes OPA1 mitochondrial dynamin like GTPase or the mutation is a SLSMD that causes SLSMD syndromes (SLSMDS) or a point mutation in mitochondrial DNA that causes a primary mitochondrial disease that may affect any organ function.

Also disclosed is a screening method for identifying agents which modulate mitophagy in zebrafish. An exemplary method comprises providing genetically altered zebrafish, said genetic alteration impacting a gene associated with mitophagy modulation, and wild-type zebrafish, lacking said genetic alteration; contacting the zebrafish from step a) with an agent; determining whether said agent alters a cellular parameter associated with mitophagy modulation in zebrafish comprising said genetic alteration relative to wild type zebrafish; agents which alter said parameter in said genetically altered zebrafish being identified as modulators of mitophagy. The cellular parameter can include without limitation fecundity, egg laying or fertilization rate, development, lifespan, stressor survival, healthspan, animal activity, swimming capacity, vision, hearing, brain death, heartbeat, heart rate, mitochondrial oxidant burden, cellular oxidant burden, antioxidant capacity, glutathione levels, reduced (GSH) to oxidized (GSSG) glutathione ratio, CI enzyme activity, CI enzyme assembly, CII enzyme activity, CIII enzyme activity, CIV enzyme activity, complex V enzyme activity, oxygen consumption capacity, ATP production, ATP levels, nicotinamide adenine dinucleotide (NADH and NAD⁺) levels, (NADH and NAD⁺) ratio, NAD metabolism, mitochondrial membrane potential, mitochondrial content, mitochondrial structure, mitochondrial ultrastructure, mitochondrial unfolded protein response, mitochondrial import, mitophagy, autophagy, cytosolic translation activity, nutrient-sensing signaling profile, unfolded protein response activation, lysosomal number, lysosomal activity, lysosomal pH, proteasome number or activity, transcriptome-wide signaling, transcription factor signaling, kinase signaling, amino acid pathway profiles, intermediary metabolic flux dynamics or rates, steady state metabolism of intermediary metabolites, amino acid levels, organic acid levels, ammonia levels, and glycoprotein production, cellular proliferation, cell growth, lactic acid level, glycolysis, cellular redox levels, and altered lactate/pyruvate ratio.

As above, the genetic alteration can be introduced by a system such as CRISPR-CAS system, a base editor system or a TAL effector or Talen system. The system is useful to introduce a mutation which inhibits expression of a gene selected from atg-9, dct-1, pink-1, sqst-1, hrdl-1, mul-1, pdr-1, plastin-1, siah-1, unc-51, opal, and uaDf5, or a mutation which increases expression of atg-9, dct-1, pink-1, sqst-1, hrdl-1, mul-1, pdr-1, plastin-1, siah-1, unc-51, opal, and uaDf5.

In certain aspects of the method the zebrafish are contacted with a stressor prior before, after, or concomitantly with said agent. Agents identified as having activity in zebrafish can be further screen in C. elegans and human cells.

A preclinical method for identifying mitochondrial disease subjects likely to respond to treatment for aberrant mitophagy is also disclosed herein. An exemplary method comprises contacting patient cell lines or cells obtained from a subject with at least one agent which modulates mitophagy, said subject having a predetermined genotype; culturing said cells under normal and stressed growth conditions, wherein said stressor is applied in increasing concentrations; and determining the protective effects of said agent on said cells, agents having protective action being effective to modulate mitophagy in subjects having said predetermined genotype. In certain aspects the agent activates mitophagy. In other aspects, the agent reduces mitophagy. The method can further include assessing the tolerability and efficacy of said agent in a whole animal model of mitochondrial disease associated with aberrant levels of mitophagy.

Also provided is a method for the treatment of a subject having OPA1 mitochondrial disease, comprising administration of an effective amount of an inhibitory nucleic acid which reduces one or more nucleic acids encoding a mitophagy modulator protein selected from atg-9, dct-1, pink-1, sqst-1, hrdl-1, mul-1, pdr-1, plastin-1, siah-1, unc-51, and uaDf5 in a subject in need thereof, said reduction of said mitophagy modulator protein alleviating one or more symptoms of OPA1 mitochondrial disease.

In another embodiment, a method for the treatment of SLSMD syndrome is provided comprising administration of an effective amount of a compound which increases expression of a mitophagy modulator protein selected from atg-9, dct-1, pink-1, sqst-1, hrdl-1, mul-1, pdr-1, plastin-1, siah-1, unc-51, and uaDf5 in a subject in need thereof, said increase of said mitophagy modulator protein alleviating one or more symptoms of SLSMD mitochondrial disease.

The invention also provides a vector encoding an RNAi vector targeting a plastin gene, wherein said RNAi has a sequence selected from plastin 1A and, or plastin 1B set forth herein below.

In yet another aspect, the invention discloses a humanized C. elegans strain expressing a mutated OPA1 mitophagy modulator protein, said mutation being selected from eat-3(R289Q) or eat-3(V328I).

### BRIEF DESCRIPTION OF THE DRAWINGS

FIGS. 1A-1E: Testing efficacy of mitophagy inhibitors. FIG. 1A) Live mitophagy assessment using mtRosella in C. elegans worms using IR161 reporter plasmid and schematic diagram showing measurement of mitophagy. FIG. 1B) Fluorescent micrographs of C. elegans showing degradation of mitochondria. FIGS. 1A and 1B are prior art. FIG. 1C) Use of IR161 for assessing efficacy of mitophagy modulators. A comparison of fluorescent signals between untreated worms and worms treated with 15 μM Carbonyl cyanide-p-trifluoromethoxyphenylhydrazone (FCCP) for 4-5 hours is shown, where FCCP uncouples the mitochondrial oxidative phosphorphylation (OXPHOS) pathway within the respiratory chain, leading to induction of mitophagy FIG. 1D) A graphical representation of the results shown in FIG. 1C. FIG. 1E) Live mitophagy assessment using mtRosella in C. elegans. Image of mt Rosella reporter animals under basal conditions are greener than the image of the mtRosella animals treated with mitophagy inducing conditions of 4-hour FCCP treatment. Graph shows the average of z-stack images of 3 animals per condition (each data point), where a lower green to red ratio in FCCP treated animals demonstrates that the mitophagy induction reporter functions as expected.

FIG. 2: Inhibition of pink-1 using feeding RNA interference (RNAi) reduces mitochondrial stress in eat-3 (OPA1 homologue) C. elegans knockdown model. Wildtype animals were treated with different feeding RNAi constructs to knock down genes of interest. Knockdown with eat-3 is the disease model (analogous to the known human OPA1 disease (autosomal dominant optic atrophy, ADOA, or ADOA plus) in this case. Pink-1 is a mitophagy pathway gene and pink-/knockdown in the context of eat-3 knockdown rescues the mitochondrial stress (hsp-6p fluorescence conveys induction of the mitochondrial unfolded protein response, or UPR^{mt} pathway) as assessed by fluorescence quantitation.

FIGS. 3A-3C: RNAi targeting 8 different mitophagy modulators reduces mitochondrial stress in eat-3 knockdown C. elegans model. FIG. 3A) KD with gas-1 (complex I subunit, NDUFS2 homologue, also known as K09A9.5 gene in C. elegans) and eat-3 (OPA1 homologue) RNAi induces mitochondrial stress (UPR^{mt}) quantified at the level of hsp-6p fluorescence but knockdown in wildtype worm background with 8 different mitophagy pathway genes (sqst-1, agt-9, pink-1, siah-1, fundc-1, unc-51, dct-1, mul-1) does not. FIG. 3B) Half-dose of eat-3 RNAi induces an equivalent degree of mitochondrial stress as does full-dose eat-3 RNAi treatment. FIG. 3C) Knockdown of same 8 different mitophagy modulator genes as detailed in FIG. 3A legend with RNAi reduces mitochondrial stress in eat-3 RNAi treated wildtype animals. Enhanced reduction of mitochondrial stress obtained with certain combinations of RNAi is shown.

FIGS. 4A-4C: Screening assays assessing the effects of mitophagy modulating agents on animal thrashing behavior in eat-3(R289Q) animals. The R289Q animal was created with CRISPR/Cas9 gene editing technology to model a known OPA1 human patient disease gene allele. FIG. 4A) Folinic Acid, lithium chloride (LiCl), Metformin, N-acetylcysteine (NAC), Nicotinamide, and Resveratrol rescue the eat-3 R289Q animals' thrashing behavior defect. FIG. 4B) Valproic Acid rescues the eat-3 R289Q animals' thrashing defect. Dexamethasone, Etoposide, Vorinostat, and Quercetin are lipophilic compounds known to modulate mitophagy that also modulate animal thrashing behavior in the eat-3 R289Q worms. FIG. 4C) The effects of valproic acid to rescue animal thrashing behavior in the eat-3 R289Q worms are statistically significant in a screen of water soluble compounds that modulate mitophagy.

FIGS. 5A-5B: Screening assay assessing the effects of solid media drug exposure on mitochondrial stress in eat-3(R289Q) animals. FIG. 5A) Hydralazine and nicotinamide significantly rescue mitochondrial stress in eat-3(R289Q) animals. FIG. 5B) 25 mM Thiamine, and 50 mM Thiamine rescue mitochondrial stress in eat-3(R289Q) animals. Treatment with compounds occurred from embryo to L4+1 day adult stage in FIGS. 5A-5B.

FIGS. 6A-6B: Screening assay assessing the effects of solid media drug exposure on mitochondrial stress in eat-3(V328I) animals. The V328I animal was created with CRISPR/Cas9 gene editing technology to model a known OPA1 human patient disease gene allele. FIG. 6A) Folinic Acid and Glucose significantly rescue mitochondrial stress in eat-3 (V328I) animals. FIG. 6B) 25 mM Thiamine, 50 mM Thiamine, 0.2 mM Hydralazine, 0.5 mM Hydralazine significantly rescue mitochondrial stress in eat-3(V328I) animals. Treatment with compounds occurred from embryo to L4+1 day adult stage in FIGS. 6A-6B.

FIGS. 7A-7E: Screening mitophagy modulating compound library of 62 compounds from Medchemexpress assaying mitochondrial stress (hsp-6p::GFP) in eat-3(R289Q) animals. FIG. 7A) Hemin and Tripterin, FIG. 7B) Resveratrol, Pfithrin-α, Ginsenoside, Olanzapine, Dexamethasone, Sulfosuccinimidyl oleate, and FIG. 7C) Resveratrol analog rescues mitochondrial stress in eat-3(R289Q) animals at 25 μM. FIG. 7D) Of the top hits (#10 Hemin, #17 Tripterin, #23 Resveratrol, #35 Sulfosuccinimidyl oleate, #47 Resveratrol analog) tested with a range of doses (12.5 μM, 25 μM, 50 μM and 100 μM), only Hemin and Tripterin rescued the mitochondrial stress response in a dose-dependent manner, FIG. 7E) Three biological replicate testing Hemin and Tripterin in a dose range shows reproducible dose-dependent reduction of mitochondrial stress response.

FIG. 8: Summary table of the significant results on eat-3 animal thrashing behavior and mitochondrial stress induction shown in FIGS. 4-7.

FIG. 9A-9B: Hemin, Tripterin, Thiamine increases ATP production in OPA1 patient cell lines. FIG. 9A) 5 μM Hemin (#10), 10 nM and 50 nM Tripterin (#17), and 5 μM and 50 μM Thiamine treatment increases (not significantly but as a trend) the ATP production in fibroblast derived from a patient carrying a heterozygous OPA1 (R290Q/+) mutation which is homologous to the worm eat-3(R289Q) mutation. FIG. 9B) 10 μM Hemin (#17), 10 nM and 50 nM Tripterin (#17), and 5 μM and 50 μM Thiamine treatment significantly increases ATP production in fibroblast derived from a patient carrying a OPA1 (1403T) mutation.

FIGS. 10A-10B: Downregulating mitophagy does increase uaDf5 animals' single large-scale mitochondrial DNA (SLSMD) heteroplasmy level and increases mitochondrial stress. FIG. 10A) RNAi screen of mitophagy pathway genes on % mtDNA deletion (mtDNAA) heteroplasmy level. Mean (±SEM) of the % mtDNAA heteroplasmy level as assessed in 3 biological replicate experiments of untreated wildtype (hsp-6p::GFP), untreated SLSMD mutant tagged with a mitochondrial stress reporter (uaDf5;hsp-6p::GFP #1) treated with a control RNAi clone or RNAi against one of ten mitophagy pathway genes, namely atg-9, dct-1, pink-1, sqst-1, hrdl-1, mul-1, pdr-1, plastin-1, siah-1, and unc-51. All RNAi clones were obtained from a public RNAi clone library, except for plastin-1 that was newly generated here (two plastin clones were created, named plastin-1A and plastin-1B). FIG. 10B) RNAi screen of effects of knocking down mitophagy pathway genes on mitochondrial stress. Mean (±SEM) of the median fluorescence of the mitochondrial stress reporter hsp-6p::GFP was assessed in 3 biological replicate experiment of untreated wildtype (hsp-6p::GFP) worms, untreated uaDf5;hsp-6p::GFP #1 worms, and uaDf5;hsp-6p::GFP #1 worms treated with RNAi to knockdown various mitophagy pathway genes (atg-9, dct-1, pink-1, sqst-1, hrdl-1, mul-1, pdr-1, plastin-1, siah-1, unc-51). Mitochondrial stress was increased to the greatest degree in uadf5 animals with a heteroplasmic SLSMD when knocking down hrdl-1, siah-1, or unc-51 mitophagy pathway genes.

FIGS. 11A-11D: Mitochondrial stress screening assay assessing effects of therapeutic agents known to benefit mitochondrial respiratory chain complex I disease animal models in uaDf5 animals with a heteroplasmic SLSMD. FIG. 11A) 40 μM Carnitine significantly rescues mitochondrial stress in uaDf5 animals. Dichloroacetate (DCA) and higher doses of Folinic Acid show strong trends toward rescuing mitochondrial stress in uaDf5 animals. FIG. 11B) 25 mM Thiamine significantly rescues mitochondrial stress in uaDf5 animals. FIG. 11C) AICAR significantly rescues mitochondrial stress in uaDf5 animals. Lipoic acid reduces mitochondrial stress in uaDf5 animals to below that of wildtype controls. EtOH refers to ethanol; NAC refers to N-Acetylcysteine; DCA refers to dichloroacetate; and LiCL refers to lithium chloride. FIG. 11D) 4 replicate experiments show that 0.5 mM Hydralazine, 25 mM Thiamine, and 0.5 mM AICAR can rescue mitochondrial stress response in uaDf5 animals.

FIGS. 12A-12D: Screening mitophagy modulating compound library of 62 compounds from Medchemexpress assaying mitochondrial stress (hsp-6p::GFP) in uaDf5 animals. FIG. 12A) In the first experimental replicate of the screen, Vorinostat, GSK2578215A, Hemin and Tripterin rescued mitochondrial stress in uaDf5 animals at 25 μM. In the second replicate experiment of the screen, FIG. 12B) Hemin and FIG. 12C) Resveratrol rescued mitochondrial stress in the uaDf5 animals at 25 μM, FIG. 12D) Table summarizing the results of the duplicate screen, where a green tile indicates rescue and red tile indicates no effect.

FIGS. 13A-13D: validating the hits identified that passed the retest. Hemin and Tripterin reproducibly rescue mitochondrial stress in uaDf5 animals in a dose-dependent manner. FIG. 13A) Five compounds that passed the retest were assessed at 4 doses with wild type (blue), untreated/DMSO treated (uaDf5) strain #2 (salmon), Thiamine treated uaDf5 (purple), and drug treated uaDf5 (grey). Only Hemin and Tripterin reproducibly rescued mitochondrial stress response in a dose dependent manner. FIGS. 13B-13C) Six independent biological replicates show that in at least five different replicates both Hemin and Tripterin were able to rescue mitochondrial stress response in uaDf5 animals. FIG. 13D) Combination therapies were tested using Thiamin, Hemin and Tripterin. Thiamine and Tripterin in combination can rescue the mitochondrial stress response phenotype far more effectively than either alone. Hemin and Tripterin also shows greater efficacy in combination.

FIG. 14: Summary table of the significant results on uaDf5 animal mitochondrial stress induction shown in FIGS. 11-13.

FIG. 15: uaDf5 mtDNAA heteroplasmy upon 24-hour treatment. The uaDf5 mtDNAA heteroplasmy was tested in 8-12 biological replicates after 24-hour in untreated (blue), 50 mM thiamine (green), 0.13% DMSO (light orange) as a control for 25 μM Hemin (orange) and Tripterin (dark orange) treatment, 0.26% DMSO (light pink) as a control for 50 μM Hemin (pink) and Tripterin (dark pink) treatment. The median and quartile range is graphed.

FIGS. 16A-16D: Developing primers for testing heteroplasmy in patient samples. FIG. 16A) Three sets of primers were designed. One set to capture the entire mtDNA genome, one set to capture only wild-type DNA as it amplifies a portion absent in SLSMD per cell line, and the third set was designed for each deletion to amplify across the deletion to assess the mutant molecules. FIG. 16B) Table of mtDNA genes in SLSMD deleted region. FIG. 16C) Two primer sets that capture only functioning wild-type DNA. FIG. 16D) Only the set of primers for cell line 1 amplified across the deleted region (Del4) in the SLSMD molecule. The primers for the other fibroblast lines did not amplify.

FIG. 17: Growth of healthy versus mutated patient cells normalized to DMEM+Uridine.

FIG. 18: A reporter to quantify mtDNA deletion heteroplasmy levels in living C. elegans uaDf5 animals harboring a SLSMD. The reporter is composed of two components, each with motifs for (i) mitochondrial localization (MTS), (ii) DNA sequence recognition (TALE: Transcription activator-like effector), and (iii) visualization (split fluorophore).

FIG. 19A-19C: FIG. 19A) The C. elegans mitochondrial genome is a 12.8 kb (black line circle) circular piece of DNA, which encodes 12 proteins (arrows), 2 rRNAs (arrows), and 23 tRNAs (bars). The uaDf5 animal are heteroplasmic for both the full length wild-type mtDNA and the mutant mtDNA, which has a 3.1 kb deletion as indicated by the black segment). FIG. 19B) The WT mtDNA genome will bind both the WT (blue rectangle) and mutant (orange rectangle) TALE designs. Only the pair designed to bind DNA sequence absent in the mutant DNA (black) will be in proximity and allow for the split protein (red) to reconstitute and fluoresce (red halo, indicative of wild-type mtDNA genomes). FIG. 19C) The mutant mtDNA genome should only bind the TALE pairs (orange rectangle) that recognize mtDNA sequence flanking the deletion (blue and green bars) at which point, the split fluorescent protein (green) will reconstitute and fluoresce (green halo, indicative of deleted mDNA genomes).

FIG. 20: The TALE module are repeats of 34 amino acid sequence where the identity of the 12^{th} and 13^{th} amino acids, repeat variable di-residue (RVD) confer DNA base recognition. The inset table lists the known RVD amino acids that recognize distinct nucleic acids.

FIG. 21A-21B: FIG. 21A) The available paired TALE constructs (teal and purple), where the right TALE monomer is designed to recognize mutant human mtDNA with a single base pair mutation (dark orange block), and both TALE monomers are fused to Fokl (Blue circles) designed to make a double-stranded cut in the mtDNA. FIG. 21B) The TALE paired constructs where the right TALE monomer is reengineered to recognize WT human mtDNA and both TALE monomers are fused to the split fluorescent protein (red) (Prior Art).

### DETAILED DESCRIPTION OF THE INVENTION

Mitophagy, a mitochondrial quality control mechanism enabling the degradation of damaged and superfluous mitochondria, prevents such detrimental effects and reinstates cellular homeostasis in response to stress. To date, there is increasing evidence that mitophagy is significantly impaired in several human pathologies including mitochondrial disease, aging and age-related diseases such as neurodegenerative disorders, cardiovascular pathologies and cancer. Upregulating mitophagy, in the context of certain diseases caused by mutations in the mitochondrial genome, for example Single Large-Scale mtDNA Deletion Syndrome (SLSMDS), could reduce the frequency of mtDNA with mutations and ameliorate disease symptoms.

Downregulating mitophagy, in the context of other diseases caused by decreased mitochondrial genome stability or in diseases where mitophagy is upregulated, e.g., OPA1, could stabilize effects of mtDNA mutation(s) known to cause disease in humans: (e.g., R289Q or V328I mutant alleles of OPA1).

We have established novel ways to screen C. elegans models of mitochondrial disease for gene targets and pharmacologic compounds to therapeutically treat these complex disorders. Studies in a novel C. elegans (humanized disease gene) model of OPA1 (autosomal dominant optic atrophy (ADOA), as well as ADOA plus that has multi-system involvement) disease and a newly validated disease model of heteroplasmic single-large scale mtDNA deletion syndromes (SLSMDS, such as is the cause of Pearson Syndrome (PS), Kearns Sayre Syndrome (KSS), and Chronic Progressive External Ophthalmoplegia (CPEO) and CPEO plus that has multi-system involvement) are described. Gene knockdown studies by feeding RNA interference (RNAi) of diverse mitophagy pathway genes was employed, along with pharmacologic studies of mitophagy pathway modulators, in stable genetic mutant C. elegans (invertebrate nematode animal) models of major mitochondrial disease classes. These studies include the specific modulation of mitophagy pathway genes whose knockdown worsens mutation heteroplasmy levels and animal activity in mtDNA heteroplasmic SLSMD models, while rescuing animal activity and/or mitochondrial stress in nuclear-encoded OPA1 disease. In addition, modulation of these pathways with specific therapies highlights novel candidate therapies for these primary mitochondrial diseases as well as respiratory chain (e.g., complex I such as the NDUFS2 and other subunit or assembly gene; complex II, complex III, complex IV, complex V, multiple respiratory chain complex disorders) diseases. This work holds broad applications to monitoring in vivo mitophagy activity in C. elegans and modulating mitophagy in a rheostat-based fashion to achieve therapeutic results at variable levels in different forms and contextual states (e.g., stressed or baseline conditions) of mitochondrial disease.

### Definitions

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, C. elegans, zebrafish, mice, rats, hamsters, and primates.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

A “genetic or protein alteration” as used herein, includes without limitation, naturally occurring mutations, chemically induced mutations, genetic alterations generated via introduction of siRNA, antisense oligonucleotides, Talens, and CRISPR-CAS 9 targeted gene constructs. Protein alterations can be generated via pharmacological inhibition or modification of proteins involved in mitochondrial respiratory chain function.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented.

As used herein, “amcliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routinc statistical tests.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid-based molecule which exhibits the capacity to modulate the activity of a mitochondrial disease associated gene.

The term “synergy” or “synergistic” refers to the interaction or cooperation of two or more substances, or other agents to produce a combined effect greater than the sum of their separate effects. In certain embodiments, the combinations provided herein act synergistically.

As used herein, “mitochondrial related disorders” related to disorders which are due to abnormal mitochondria such as for example, a mitochondrial genetic mutation, enzyme pathways etc. Examples of disorders include and are not limited to: loss of motor control, muscle weakness and pain, imbalance, coordination problems, peripheral neuropathy, migraines, headaches, cognitive problems, memory problems, strokes, seizures, autonomic dysfunction, sleep problems, exercise intolerance, chronic fatigue, gastro-intestinal disorders and swallowing difficulties, poor growth, cardiac disease, liver disease, diabetes, respiratory complications, visual/hearing problems, lactic acidosis, developmental delays and susceptibility to infection. The mitochondrial abnormalities give rise to “mitochondrial diseases” which include, but not limited to: ADOA: Autosomal Dominant Optic Atrophy; AD: Alzheimer's Disease; ADPD: Alzheimer's Disease and Parkinsons's Disease; AMDF: Ataxia, Myoclonus and Deafness CIPO: Chronic Intestinal Pseudo-obstruction with myopathy and Opthalmoplegia; CPEO: Chronic Progressive External Opthalmoplegia; DEAF: Maternally inherited deafness or aminoglycoside-induced Deafness; DEMCHO: Dementia and Chorea; DMDF: Diabetes Mellitus & Deafness; Exercise Intolerance; ESOC: Epilepsy, Strokes, Optic atrophy, & Cognitive decline; FBSN: Familial Bilateral Striatal Necrosis; FICP: Fatal Infantile Cardiomyopathy Plus, a MELAS-associated cardiomyopathy; GER: Gastrointestinal Reflux; KSS: Kearns Sayre Syndrome LDYT: Leber's hereditary optic neuropathy and Dystonia; LHON: Leber Hereditary Optic Neuropathy; LIMM: Lethal Infantile Mitochondrial Myopathy; MDM: Myopathy and Diabetes Mellitus; MELAS: Mitochondrial Encephalomyopathy, Lactic Acidosis, and Stroke-like episodes; MEPR: Myoclonic Epilepsy and Psychomotor Regression; MERME: MERRF/MELAS overlap discasc; MERRF: Myoclonic Epilepsy and Ragged Red Muscle Fibers; MHCM: Maternally Inherited Hypertrophic CardioMyopathy; MICM: Maternally Inherited Cardiomyopathy; MILS: Maternally Inherited Leigh Syndrome; Mitochondrial Encephalocardiomyopathy; Mitochondrial Encephalomyopathy; MM: Mitochondrial Myopathy; MMC: Maternal Myopathy and Cardiomyopathy; Multisystem Mitochondrial Disorder (myopathy, encephalopathy, blindness, hearing loss, peripheral neuropathy); NARP: Neurogenic muscle weakness, Ataxia, and Retinitis Pigmentosa; alternate phenotype at this locus is reported as Leigh Disease; NIDDM: Non-Insulin Dependent Diabetes Mellitus; PEM: Progressive Encephalopathy; PME: Progressive Myoclonus Epilepsy; PS: Pearson syndrome; RTT: Rett Syndrome; SIDS: Sudden Infant Death Syndrome. The “OPA1 mitochondrial dynamin like GTPase (OPA1)” is a nuclear-encoded mitochondrial protein with similarity to dynamin-related GTPases. The encoded protein localizes to the inner mitochondrial membrane and helps regulate mitochondrial stability and energy output. This protein also sequesters cytochrome c. Mutations in this gene have been associated with optic atrophy type 1, which is a dominantly inherited optic neuropathy (ADOA) resulting in progressive loss of visual acuity, leading in many cases to legal blindness, and in some cases causes additional multi-system problems including but not limited to sensorineural hearing loss, deafness, myopathy, and neuropathy. Inhibition of mitophagy is efficacious for amelioration of symptoms for this mitochondrial disease.

“Mitochondrial DNA (mtDNA) deletion syndromes (e.g., Single Large-Scale mtDNA Deletion Syndrome (SLSMDS)” predominantly comprise three overlapping phenotypes that are usually simplex (i.e., a single occurrence in a family), but rarely may be observed in different members of the same family or may evolve from one clinical syndrome to another in a given individual over time. The three classic phenotypes caused by mtDNA deletions are Kearns-Sayre syndrome (KSS), Pearson syndrome (PS), and chronic progressive external ophthalmoplegia (CPEO). Activation of mitophagy is efficacious for amelioration of symptoms for this mitochondrial disease.

KSS is a progressive multisystem disorder defined by onset before age 20 years, pigmentary retinopathy, and CPEO; additional features include cerebellar ataxia, impaired intellect (intellectual disability, dementia, or both), sensorineural hearing loss, ptosis, oropharyngeal and esophageal dysfunction, exercise intolerance, muscle weakness, cardiac conduction block, and endocrinopathy.

Pearson syndrome (PS) is characterized by sideroblastic anemia and exocrine pancreas dysfunction, often with lactic acidosis, and may be fatal in infancy without appropriate hematologic management.

PEO is characterized by ptosis, impaired eye movements due to progressive paralysis of the extraocular muscles (ophthalmoplegia), oropharyngeal weakness, and variably severe proximal limb weakness with exercise intolerance.

Rarely, a mtDNA deletion, especially when at high heteroplasmy levels, can manifest as Leigh syndrome.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

The terms “polynucleotide”, “nucleotide”, “nucleotide sequence”, “nucleic acid”, and “oligonucleotide” are used interchangeably in this disclosure. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: single-, double-, or multi-stranded DNA or RNA, genomic DNA, cDNA, DNA-RNA hybrids, or a polymer comprising purine and pyrimidine bases or other natural, chemically, or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

Nucleic acid molecules that inhibit expression of a gene or nucleic acid can be referred to as “inhibitory nucleic acid” (referring to their composition). Inhibitory nucleic acid technologies are known in the art and include, but are not limited to, antisense oligonucleotides, catalytic nucleic acids such as ribozymes and deoxyribozymes, aptamers, triplex forming nucleic acids, external guide sequences, and RNA interference molecules (RNAi), particularly small nucleic acid molecules, such as short interfering nucleic acid (siNA), short interfering RNA (siRNA), double-stranded RNA (dsRNA), micro-RNA (mRNA), and short hairpin RNA (shRNA) molecules capable of mediating RNA interference (RNAi).

An inhibitory nucleic acid can reduce expression of a protein encoded by a gene selected from atg-9, dct-1, pink-1, sqst-1, hrdl-1, mul-1, pdr-1, plastin-1, siah-1, unc-51, herein after referred to as mitophagy modulator proteins. The inhibitory nucleic acid can reduce expression of an mRNA sequence encoding the mitophagy modulator proteins or genomic DNA encoding the mRNA.

The expression or amount of a mitophagy modulator protein can be reduced in some cases using RNA interference, whereby double-stranded RNA (dsRNA, also referred to herein as siRNAs or ds siRNAs, for double-stranded small interfering RNAs) induces the sequence-specific degradation of targeted mRNA in cells (Hutvagner and Zamore, Curr. Opin. Genet. Dev.: 12, 225-232 (2002); Sharp, Genes Dev., 15:485-490 (2001)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), or by micro-RNAs (miRNA), functional small-hairpin RNA (shRNA), or other dsRNAs which can be expressed in vivo using DNA templates with RNA polymerase III promoters (Zeng et al., Mol. Cell 9:1327-1333 (2002); Paddison et al., Genes Dev. 16:948-958 (2002); Lee et al., Nature Biotechnol. 20:500-505 (2002); Paul et al., Nature Biotechnol. 20:505-508 (2002); Tuschl, T., Nature Biotechnol. 20:440-448 (2002); Yu et al., Proc. Natl. Acad. Sci. USA 99 (9): 6047-6052 (2002); McManus et al., RNA 8:842-850 (2002); Sui et al., Proc. Natl. Acad. Sci. USA 99 (6): 5515-5520 (2002)).

In a preferred embodiment, the inhibitory nucleic acid is an siRNA. In one embodiment, the inhibitory nucleic acid has 100% sequence identity with at least a part of the target mRNA. However, inhibitory nucleic acids having 70%, 80% or greater than 90% or 95% sequence identity may be used. Thus, sequence variations that might be expected due to genetic mutation, strain polymorphism, or evolutionary divergence can be tolerated. siRNA specific for the mitophagy modulator proteins are commercially available from Dharmacon, upon request, along with other companies that will generate interfering RNAs for a specific gene. Thermo Electron Corporation (Waltham, MA) has launched a custom synthesis service for synthetic short interfering RNA (siRNA). Each strand is composed of 18-20 RNA bases and two DNA bases overhang on the 3′ terminus. As mentioned, Dharmacon, Inc. (Lafayette, CO) provides siRNA duplexes using the 2′-ACE RNA synthesis technology. Qiagen (Valencia, CA) uses TOM-chemistry to offer siRNA with high individual coupling yields (Li, et al., Nat. Med., 11(9): 944-951 (2005).

In some forms the inhibitor of the mitophagy protein modulator is an antisense oligonucleotide. An “antisense” nucleic acid sequence (antisense oligonucleotide) can include a nucleotide sequence that is complementary to a “sense” nucleic acid encoding a protein, e.g., complementary to the coding strand of a double-stranded cDNA molecule or complementary to a target sequence encoding the mitochondrial modulator protein. Antisense nucleic acid sequences and delivery methods are well known in the art (Goodchild, Curr. Opin. Mol. Ther., 6(2): 120-128 (2004); Clawson, et al., Gene Ther., 11(17): 1331-1341 (2004)), which are incorporated herein by reference in their entirety. An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In some forms the inhibitor of mitophagy modulator protein expression is a ribozyme specific for a nucleic acid encoding the protein. Ribozymes are a type of RNA that can be engineered to enzymatically cleave and inactivate other RNA targets in a specific, sequence-dependent fashion. Ribozymes and methods for their delivery are well known in the art (Hendry, et al., BMC Chem. Biol., 4(1): 1 (2004); Grassi, et al., Curr. Pharm. Biotechnol., 5(4): 369-386 (2004); Bagheri, et al., Curr. Mol. Med., 4(5): 489-506 (2004); Kashani-Sabet M., Expert Opin. Biol. Ther., 4(11): 1749-1755 (2004), each of which are incorporated herein by reference in its entirety. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art.

“Native RNA” is naturally occurring RNA (i.e., RNA with normal C, G, U and A bases, ribose sugar and phosphodiester linkages).

As used herein, “antisense activity” means any detectable and/or measurable change attributable to the hybridization of an antisense compound to its target nucleic acid.

As used herein, “detecting” or “measuring” means that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

As used herein, “detectable and/or measurable activity” means a measurable activity that is not zero.

As used herein, “essentially unchanged” means little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

As used herein, “expression” means the process by which a gene ultimately results in a protein. Expression includes, but is not limited to, transcription, post-transcriptional modification (e.g., splicing, polyadenylation, addition of 5′-cap), translation, and post-translational modification.

As used herein, “translation” means the process in which a polypeptide (e.g. a protein) is translated from an mRNA. In certain embodiments, an increase in translation means an increase in the number of polypeptide (e.g. a protein) molecules that are made per copy of mRNA that encodes said polypeptide.

As used herein, “targeting” or “targeted to” means the association of an inhibitory nucleic acid compound to a particular target nucleic acid molecule or a particular region of a target nucleic acid molecule. For example, an antisense compound targets a target nucleic acid if it is sufficiently complementary to the target nucleic acid to allow hybridization under physiological conditions.

As used herein, “mismatch” means a nucleobase of a first oligomeric compound that is not capable of pairing with a nucleobase at a corresponding position of a second oligomeric compound, when the first and second oligomeric compound are aligned. Either or both of the first and second oligomeric compounds may be oligonucleotides.

The terms “construct”, “cassette”, “expression cassette”, “plasmid”, “vector”, or “expression vector” is understood to mean a recombinant nucleic acid, generally recombinant DNA, which has been generated for the purpose of the expression or propagation of a nucleotide sequence(s) of interest or is to be used in the construction of other recombinant nucleotide sequences.

The term “promoter” or “promoter polynucleotide” is understood to mean a regulatory sequence/element or control sequence/element that is capable of binding/recruiting an RNA polymerase and initiating transcription of sequence downstream or in a 3′ direction from the promoter. A promoter can be, for example, constitutively active, or always on, or inducible in which the promoter is active or inactive in the presence of an external stimulus. Example of promoters include T7 promoters or U6 promoters.

The term “operably linked” can mean the positioning of components in a relationship which permits them to function in their intended manner. For example, a promoter can be linked to a polynucleotide sequence to induce transcription of the polynucleotide sequence.

The terms “complementarity” or “complement” refer to the ability of a nucleic acid to form hydrogen bond(s) with another nucleic acid sequence by cither traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 4, 5, and 6 out of 6 being 66.67%, 83.33%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 40%, 50%, 60%, 62.5%, 70%, 75%, 80%, 85%, 90%, 95%, 97%, 98%, 99%, or 100%, or percentages in between over a region of 4, 5, 6, 7, and 8 nucleotides, or refers to two nucleic acids that hybridize under stringent conditions.

A nucleic acid as described herein can be “modified” to increase stability in vivo. Such modifications include, without limitation, sugar modifications such as 2′fluoro, 2′-O-methyl, 2′-NH₂. The phosphodiester backbone linkage can also be substituted with phosphorothioate as disclosed herein, but other backbone modifications such as triazole linked, or phNA are known to the skilled artisan. Additionally, modified bases can be employed, including without limitation, 7-deaza-dA, and carboxamide-dU. These 2′ substituents influence ASO molecular conformation, resulting in improved RNA target binding affinity and, with the exception of 2′-fluoro, increased nuclease resistance. Like phosphorodiamidate-modified morpholino oligomers, fully 2′-modified ASOs do not recruit RNase H1. Although less common, nucleobase modifications can also be incorporated into ASO design. Numerous modifications have been identified, e.g., replacing cytosine with 5-methylcytosine has proved beneficial: 5-methylcytosine substitution reduces ASO immunostimulatory effects without compromising Watson-Crick complementarity. Third-generation modifications more extensively alter ASO chemistry to further enhance stability and potency post-administration and provide greater control over both target affinity and cellular tropism. These modifications may alter nucleoside connectivity and restrict ASO stereochemistry, as in locked nucleic acids, constrained ethyl nucleoside analogues and artificial amido-bridged nucleic acids; change the backbone charge (phosphorodiamidate-modified morpholino oligomers are third-generation modified ASOs); or link ASOs to ligands, as in cholesterol- and GalNAc-conjugated ASOs.

In some aspects, the invention provides methods comprising delivering one or more polynucleotides, such as or one or more vectors as described herein (e.g., encoding all or portions of the base editing complexes discussed below), one or more transcripts thereof, and/or one or proteins transcribed therefrom, to a host cell. In some aspects, the invention further provides cells produced by such methods, and organisms (such as animals, plants, or fungi) comprising or produced from such cells. Conventional viral and non-viral based gene transfer methods can be used to introduce nucleic acids in mammalian cells or target tissues. Such methods can be used to administer nucleic acids encoding components of a base editing system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51 (1): 31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration).

The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and ψ2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line.

In one aspect, the invention provides for methods of modifying a target polynucleotide in a eukaryotic cell, which may be in vivo, ex vivo or in vitro. In some embodiments, the method comprises sampling a cell or population of cells from a human or non-human animal and modifying the cell or cells. Culturing may occur at any stage ex vivo. The cell or cells may be re-introduced into the human or non-human animal.

### Pharmaceutical Formulations

For clinical applications, pharmaceutical compositions will be prepared in a form appropriate for the intended application. Generally, this will entail preparing compositions (e.g., expression vector) that are essentially free of pyrogens, as well as other impurities that could be harmful to humans or animals.

One will generally desire to employ appropriate salts and buffers to render drugs stable and allow for uptake by target cells. Aqueous compositions of the present disclosure comprise an effective amount of the drug dissolved or dispersed in a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the vectors or cells of the compositions.

The active compositions of the present disclosure may include classic pharmaceutical preparations. Administration of these compositions according to the present disclosure may be via any common route so long as the target tissue is available via that route. This includes oral, nasal, or buccal, as well as through nasal feeding tubes or gastrostomy or jejunual ports and tubes that are commonly needed in primary mitochondrial disease patients. Alternatively, administration may be by intradermal, subcutaneous, intramuscular, intraocular, intraperitoneal or intravenous injection. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The compositions of the present disclosure generally may be formulated in a neutral or salt form. Pharmaceutically acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics Standards.

### Therapies

In another embodiment, combinatorial treatment of mitochondrial disease is contemplated. Combinations may be achieved by treating patients with a single composition or pharmacological formulation that includes two or more agents, or by treating the patient with distinct compositions or formulations, at the same time, wherein each composition includes a distinct agent. Alternatively, the various agents may be given in a staggered fashion ranging from minutes, to hours, to weeks. In such embodiments, one would generally ensure that the period of time between each delivery was such that the agents would still be able to exert an advantageously combined effect on the cell or subject. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Furthermore, multiple administrations of the cocktail itself are contemplated, such as in an ongoing or chronic basis. The administrations may be twice daily, daily, twice weekly, weekly, every other week, or monthly. They may also be administered for therapeutic purposes to mitochondrial disease patients who are acutely decompensating on a continual or more frequent basis in an acute medical setting (emergency department, intensive care unit, etc).

In another aspect, the present disclosure provides compositions comprising one or more of compounds as described above and an appropriate carrier, excipient or diluent. The exact nature of the carrier, excipient or diluent will depend upon the desired use for the composition and may range from being suitable or acceptable for veterinary uses to being suitable or acceptable for human use. The composition may optionally include one or more additional compounds.

When used to treat or prevent such diseases, the compounds described herein may be administered singly, as mixtures of one or more compounds or in mixture or combination with other agents useful for treating such diseases and/or the symptoms associated with such diseases.

The compounds may also be administered in mixture or in combination with agents useful to treat other disorders or maladies, such as steroids, membrane stabilizers, leukotriene synthesis and receptor inhibitors, inhibitors of IgE isotype switching or IgE synthesis, IgG isotype switching or IgG synthesis, β-agonists, tryptase inhibitors, aspirin, COX inhibitors, methotrexate, anti-TNF drugs, retuxin, PD4 inhibitors, p38 inhibitors, PDE4 inhibitors, and antihistamines, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically. The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, oral, topical, ocular, buccal, systemic, nasal, injection, transdermal, rectal, vaginal, etc., or a form suitable for administration by inhalation or insufflation.

For oral administration, the pharmaceutical compositions may take the form of, for example, lozenges, tablets or capsules prepared by conventional means with pharmaceutically acceptable excipients such as binding agents (e.g., pre-gelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™. or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For buccal administration, the compositions may take the form of tablets or lozenges formulated in conventional manner.

For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethylsulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s).

The following materials and methods are provided to facilitate practice of the invention.

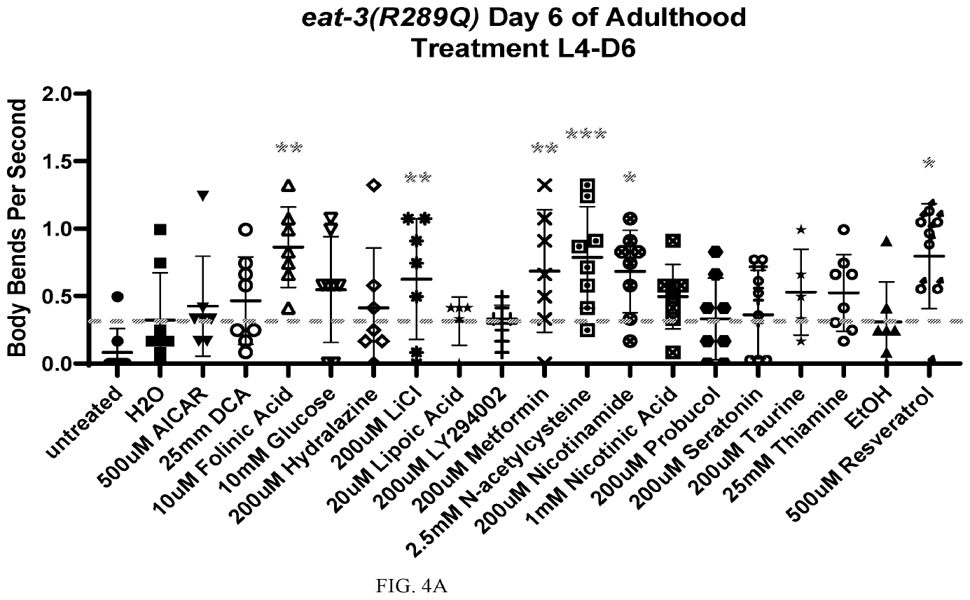

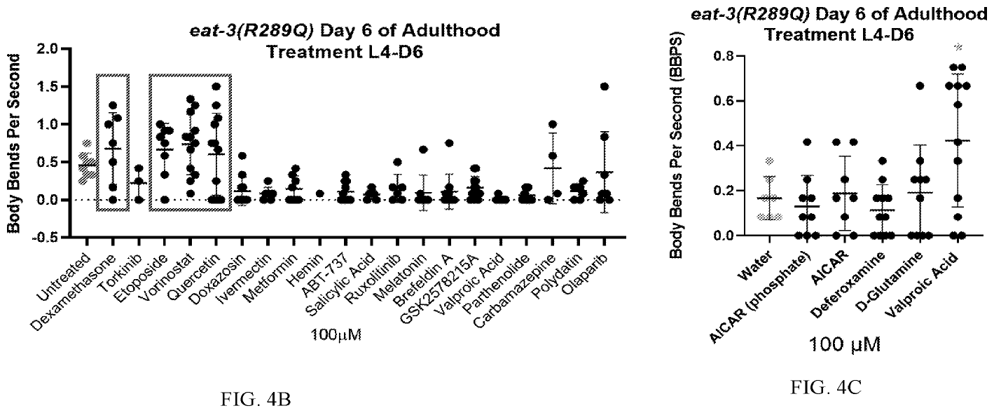

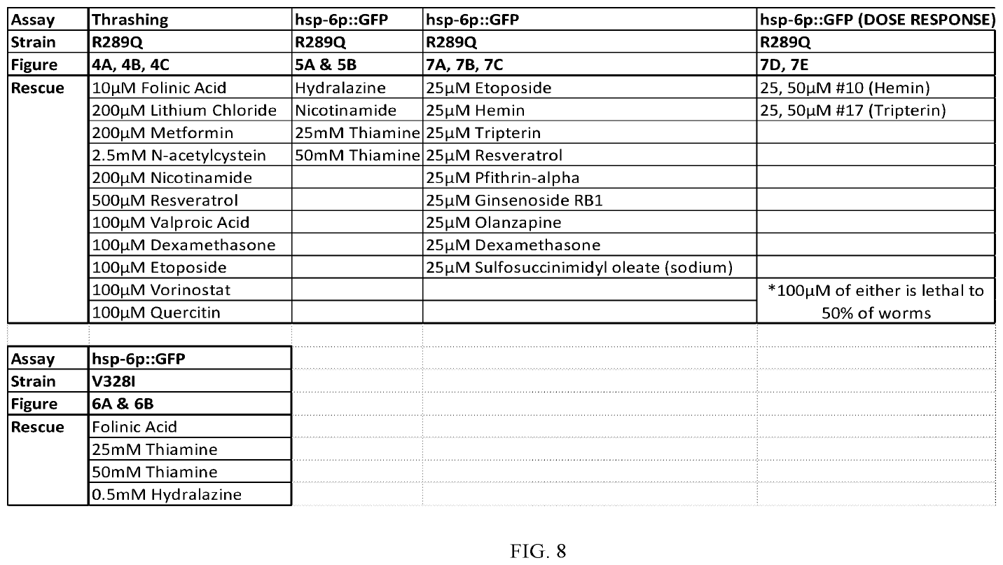

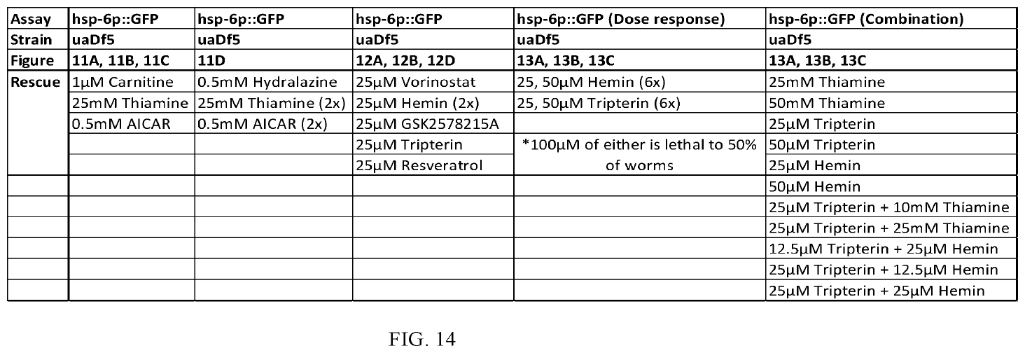

For C. elegans animal studies, worms will be grown on Nematode Growth Media (NGM) agar plates. For new drugs not previously studied in our research laboratory, the maximal possible exposure as well as two log-order lower concentrations will be tested to determine the maximal concentration of each candidate drug and nutrient treatment being tested that does not delay wild-type worm development from the L1 larval stage through first-day of young adulthood (as defined by eggs being laid on the plate). The highest non-toxic concentration in this assay will then be used to test drug effects on lifespan. The same drugs and similar molar concentration range will be tested in C. elegans as are detailed in the tables in FIGS. 8 and 14 and described in FIGS. 4-7 and 11-13.

In certain approaches, animal lifespan can be evaluated in the C. elegans opa-1 mutant models. The lifespan of each opa-mutant strain studied will be determined (typically 2 weeks on average and 4 weeks maximal in healthy wild-type (N2 Bristol) worms). Lifespan analyses will subsequently be performed on our automated robotic system (WormCamp) to evaluate effects of candidate drugs. All lifespan data can be statistically analyzed, with subsequent confirmation of drugs having significant effects in additional analyses, both individually and in combination with other drugs showing therapeutic benefit.

Mitochondrial physiology can also be evaluated in the C. elegans opa-1 mutant worms, using a combination of worms with fluorescently tagged mitochondria genetic constructs and mitochondrial-targeted dyes. Mitochondrial mass and membrane potential will be relatively quantified in a high-throughput fashion by whole worm fluorescence intensity analysis in levamisole-paralyzed nematodes using automated Biosorter (COPAS) analyses at 20° C. Total oxidative stress is similarly evaluated using the dihydroethidium (DHE) fluorescent dye. Should any of these parameters prove to be abnormal in the opa-1 mutant worms, then that parameter(s) will be used as a secondary outcome measure to evaluate drug and treatment effects in opa-1 disease.

Depending on the results of candidate therapy screen, high throughput screening will be performed in our laboratory using our automated CX5 (ThermoFisher) high-content imager and/or Biosorter animal sorting system to identify in an available library of FDA-approved drugs and/or natural compounds that improve cell survival and/or mitochondrial function in OPA1 disease worms. Cellular ATP levels, electron transport chain (ETC) enzyme activities, and other biochemical analyses of drug effects (such as NADH, NAD+, GSSG: GSH glutathione levels and activity states, metabolite profiling, etc) can be performed in our laboratory to elucidate disease mechanisms and treatment effects.

Feeding RNAi knockdown screen of mitophagy pathway genes was done by exposing uaDf5 embryos with heteroplasmic SLSMDs to RNAi modulating bacteria for 4 days prior to assessment.

SLSMD heteroplasmy levels were assessed using quantitative PCR (qPCR) with SYBR Green reagents to measure nuclear DNA, total mtDNA levels, and mutant mtDNA levels in populations of 30 C. elegans synchronized animals.

Neuromuscular function/Thrashing assay was assessed by counting body bends of animals thrashing in liquid media at 7 days of adulthood. The higher number of thrashes, or body bends, per second, the healthier the neuromuscular function of a given animal.

Fecundity was assessed by counting the total progeny produced by a single worm over the first 4 days of adulthood.

Mitochondrial stress was assessed using a genetic reporter strain with GFP expression driven by the hsp-6 promoter, which is activated by the mitochondrial unfolded protein response (UPR^{mt}), a form of mitochondrial stress. The fluorescence was measured on the BioSorter® Large Particle Flow Cytometer from Union Biometrica using FlowPilot™ software.

High-throughput drug screening was performed by assessing mitochondrial stress with and without drug treatment using the CX5, a high-content screening (HCS) imaging platform from ThermoFisher. All screens shown in this application were performed in C. elegans wild-type or genetic mutant worm strains using thrashing and/or mitochondrial stress. Each hit will first be re-confirmed using the assay it was identified. The confirmed hits will then be then tested using mtDNA content, respiration, and thrashing in eat-3 mutant animals having the eat-3 (R289Q) mutation, which is the worm homolog of OPA1 with a human-disease causing allele. mtDNA analysis will be performed in uaDf5 animals. uaDf5 is a repurposed previously described strain that provides a suitable C. elegans model to study heteroplasmic Single Large-Scale mtDNA Deletion Syndromes (SLSMDS). This strain carries both wild-type and mutant mitochondrial genome (mtDNA). The mutant mtDNA carry a 3.1 kb deletion. Note currently there are no SLSMD models in zebrafish. The confirmed therapeutic gene target and/or therapeutic compound hits will be tested in the fish using an OPA1 vision defect. Hits in the OPA1 fish will be tested in human patient OPA1 fibroblast cell lines. Hits in the uaDf5 worms will be directly tested in the SLSMD human patient fibroblast cell lines.

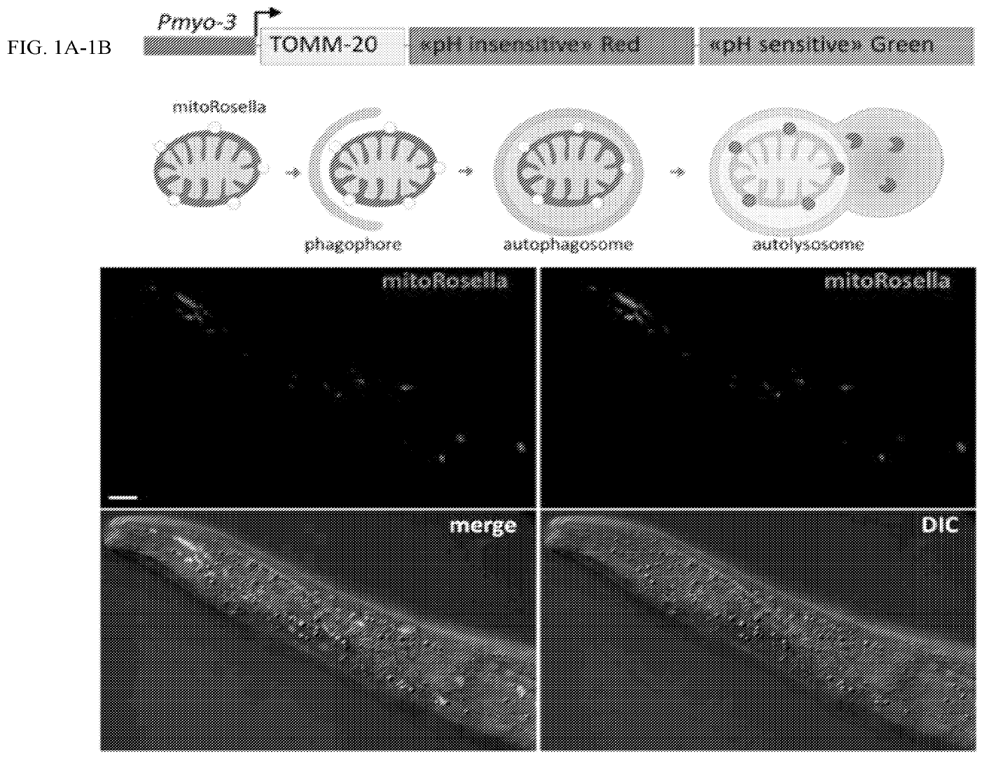

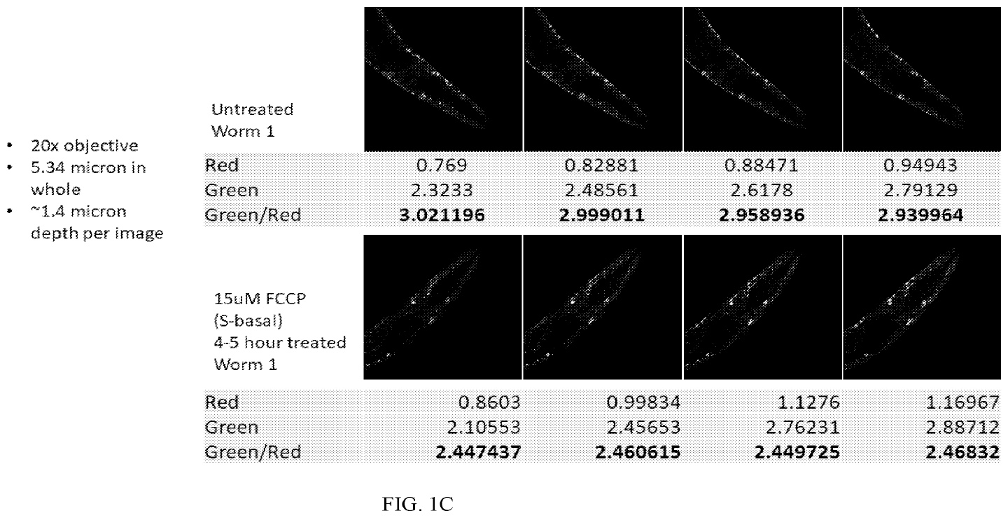

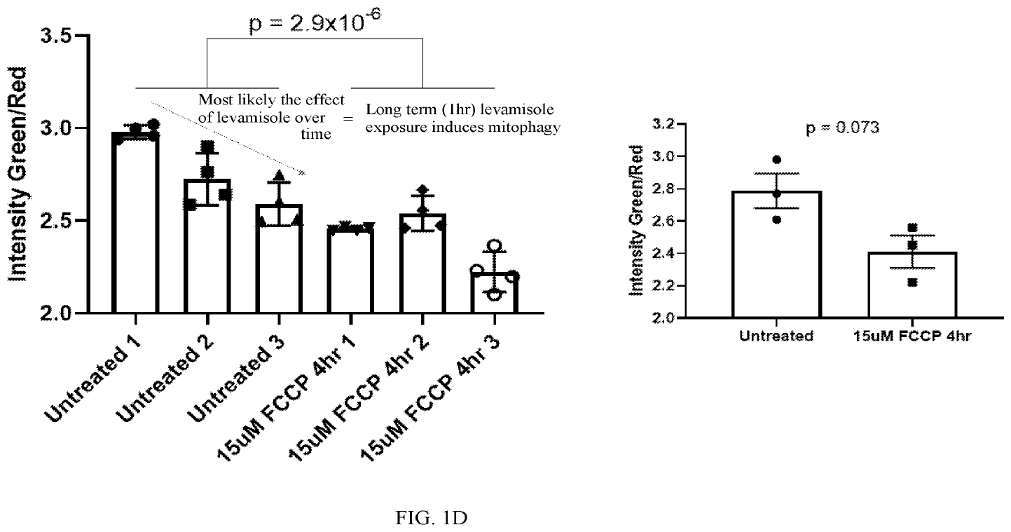

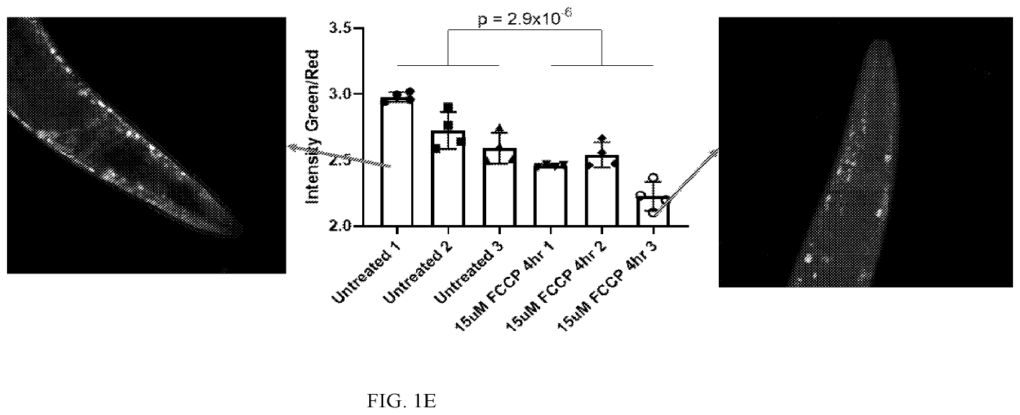

Mitophagy can be visualized using many different approaches. See for example Charmpilas et al., Methods of Molecular Biology (2018) 1759:151-160 who describe visualization of mitophagy in C. elegans using IR1631, a genetic fluorescent model. See FIGS. 1A-1E, which illustrate how the IR631 system can be used to advantage in the screening assays described herein.

Sun et al. describe “A fluorescence-based imaging method to measure in vitro and in vivo mitophagy using mt-Keima” in Nat Protoc (2017) 12:1576-1587 for use in the mouse. mtKeima will be adapted for use in a C. elegans model. We will also employ a mtKeima model for use in Fish. See Wrighton et al. J Cell Sci (2021) 134(4): jcs256255. Another approach entails use of Dojindo Mitophagy Dye, which is commercially available by Dojindo.com on the world wide web at dojindo.com/product/mitophagy-detection-kit/. The dye has previously been used in cells and will be repurposed here for use in worms and fish.

The following examples are provided to illustrate certain embodiments of the invention. They are not intended to limit the invention in any way.

### Example I

Development of Efficacious Agents for the Treatment of Mitochondrial Disorders Associated with Aberrantly Increased Mitophagy Induction and/or Reduced mtDNA Genome Stability

At least 240 pathogenic variants (aka ‘mutations’) in the OPA1 gene have been found to cause optic atrophy type 1. This condition typically results in vision loss beginning in childhood that worsens over time. Affected individuals may also have problems with color vision, particularly distinguishing between shades of blue and green. Most OPA1 gene mutations associated with optic atrophy type 1 are related to the presence of a premature stop codon in the nucleic acid (e.g., 2826delT) encoding the OPA1 protein. As a result, an abnormally small protein is produced, which is unstable and degraded quickly. OPA1 gene mutations are associated with aberrant mitochondrial structure and function. The mitochondria become misshapen and disorganized and have reduced energy-producing capabilities. The maintenance of mtDNA may also be impaired, resulting in mtDNA mutations that also contribute to mitochondrial dysfunction. Cells that contain these poorly functioning mitochondria are more susceptible to apoptosis. In particular, cells within the retina called retinal ganglion cells die over time. Specialized extensions of retinal ganglion cells, called axons, form the optic nerves, so when retinal ganglion cells die, the optic nerves break down (atrophy) and cannot transmit visual information to the brain. As the optic nerves atrophy, vision worsens, leading to the signs and symptoms of optic atrophy type 1.

While the OPA1 protein is found in cells throughout the body, retinal ganglion cells appear to be particularly sensitive to the effects of OPA1 gene mutations. These cells have especially high energy requirements that make them more likely to malfunction and die when there are changes in mitochondrial function and decreases in energy production. Autosomal dominant optic atrophy (ADOA), a form of progressive bilateral blindness due to loss of retinal ganglion cells and optic nerve deterioration, arises predominantly from mutations in the nuclear gene for the mitochondrial GTPase, OPA1. OPA1 localizes to mitochondrial cristae in the inner membrane where electron transport chain (ETC) complexes are enriched. While OPA1 has been characterized for its role in mitochondrial cristae structure and organelle fusion, possible effects of OPA1 on mitochondrial function have not been determined.

In the present example, the evaluation of mitochondrial targeted therapies particularly for the treatment of mitochondrial OPA1 mitochondrial disease is described. First, we describe efforts to establish and characterize informative translational disease models. Second, these models are then employed to objectively and efficiently screen the efficacy and toxicity of individual therapeutic targets and agents that have been empirically used in mitochondrial disease or shown promise in our prior mitochondrial disease models. Third, optimal therapeutic combinations are then identified and assessed in personalized clinical trials in an affected patient either prior to or upon symptom onset. The specific disease models described in this example can include human fibroblasts from an OPA1 disease patient(s) and healthy controls, along with C. elegans (worms, invertebrate animal) and zebrafish (D. rerio, vertebrate animal) models of OPA1 disease. Using this system we can establish a paradigm for precision therapeutic development for individual mitochondrial disease patients with distinct genetic etiologies, where therapies will first be tested and prioritized in human patient cell and animal models (C. elegans and Zebrafish), and then lead therapeutic regimens showing greatest and most consistent efficacy in these models will be administered in an individualized (‘n-of-1’) treatment trial to objectively evaluate outcomes on overall well-being and organ-specific function in a child with OPA1 disease and other mitochondrial disorders. This personalized therapeutic development approach will enable future clinical trials for assessing therapies that prove effective in patients having similar molecular genetic causes or types of biochemical deficiencies underlying their mitochondrial discasc.

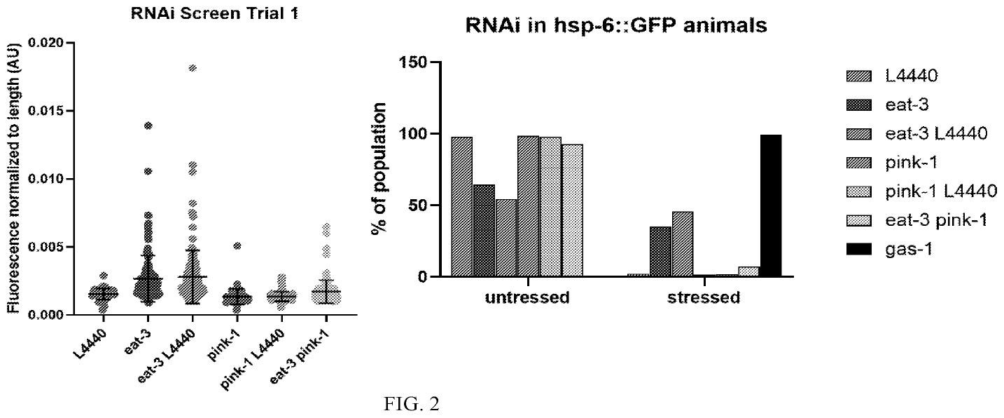

FIG. 2 shows that inhibition of pink-1 using RNAi reduces mitochondrial stress in an eat-3 knockdown model of OPA1 disease. Wild-type animals were treated with different RNAi constructs to knock down expression of genes of interest. Knockdown with eat-3 is the disease model in this case. Pink-1 is a mitophagy gene and pink-1 knockdown in the context of eat-3 knockdown rescues the mitochondrial stress assessed by quantitation of hsp-6p green fluorescence.

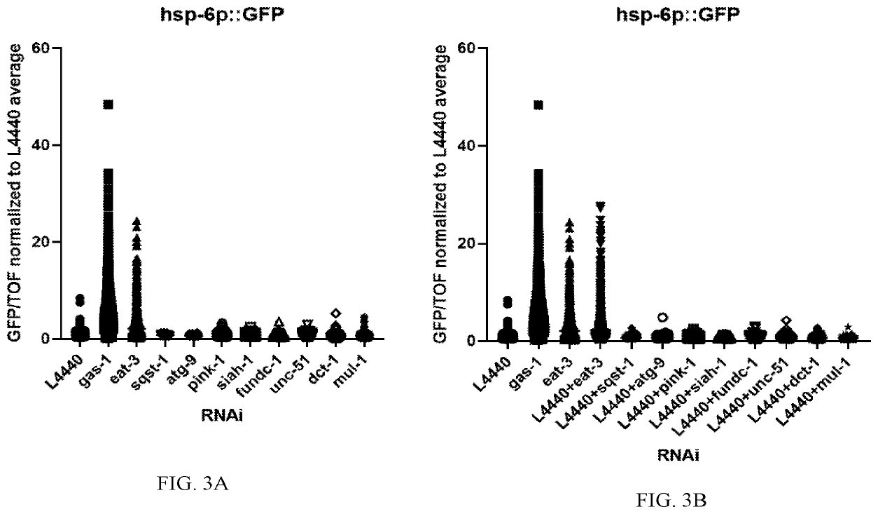

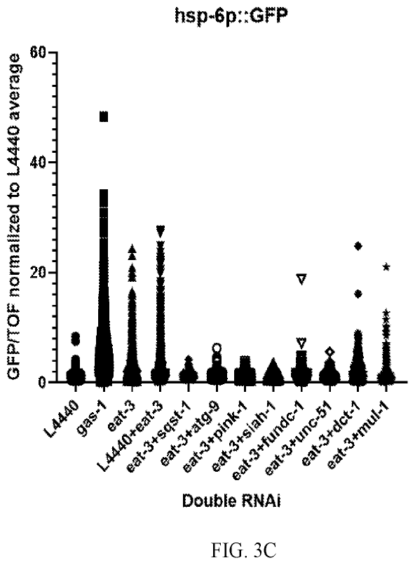

FIGS. 3A-3C show the results of RNAi silencing of 8 different mitophagy modulators, which effectively reduces mitochondrial stress in eat-3 knockdown model. FIG. 3A shows that knock down in wild-type worms with either gas-1 or eat-3 RNAi induces mitochondrial stress but RNAi knockdown with 8 different mitophagy genes does not. FIG. 3B shows that half dose of eat-3 RNAi induces equivalent mitochondrial stress as full-dose eat-3 RNAi. FIG. 3C shows that knock down of 8 different mitophagy modulator genes with RNAi reduces mitochondrial stress in eat-3 RNAi treated wildtype animals. Enhanced reduction of stress obtained with certain combinations of RNAi is shown. These represent specific gene targets within the mitophagy pathway to therapeutically inhibit (by genetic or pharmacologic means) to improve health in OPA1 disease.

The results from screening assays assessing the effects of mitophagy modulating agents on thrashing in eat-3(R289Q) animals are shown in FIGS. 4A, 4B and 4C. FIG. 4A shows that Folinic Acid, LicCl, Metformin, N-acetylcysteine, Nicotinamide, and Resveratrol rescue the eat-3 (R289Q) worms' thrashing behavior defect. FIG. 4B-4C shows that known mitophagy pathway modulating compounds Valproic Acid, Dexamethasone, Etoposide, Vorinostat, and Quercetin each significantly rescues the eat-3(R289Q) worms' thrashing defect.

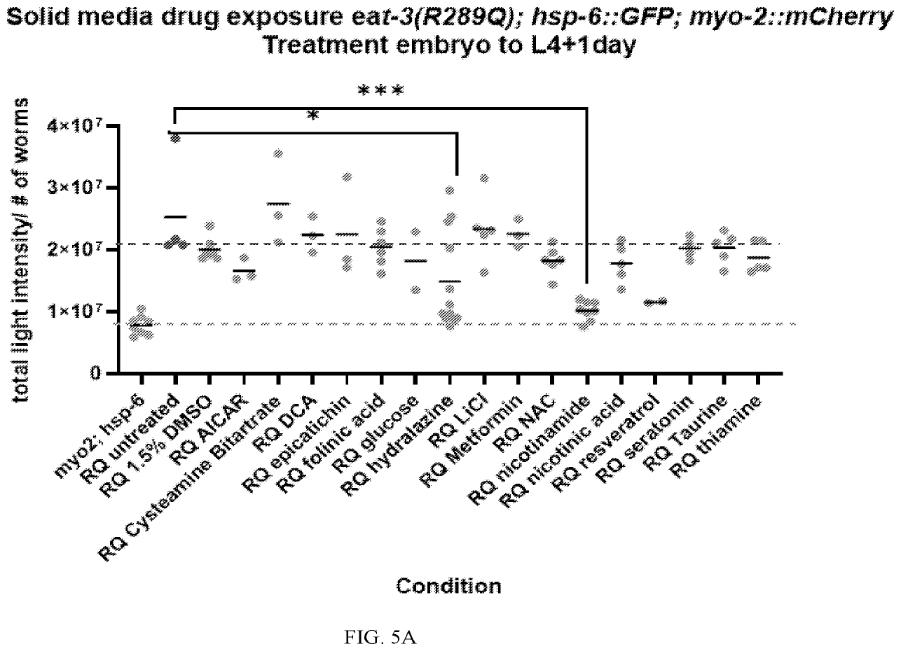

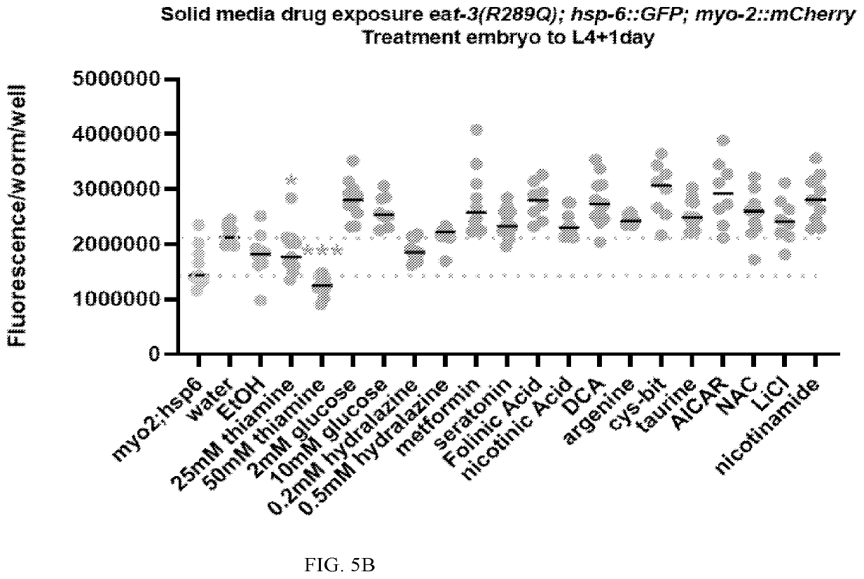

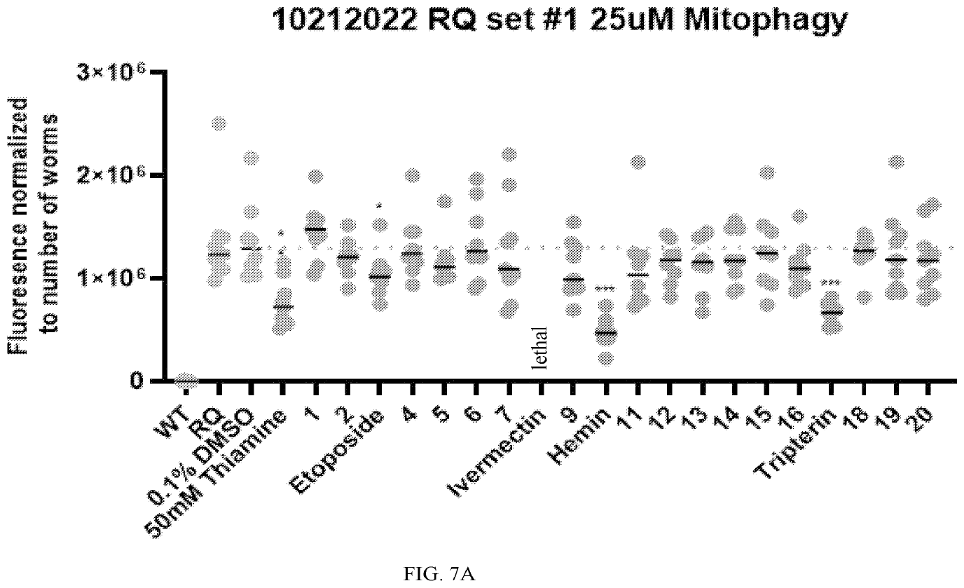

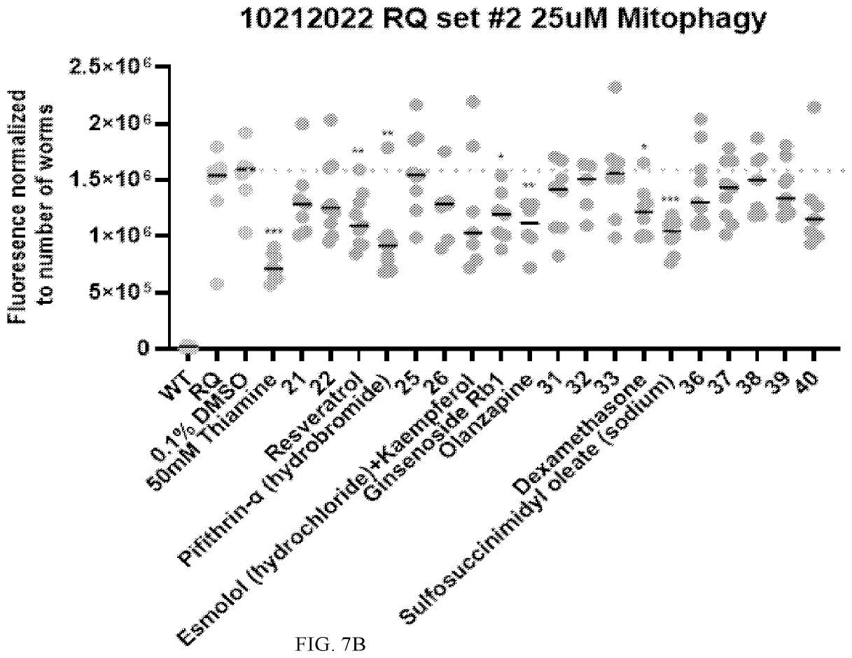

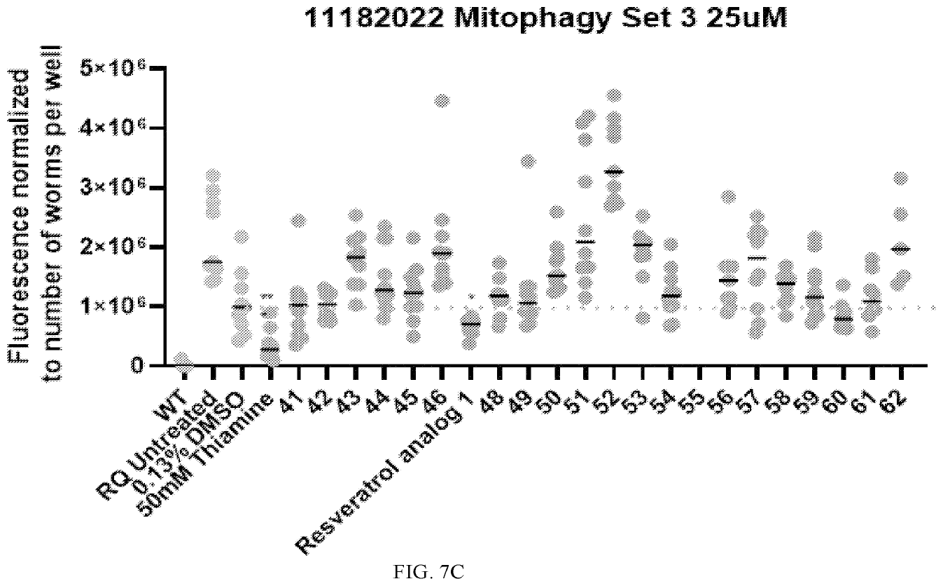

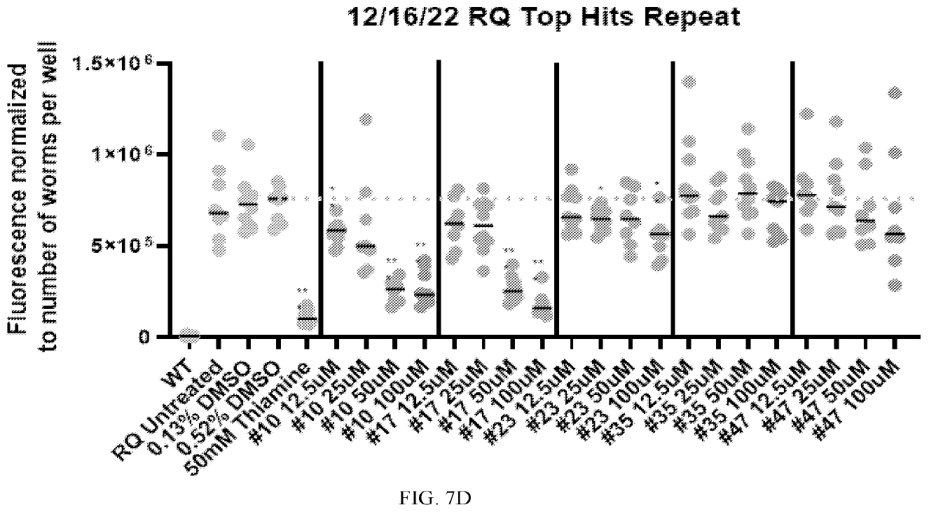

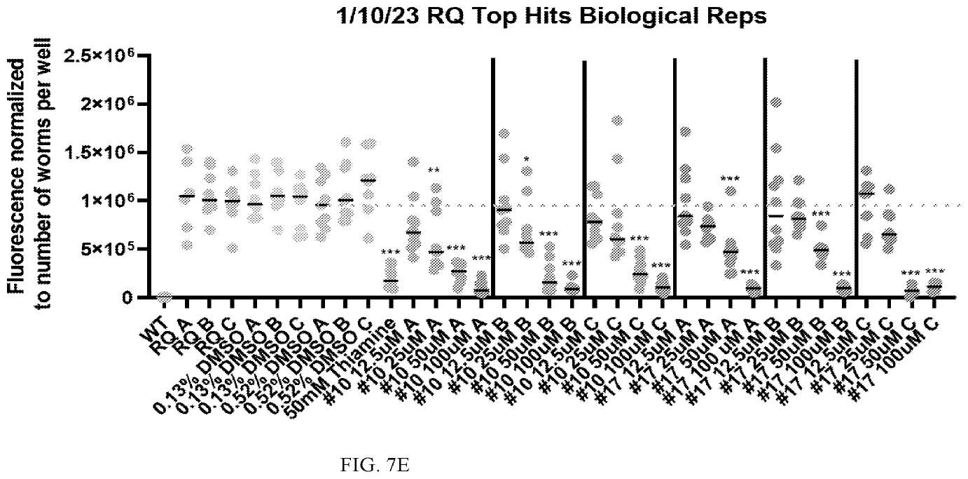

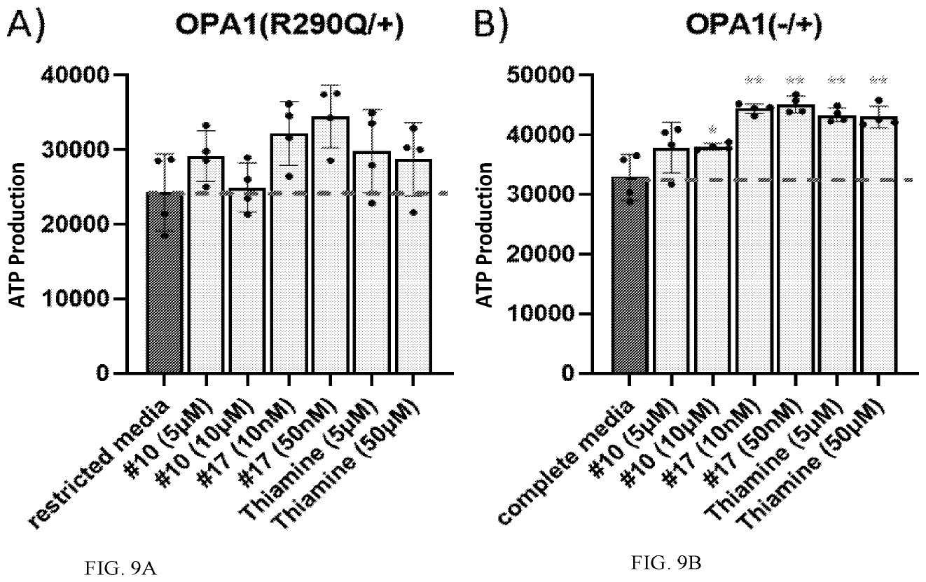

The results from additional screening assays show the effects on mitochondrial stress in eat-3(R289Q) animals of candidate mitochondrial disease therapies when exposed to worms grown on solid media plates. FIG. 5A shows that hydralazine and nicotinamide significantly rescue mitochondrial stress in eat-3(R289Q) animals. FIG. 5B shows that 25 mM Thiamine, and 50 mM Thiamine significantly rescue mitochondrial stress in eat-3(R289Q) animals. Treatment is applied from embryo to L4+1 day in FIGS. 5A-5B. Additionally. the mitophagy modulating compounds tested in eat-3(R289Q) animals assaying for mitochondrial stress response identified multiple hits (FIG. 7A-7E). Most interestingly, Hemin and Tripterin was shown to be able to reproducibly rescue mitochondrial stress response in eat-3(R289Q) animals in a dose-dependent manner. Furthermore, Hemin, Tripterin and Thiamine treatment improved ATP production in fibroblasts derived from patients carrying (R290Q/+) and (1403T/+) mutations (FIG. 9A-9B).

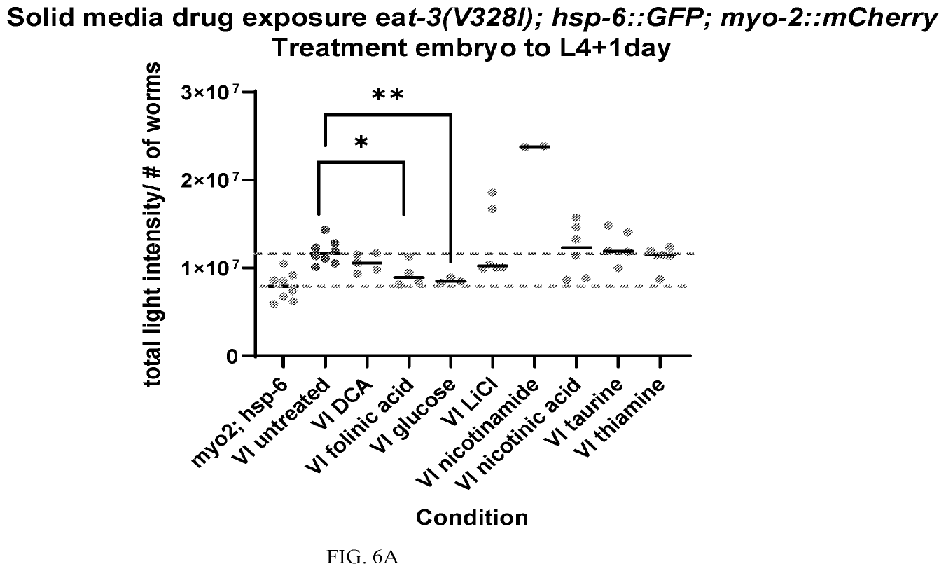

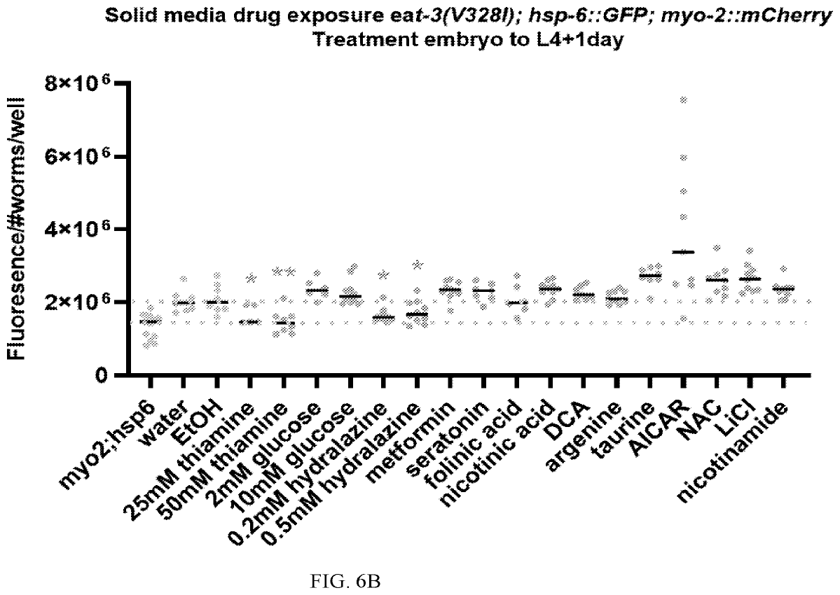

FIGS. 6A-6B shows the results of screening assays on mitochondrial stress in another humanized OPA1 disease allele model we generated, eat-3(V328I) animals, of candidate mitochondrial disease therapies when exposed to worms grown on solid media plates. FIG. 6A shows that folinic acid and glucose significantly rescue mitochondrial stress in eat-3(V328I) animals. FIG. 6B shows that 25 mM Thiamine, 50 mM Thiamine, 0.2 mM Hydralazine, 0.5 mM Hydralazine significantly rescue mitochondrial stress in eat-3(V328I) animals. Treatment is applied from embryo to L4+1 day adult stage in FIGS. 6A-6B. FIG. 7 provide a table summarizing the significant treatment results of candidate mitochondrial disease therapies on mitochondrial stress reduction in both eat-3 mutant alleles (R289Q and V328I) strains that were shown in FIGS. 4-6.

### REFERENCES FOR EXAMPLE I

[1] M. J. Falk, E. Polyak, Z. Zhang, M. Peng, R. King, J. S. Maltzman, E. Okwuego, O. Horyn, E. Nakamaru-Ogiso, J. Ostrovsky, L. X. Xie, J. Y. Chen, B. Marbois, I. Nissim, C. F. Clarke, D. L. Gasser, Probucol ameliorates renal and metabolic sequelae of primary CoQ deficiency in Pdss2 mutant mice, EMBO Mol Med, 3 (2011) 410-427.
[2] S. McCormack, E. Polyak, J. Ostrovsky, S. D. Dingley, M. Rao, Y. J. Kwon, R. Xiao, Z. Zhang, E. Nakamaru-Ogiso, M. J. Falk, Pharmacologic targeting of sirtuin and PPAR signaling improves longevity and mitochondrial physiology in respiratory chain complex I mutant Caenorhabditis elegans, Mitochondrion, 22 (2015) 45-59.
[3] M. Peng, J. Ostrovsky, Y. J. Kwon, E. Polyak, J. Licata, M. Tsukikawa, E. Marty, J. Thomas, C. A. Felix, R. Xiao, Z. Zhang, D. L. Gasser, Y. Argon, M. J. Falk, Inhibiting cytosolic translation and autophagy improves health in mitochondrial disease, Hum Mol Genet, 24 (2015) 4829-4847.
[4] S. Parikh, R. P. Saneto, M. J. Falk, I. Anselm, B. H. Cohen, R. H. Haas, A modern approach to the treatment of mitochondrial disease, Current Treatment Options in Neurology, in press (2009).
[5] Z. Zhang, M. Tsukikawa, M. Peng, E. Polyak, E. Nakamaru-Ogiso, J. Ostrovsky, S. McCormack, E. Place, C. Clarke, G. Reiner, E. McCormick, E. Rappaport, R. Haas, J. A. Baur, M. J. Falk, Primary respiratory chain disease causes tissue-specific dysregulation of the global transcriptome and nutrient-sensing signaling network, PLOS One, 8 (2013) e69282.

### Example II

Methods for Identification of Mitophagy Modulators for Treatment of Heteroplasmic Single Large-Scale mtDNA Deletion Syndromes (SLSMDS)

The mitochondrial genome (mtDNA) plays a key role in organismal survival. Pathogenic variants in mtDNA, such as single large scale mitochondrial DNA deletions (SLSMD), negatively affect organismal fitness. The severity of fitness defects depends on the frequency of mtDNA genomes that carry a SLSMD, defined as the SLSMD heteroplasmy level. Classic pediatric SLSMD disorders include Pearson's Syndrome (PS) and Kearns-Sayre Syndrome (KSS), although the path to diagnosis may be difficult because the molecular progression of disease in SLSMDS is not well understood. Adult-onset SLSMD disorders include Chronic Progressive External Ophthalmoplegia (CPEO), and CPEO-plus that also has multi-system involvement. Furthermore, no effective therapies or cures exist for SLSMD disorders. One major roadblock to therapeutic discovery is the lack of SLSMD animal models for research analysis since modifying the mitochondrial genome remains challenging and SLSMD have often been unstable when generated, not sustained between generations to permit research study.

The uaDf5 worm strain in C. elegans is an invertebrate animal model that harbors a heteroplasmic 3.1 kb mtDNA deletion, involving 7 tRNAs and 4 proteins. This SLSMD worm model enables basic and translational research investigations into disease progression and therapy development. Mechanisms involved in mtDNA homeostasis are attractive therapeutic targets for SLSMD disorders, including mitophagy that is a cellular process via which dysfunctional mitochondria are degraded via autophagy. The capacity of mitophagy to regulate mtDNA homeostasis is being tested in this model by knocking down key mitophagy pathway components. SLSMD therapy development is also pursued by screening drug libraries in this model, with validation studies planned in SLSMD human patient fibroblasts.

Mitochondria are specialized organelles that carry their own genome encoding 22 tRNA, 2 rRNA, and 13 protein products that participate within the respiratory chain in the production of cellular energy in the chemical form of adenosine triphosphate (ATP). Since all mtDNA gene products are essential for energy production, maintaining the integrity of the mitochondrial genome (mtDNA) is essential for human health. In fact, aberrations in the mitochondrial genome leading to mitochondrial dysfunction, can result in a wide variety of neuromuscular, multi-system, and metabolic disorders. At the cellular level, mitochondrial dysfunction is dealt with by upregulating mitophagy, a mechanism by which dysfunctional mitochondria are tagged and targeted for degradation via the autophagic machinery. During this process, the impact that mitophagy has on the maintenance of mtDNA integrity remains unclear. Our published data show that deleting the mitophagy activator PARKIN (pdr-1^{gk448}) in mutant worms that have an error-prone mtDNA polymerase (POLG), result in a 5-fold increase in mtDNA mutation frequency. This suggests that mitophagy is involved in selective removal of mutant mtDNA genomes. The deletion of PARKIN also increases the mtDNA content by 25% in the POLG mutant animals, suggesting that mitophagy reduces mtDNA copy number. These studies with the POLG mutant C. elegans model containing mtDNA point mutations and mtDNA depletion clearly show a role for mitophagy in mtDNA maintenance. To study the effects of modulating mitophagy on mutant mtDNA harboring a large deletion, we will use the uaDf5 C. elegans strain that stably carries a mixture of mtDNA that are wild-type (WT) or have a 3.1 kilobase (kb) deletion²⁻⁴. The data obtained to date show 1) increasing mitophagy can ameliorate disease symptoms arising from mtDNA genomes with large deletions; 2) mitophagy modulator therapies screened in C. elegans has identified therapies useful for treatment of mtDNA diseases, and 3) efficacious therapies in C. elegans model can be further validated in human patient cell lines. The worm C. elegans is an ideal translational model for these purposes. They have a 30 day lifespan, high fecundity and rapid sexual cycle. It is important to note that while worms are microscopic and invertebrate, they have the cell-types typically affected by mitochondrial diseases, such as neurons and muscle cells. The fitness of the animals can be monitored readily using various locomotive neuromuscular assays that correlate with disease progression. Since worms are easy to grow, amenable to genetic and pharmaceutical treatments, and their fitness can be measured readily, they are well-suited for mechanistic dissection and for screening potential therapies.

To repress mitophagy, RNA interference (RNAi) can be employed to individually knock-down 10 key components of either PARKIN-dependent and/or PARKIN-independent mechanisms of mitophagy⁵ that are homologous between C. elegans and humans. The uaDf5 animals are grown on bacteria expressing the double-stranded RNA corresponding to the gene of interest⁶, which when eaten by the uaDf5 animals, elicit an RNAi response to knock down expression of the gene of interest. Drugs known to upregulate mitophagy can be added to the plates on which uaDf5 animals are cultured. The effects on mitophagy conferred by either drug or RNAi treatment can be quantified by utilizing a worm strain with GFP fused to the mitochondrial protein tomm-20 and RFP fused to the autophagy protein lgg-1⁷. The level of mitophagy is quantified by visualizing GFP and RFP signal overlap by confocal microscopy. Sec FIG. 1. Such experiments enable confirmation that any effects that the RNAi and drugs treatments have on mtDNA integrity, or animal fitness can be confidently attributed to the modulation of mitophagy.

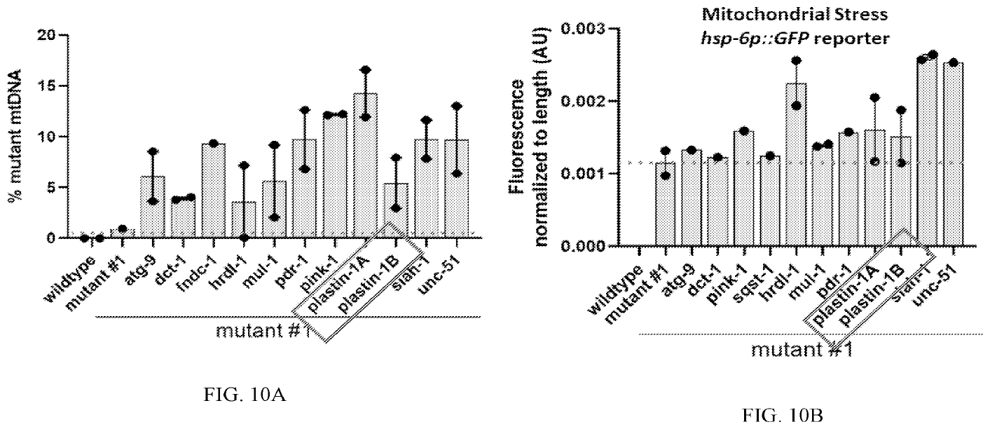

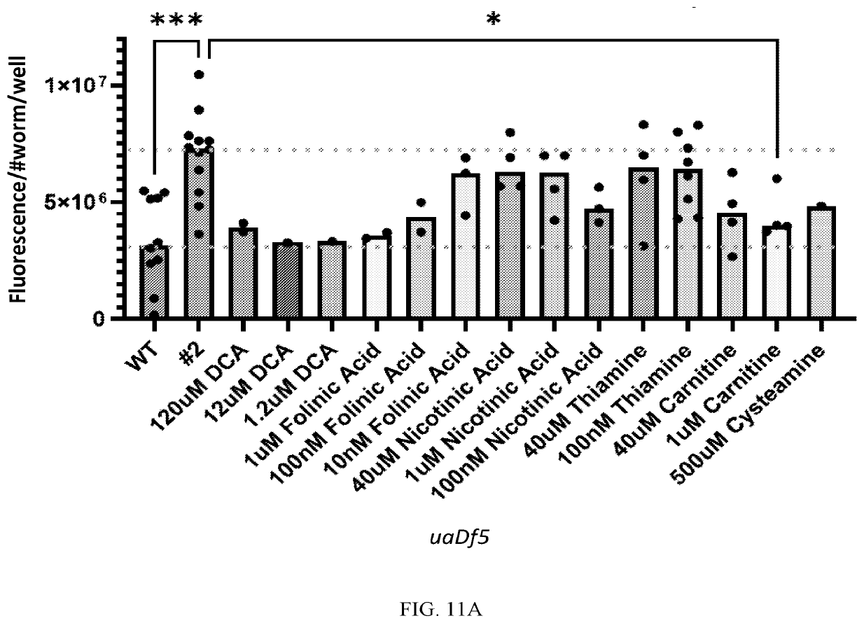

FIGS. 10A-10B show that downregulating mitophagy does increase uaDf5 heteroplasmy and increases mitochondrial stress. FIG. 10A depicts results from an RNAi screen of mitophagy genes on % mtDNA deletion (mtDNAA) heteroplasmy level as assessed in 3 biological replicate experiments of untreated wildtype (hsp-6p::GFP), untreated uaDf5;hsp-6p::GFP #1, and uaDf5;hsp-6p::GFP #1 treated with RNAi against one of 10 mitophagy pathway genes (atg-9, dct-1, pink-1, sqst-1, hrdl-1, mul-1, pdr-1, plastin-1, siah-1, and unc-51). FIG. 10B shows results from an RNAi screen of mitophagy genes on mitochondrial stress assessed in 3 replicate experiment of untreated wildtype (hsp-6p::GFP), untreated uaDf5;hsp-6p::GFP #1, and uaDf5;hsp-6p::GFP #1 treated with RNAi against various mitophagy genes (atg-9, dct-1, pink-1, sqst-1, hrdl-1, mul-1, pdr-1, plastin-1, siah-1, unc-51).

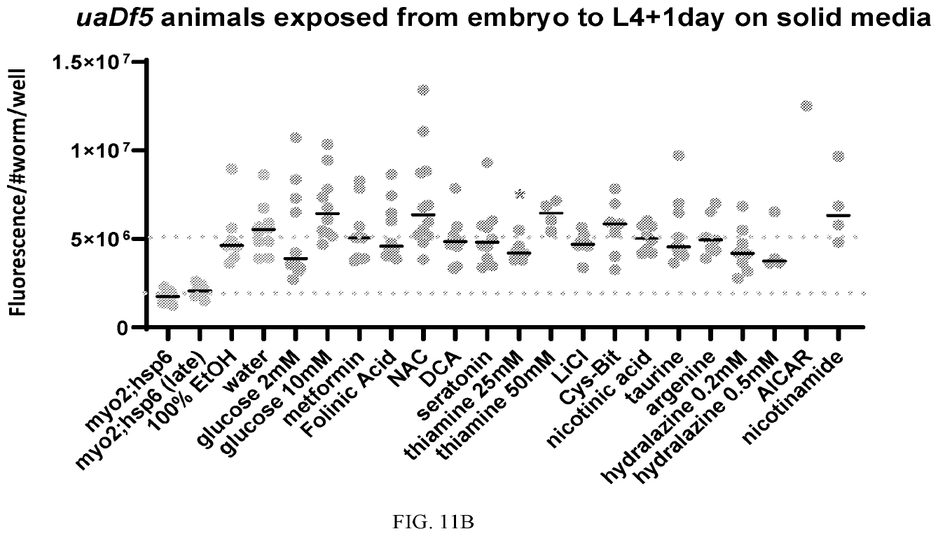

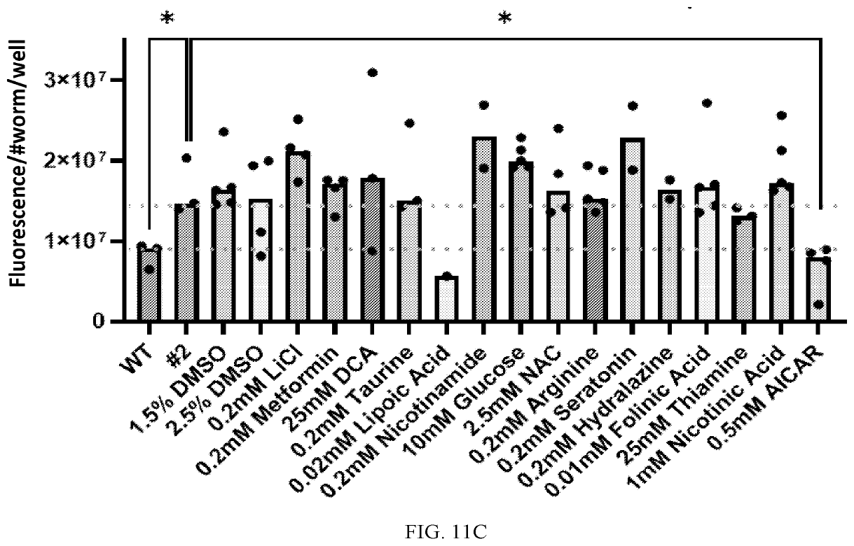

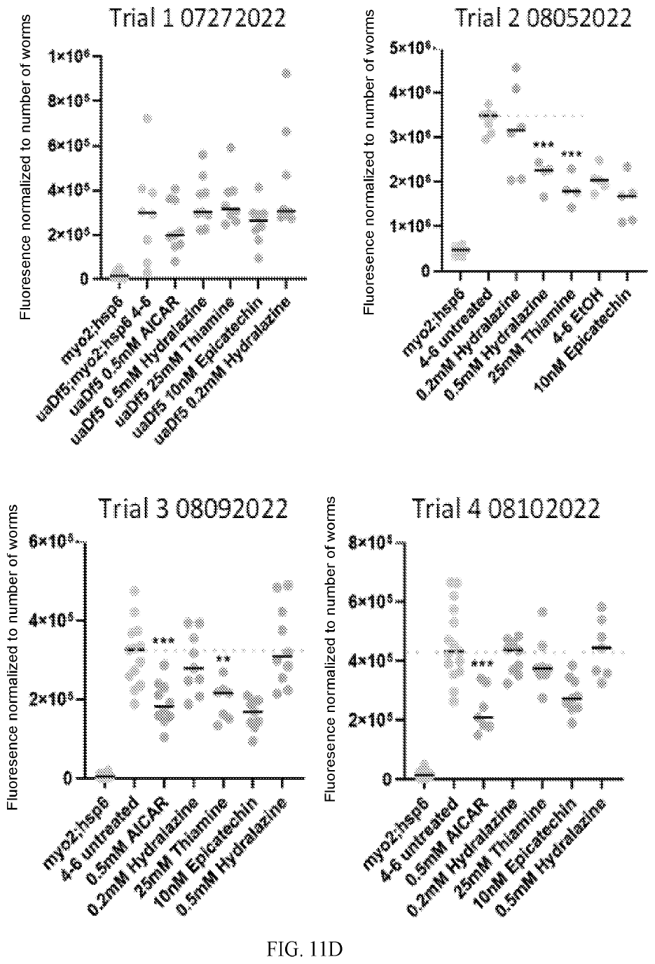

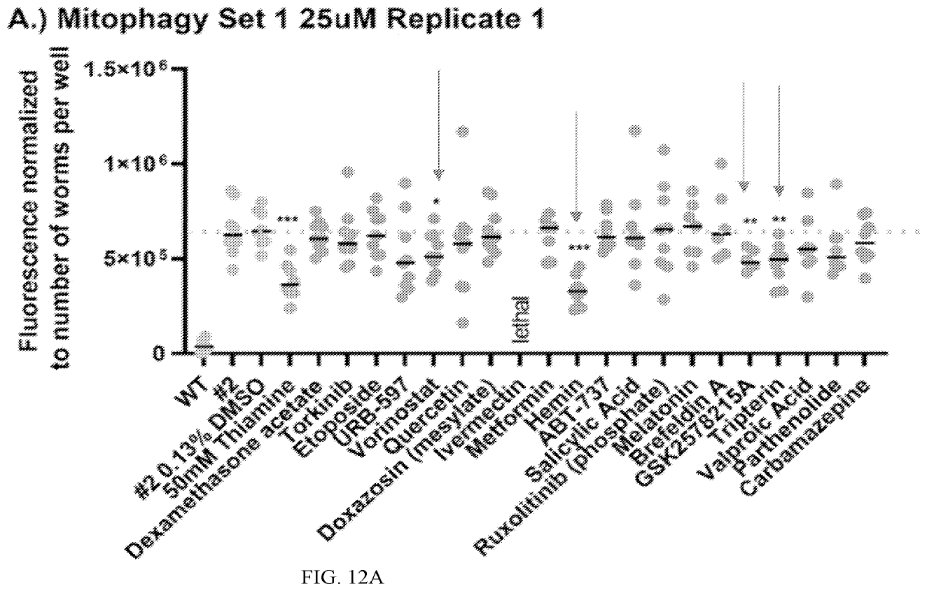

In additional experiments, agents known to benefit mitochondrial respiratory chain complex I disease models in our research laboratory were assessed in uaDf5 animals with heteroplasmic SLSMDs. FIG. 11A shows that 40 μM carnitine significantly rescues mitochondrial stress in uaDf5 animals. Dichloroacetate (DCA) and higher doses of folinic acid demonstrate strong trends in rescuing mitochondrial stress in uaDf5 animals. FIG. 11B shows that 25 mM thiamine significantly rescues mitochondrial stress in uaDf5 animals. FIG. 11C shows that AICAR significantly rescues mitochondrial stress in uaDf5 animals. FIG. 11D shows that 0.5 mM Hydralzine, 25 mM Thiamine, 0.5 mM AICAR rescue mitochondrial stress response in uaDf5 animals.

As discussed above, the uaDf5;myo-2p::mcherry;hsp-6p::GFP worm (C. elegans) strain #2 with an average of 30% heteroplasmy can be used to advantage in mitochondrial unfolded protein response (UPR^{mt}) stress reporter assays. Under normal conditions the mutant worms have a high level of mitochondrial stress assessed by green fluorescence which facilitates the identification of drugs which lower the fluorescence. We validated drugs which ameliorate complex I disease and identified five conditions that consistently rescued the fluorescent phenotype in the animals. These included 25 mM Thiamine, 50 mM Thiamine, 0.2 mM MitoQ, 0.02 mM Lipoic Acid and 0.5 mM AICAR. We further retested these compounds 24 and 48-hour exposure (data not shown). Duc to 0.5 mM AICAR being diluted in ethanol (EtOH) and the other four compounds being diluted in water, we included both a water treated and EtOH treated control in the assay. We noticed that the EtOH treated animals had much lower fluorescence compared to the water treated due to the effect of EtOH on the mitochondrial stress reporter expressions. We removed 0.5 mM AICAR as a test compound since the solvent effects confounds the interpretation. The level of rescue was not significantly different between the 24-hour versus the 48-hour exposure. Therefore, we decided to use 24-hour exposure for speed, and we used 50 mM Thiamine as the positive control.

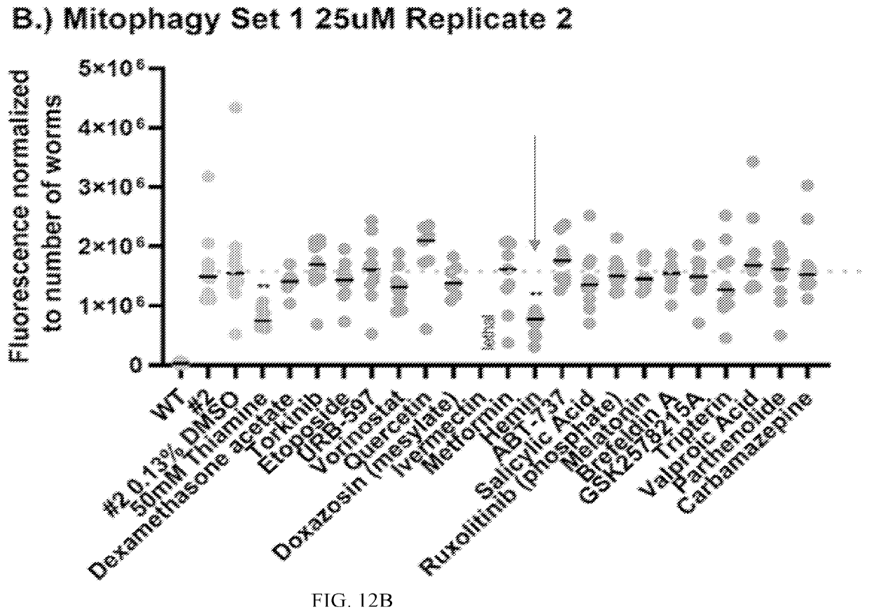

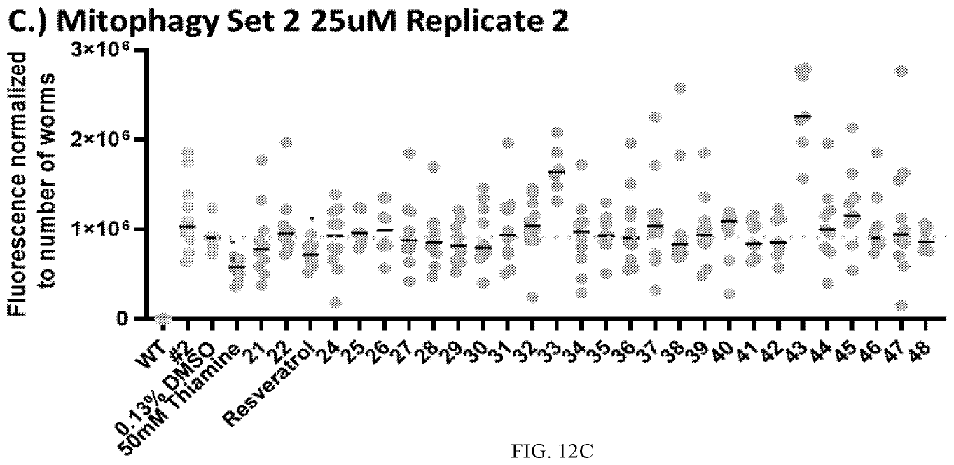

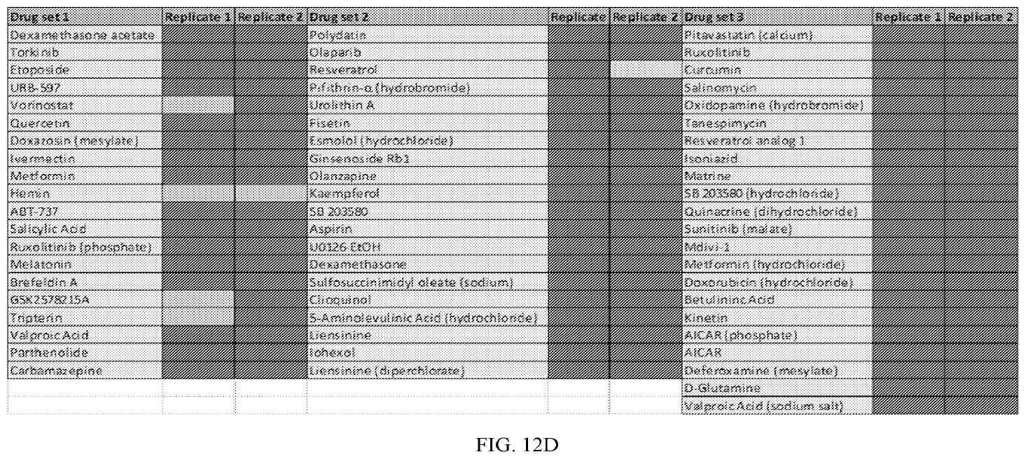

We screened the 62 mitophagy modulating compound library in using 50 mM Thiamine as a positive control in uaDf5 strain #2 (FIG. 12A-12D). The screen was divided into three different sets. The first 20 compounds in the library being “set 1”, the next 20 being “set 2” and the last 22 being “set 3”. The entire library was tested twice at the concentration of 25 μM to identify potential hits, which were also tested with a 4-dose range to identify best dose for use. Five compounds were able to significantly rescue the mitochondrial stress: Vorinostat, Hemin, GSK2578215A, Tripterin and Resveratrol (FIG. 12D).

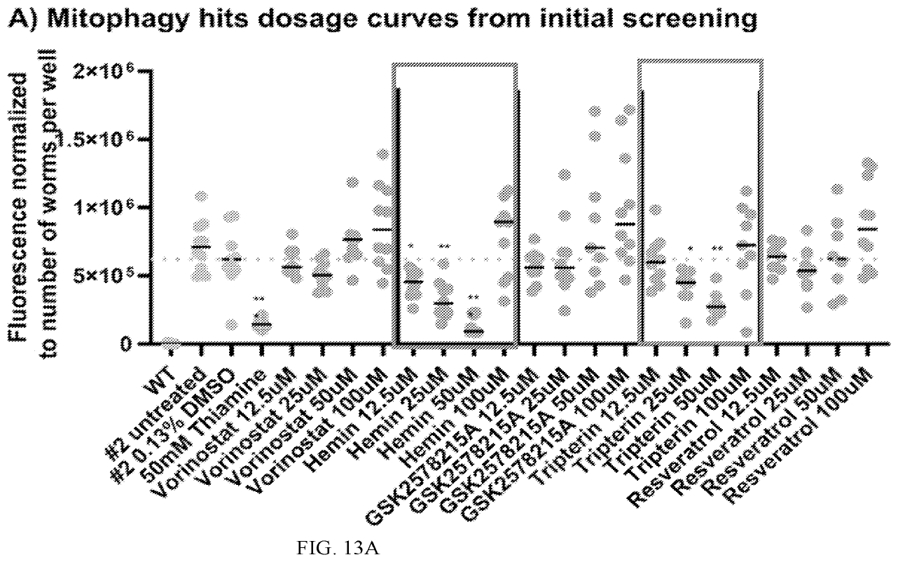

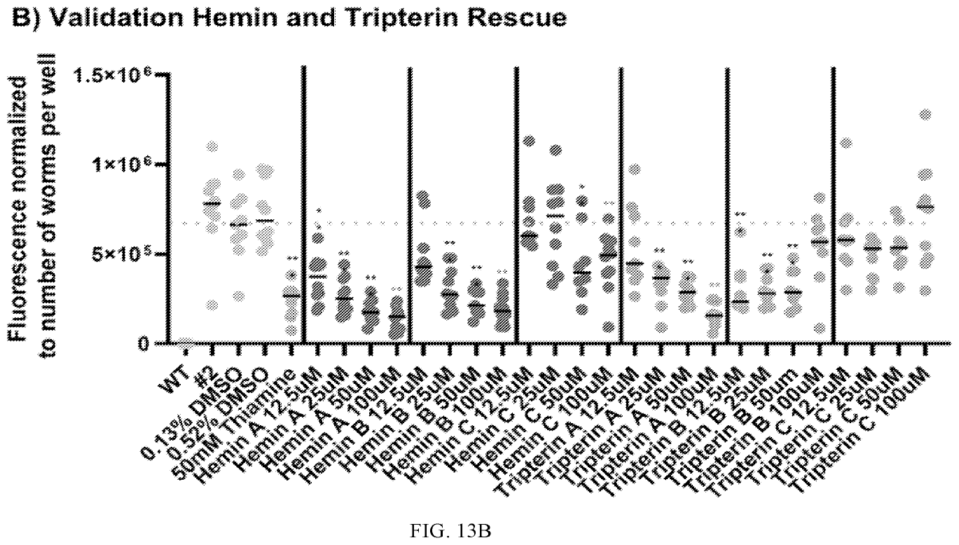

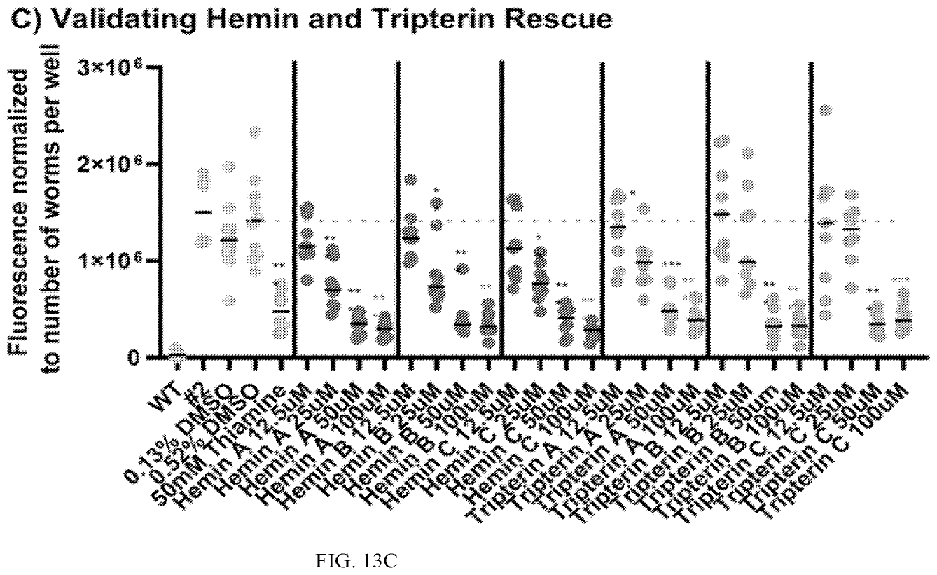

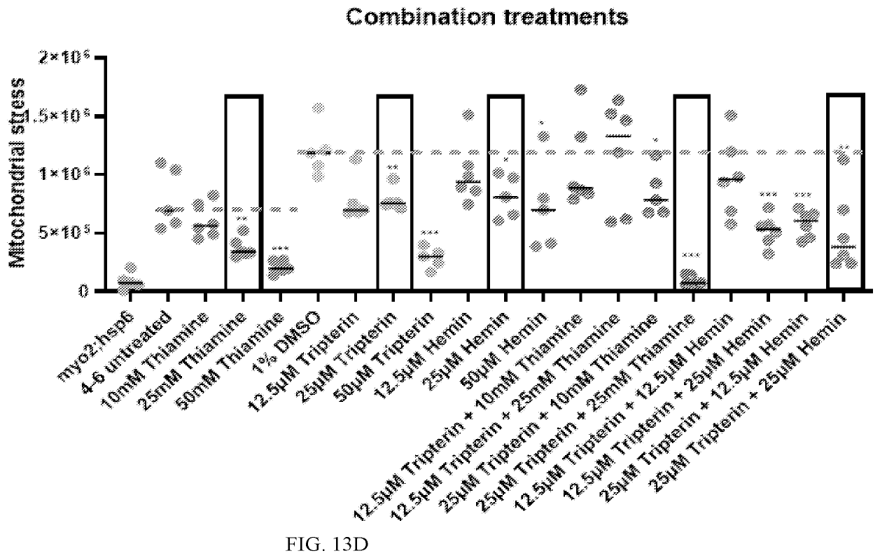

Validating 5 the hits identified from the mitophagy modulating library: Vorinostat, Hemin, GSK2578215A, Tripterin and Resveratrol were each tested at 12.5 μM, 25M, 50 μM and 100 μM (FIG. 13A). Of these 5 compounds, Hemin and Tripterin reproducibly rescued the mitochondrial stress response in a dose dependent manner (FIG. 13A-13C). Replicate “C” in FIG. 13B had no effect but an additional 3 biological replicates (FIG. 13C) show a dose dependent response of Hemin and Tripterin. We did notice that at the highest concentration (100 μM), both compounds appeared to have a detrimental impact on the animals, with at least half of the animals in those wells being dead after the 24-hour exposure. Quantifying the population health of these animals was assessed by performing wormscan. In short, the worms in the 384 well plate prior to the scan to determine the fluorescence, were scanned twice using brightfield light and movement levels determined. Given that not all the animals were dead, the movement analysis didn't show any detrimental effects of 100 μM on animal fitness. However, careful observations of the fluorescent images show the detrimental effects of the treatments clearly.

### Hemin and Tripterin for Use in Therapies for SLSMD Diseases.

Hemin is derived from the crystallization of heme. It has been approved to treat acute porphyria due to its ability to inhibit porphyrins (Rainforth Collins, 2023). In the perspective of mitophagy, hemin induces mitophagy by decreasing the mitochondrial membrane potential (Higdon et al., 2012). Hemin also promotes autophagy, by upregulating the formation of a marker of autophagy called LC3-II (Higdon et al., 2012).

Tripterin (also known as Celastrol) is classified as a proteasome inhibitor and has been used for treatment for several conditions including inflammatory diseases, cancer, and obesity (Cascão et al., 2017). In studies on the effect of tripterin in human osteosarcoma cells, it was shown to activate autophagy in the ROS/JNK signaling pathways (Lin et al., 2020). In murine models of Parkinson Disease (PD), Tripterin was shown to upregulate two components of mitophagy; PINK-1 which is a serine/threonine-protein kinase and DJ-1 a cytoprotective protein (Lin et al., 2020). Upregulation of PINK-1 and DJ-1 leads to the induction of mitophagy. Most interestingly, 25 μM Tripterin+25 mM Thiamine combination and 25 μM Tripterin+25 μM Hemin combinations show synergistic improved efficacy compared to either treatment alone. (FIG. 13D). This information can be used to advantage for production of efficacious formulations for therapy. Lastly, not only do these therapies reduce mitochondrial stress in the organisms, but the combinations of 50 mM Thiamine, 50 μM Hemin, and 50 μM Tripterin also appear to lower mutant mtDNA heteroplasmy, thereby effectively and synergistically targeting the etiology of SLSMD diseases (FIG. 15).

FIG. 14 summarizing the therapies that significantly rescue mitochondrial stress at the level of hsp-6p fluorescence quantitation of UPR^{mt} induction in uaDf5 animals is shown in FIGS. 11, 12, 13.

### Determining Primers to Test Heteroplasmy Levels in Patient Cell Lines

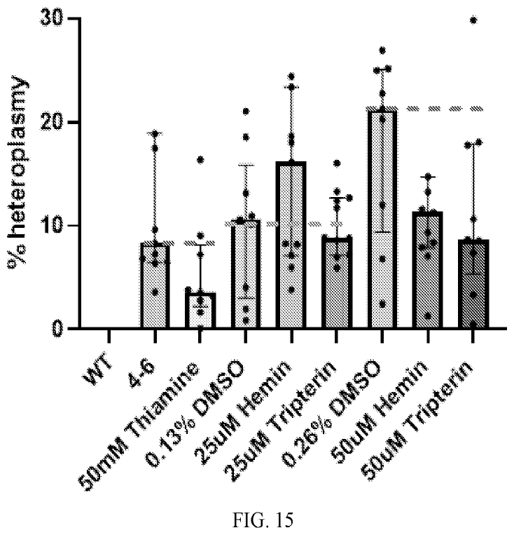

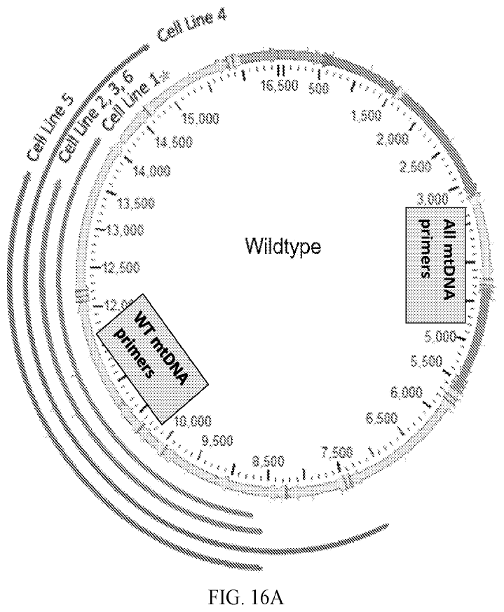

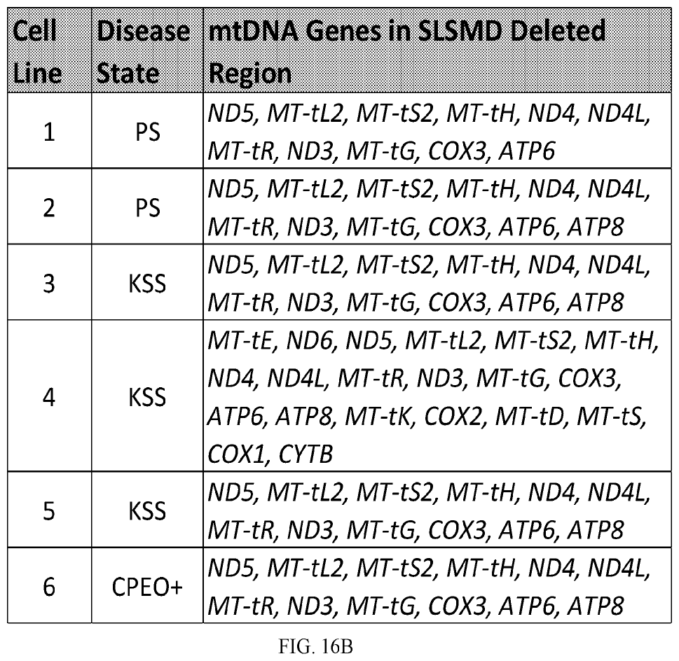

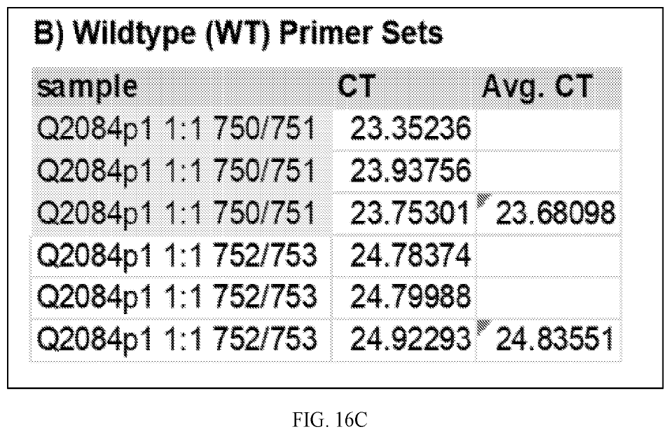

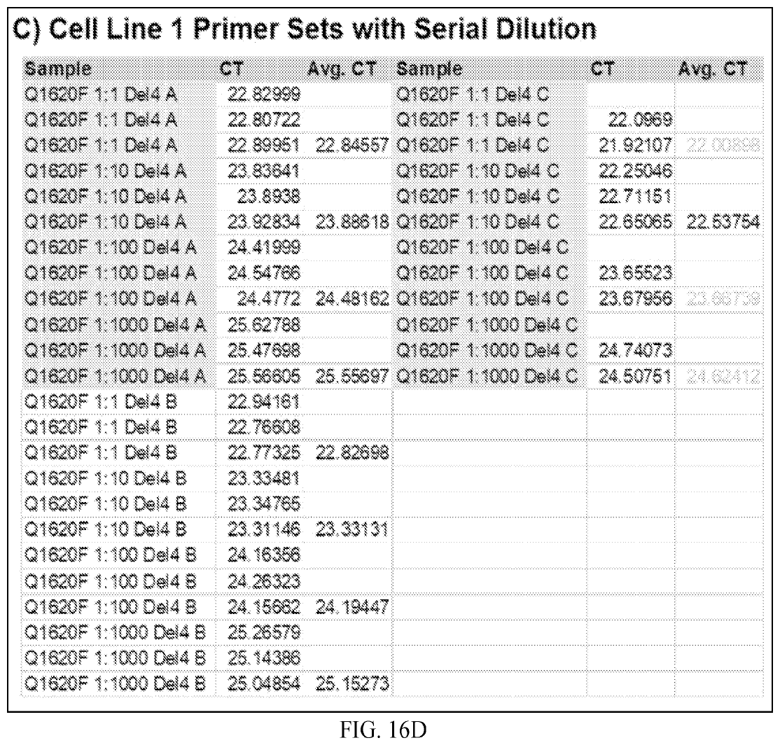

Primers have been designed for each of the unique deletion signatures as well as a primer designed to only amplify wildtype mtDNA (FIGS. 16A and 16B). We hypothesized that fresh cells may work better as the source of DNA for the qPCR. The first test with live cells was done on the wildtype only primer sets at one DNA concentration, which proved successful (FIG. 16C). The 4 unique deletion regions were then tested at 4 DNA concentrations (1:1, 1:10, 1:100 and 1:1000). Only the primers that amplified Deletion 4 (SLSMD of Cell Line #1) were successful (FIG. 16D) (Table 2). The failure to amplify the other SLSMD could be either because, (a) the primers are substandard or (b) the SLSMD heteroplasmy is low. If the latter scenario is true, then we can still use the wildtype mtDNA primers and all mtDNA primers to infer the SLSMD heteroplasmy.=

TABLE 1

| Primer | Sequence | SEQ ID NO: |
|---|---|---|
| HS_mtDNA_Del3F1 | gcccacttccactatgtcct | 1 |
| HS_mtDNA_Del3R1 | gtgagggtgggactgtctac | 2 |
| HS_mtDNA_Del3F2 | tacgttgtagcccacttcca | 3 |
| HS_mtDNA_Del3R2 | gggtgggactgtctactgag | 4 |
| HS_mtDNA_Del3F3 | tgtagcccacttccactatgt | 5 |
| HS_mtDNA_Del3R3 | aggaggtggagtgttgctag | 6 |
| HS_mtDNA_Del2F1 | cagtttcatgcccatcgtcc | 7 |
| HS_mtDNA_Del2R1 | taggctgccaatggtgagg | 8 |
| HS_mtDNA_Del2F2 | cccctctagagcccactgta | 9 |
| HS_mtDNA_Del2R2 | taatgctaggctgccaatgg | 10 |
| HS_mtDNA_Del2F3/DelF2 | acagtgaaatgccccaacta | 11 |
| HS_mtDNA_Del2R3/DelR2 | cagggaggtagcgatgagag | 12 |
| HS_mtDNA_Del1F1 | acaaactaccacctacctccc | 13 |
| HS_mtDNA_Del1R1 | cagggaggtagcgatgagag | 14 |
| HS_mtDNA_Del1F3 | tgccccaactaaatactaccgt | 15 |
| HS_mtDNA_Del1R3 | gctcaggcgtttgtgtatga | 16 |
| HS_mtDNA_Del4F1 | cgccgcagtactgatcattc | 17 |
| HS_mtDNA_Del4R1 | cagggttgattcgggaggat | 18 |
| HS_mtDNA_Del4F2 | ctccctcaccaaagcccata | 19 |
| HS_mtDNA_Del4R2 | gggttaggatgagtgggaaga | 20 |
| HS_mtDNA_Del4F3 | cccacaatcctaggcctacc | 21 |
| HS_mtDNA_Del4R3 | atgaaggagaggggtcaggg | 22 |
| HS_mtDNA_WildtypeF1 | actcacaacaccctaggctc | 23 |
| HS_mtDNA_WildtypeR1 | gcttcgacatgggctttagg | 24 |
| HS_mtDNA_WildtypeF2 | gctcccttcccctactcatc | 25 |
| HS_mtDNA_WildtypeR2 | tgagaatgagtgtgaggcgt | 26 |
| HS_mtDNA_WildtypeF3 | ccttggctatcatcacccga | 27 |
| HS_mtDNA_WildtypeR3 | tcttgggcagtgagagtgag | 28 |

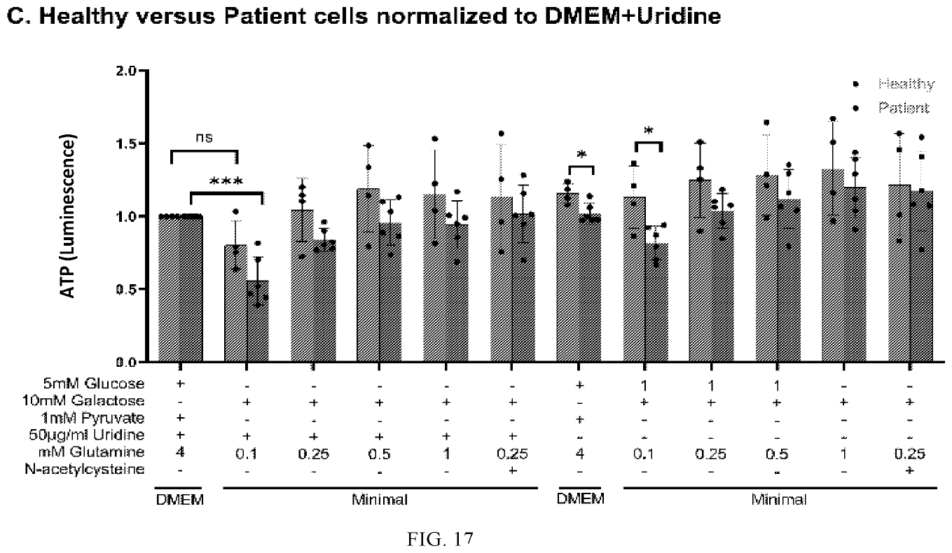

FIG. 17 show the effects of limited nutrient on 4 healthy patient fibroblast (green) and 6 SLSMD patient fibroblast. This shows that the mutant cells are more susceptible to nutrient deprivation that forces mitochondria to work harder. These cells and these nutrient limiting conditions will be used to assess efficacy of therapies for preclinical studies in a human cell model.

To assess the effects of modulating mitophagy on the fitness of uaDf5 animals, we perform thrashing assays following delivery of RNAi (to repress mitophagy), drug (to promote mitophagy), or no treatment and compare them to wild-type (WT) controls^{1,8-10}, as described above in Example I. Thrashing behavior is assessed in 5-day and 10-day old adult uaDf5 and WT worms grown on mitophagy modulating conditions with RNAi, drugs, or no treatment control plates. Thrashing behavior, in body bends per second, is assessed by placing worms in a drop of liquid, video recording, and video analysis with ImageJ analysis¹¹. Three biological replicate experiments with 25 worms per replicate are performed on adult days 5 and 10 in uaDf5 and WT worms. Student's t-test is used to evaluate for statistically significant differences between groups. The mitochondrial mutants typically exhibit reduced thrashing activity at baseline^{1,8-10}. We expect that repressing mitophagy in uaDf5 worms will worsen thrashing activity, while promoting mitophagy will improve thrashing in the uaDf5 mtDNA deletion worms closer to that of wild-type worm activity levels. These experiments assessing the fitness of the animals are important as understanding whether mitophagy modulation in any specific genetic condition can reverse the signs and symptoms of the disease because for human patients, it is critically important to regain their health and fitness.

Chemical modulators of mitophagy may be utilized as mtDNA disease therapies. Conducting a drug screen will be essential for rapid identification and prioritization of efficacious treatments for mtDNA genome deletion diseases. An effective screen will require (i) an outcome that exhibits a defect in the mutant animals below 50% of the wild-type phenotype, (ii) a positive control drug that rescues the phenotype of the mutant animals to at least 75% of wild-type levels, and (ii) a negative control drug that has no effect or worsens the phenotype of the mutant animals. To identify controls, we will conduct thrashing-based activity assays in uaDf5 animals at baseline and upon treatment with known pharmacologic mitophagy activators (eg, 17-AAG, Urolithin A, and Metformin) and inhibitors (eg, Mdivi-1). To further convert this thrashing assay to a high-throughput format, we will use a WormCamp-based screening robot developed in our research laboratory in collaboration with Professor Chris Fang-Yen at the University of Pennsylvania¹². With the proper controls in place and the screen developed, we will first screen 50 FDA-approved drugs known to regulate mitophagy to increase the chances of identifying potential therapies in the uaDf5 animals. The screen will subsequently be expanded to test a library of ˜2,400 FDA-approved drug library (MicroSource Library). By repurposing FDA-approved therapies, we will expedite the process for use of these drugs in patients. Drugs that improve the thrashing defect in uaDf5 animals by at least 50% will be identified as hits, and these hits will be pursued to further validate their efficacy in mtDNA deletion disease models in human patient cell lines.

The therapies identified in C. elegans. can be applied to advantage in cell line models of mtDNA deletion disease. Therapeutic drugs identified in the screens with C. elegans can be validated in patient-derived cell lines of Kearns-Sayre Syndrome (KSS) that are available in our research laboratory and/or also from the Coriell Institute cell repository using cellular ATP production or survival as a readout. Each drug will be tested three times to validate its potency. The top 3 performing drugs validated in the cell line can be further assessed in their ability to improve mitochondrial fitness. We will use commercially available fluorescent-based kits to assay ATP production and mitochondrial membrane potential. Furthermore, we will dissect the impact of the therapy by performing biochemical assays developed in-house to assess the activity of each mitochondrial respiratory chain complex, redox state, lactic acid production and NADH/NAD+ ratio. Each assay will be performed at least 3 separate times on wildtype and patient derived cell lines with or without drug treatment. Student t-test analysis will be performed to assess the significance of any difference changes between groups. These studies will give us insights into the mechanism of action of the therapeutic compound, which can lead to more tailored therapeutic design that could include drug cocktail therapy.

### REFERENCES FOR EXAMPLE II

1—Haroon S, et al. Cell Rep. March 20; 22(12): 3115-3125 (2018)
2—Lin Y F, et al. Nature. May; 533(7603): 416-419 (2016)
3—Tsang W Y & Lemire B D. Biochem Cell Biol. 80(5):645-654 (2002)
4—Liau W S, et al. BMC Genet. March; 8:8 (2007)
5—Kamath R S, et al. Nature. 421, 231-237 (2003)
6—Pickles S, et al. Curr Biol. February; 28(4): R170—R185. (2018)
7—Charmpilas N, et al. Methods Mol Biol. 1759:151-160 (2018)
8—Chaudhari S N, & Kipreos E T. Nat Commun. August: 8, 182 (2017)
9—Hewitt J E, et al. Dis Model Mech. December; 11(12) (2018)
10—Byrne J J, et al. Cell Mol Life Sci. March; 76, 1967-1985 (2019)
11—Nussbaum-Krammer C I, et al. J. Vis. Exp. January; (95) e52321 (2015)
12—Churgin M A, et al. eLife 6, May; 6:e26652 (2017)
13—Campbell J M, et al. bioRxiv, March; 287342 (2018)

### Example III

### In Vivo Visualization of Mitochondrial Genome SLSMD Heteroplasmy Levels in a Sequence Specific Manner

In the present example, the first reported tool designed for in vivo observation of C. elegans mutant versus wild-type mtDNA dynamics in different tissue types simultaneously is described. As noted above, mitochondria are highly specialized cellular compartments with their own genome (mtDNA) in order to streamline the production of energy vital for sustaining life. This specialized function requires careful mtDNA maintenance in order to avoid disruption in organismal bioenergetics. Understanding how healthy cells maintain mtDNA fidelity is crucial for tackling mtDNA-based diseases. There are no FDA approved cures or treatments available for any mitochondrial diseases. Recent estimates state that a least 1 in 4,400 people of all ages in the United States live with inherited mitochondrial disease and 1,000 to 4,000 children with mitochondrial diseases are born every year. Military service members are at a higher risk for mitochondrial diseases due to the nature of their work environment. For instance, they can (a) be exposed to toxins that result in diseases, (b) incur higher rates of physical injuries that result in mitochondrial dysfunction, and (c) be more vulnerable to triggering genetic disorders due to the physical stresses they endure. These possibilities have been shown to either give rise to mtDNA mutations or place soldiers with inherited mtDNA mutation at higher medical risk. Developing the reporter tool will anchor a deeper understanding of the biological processes involved in removing mutant mtDNA genomes in animals. Identification of these processes will be instrumental for developing therapeutics to eliminate mutant mtDNA genomes in patients with mitochondrial diseases. Mutant mtDNA genomes also acquire in many human patients' tissues with age, causing many of the symptoms of comorbidities of aging; having tools to monitor and reduce mtDNA genome deletions in the aging population can improve survival and healthy aging.

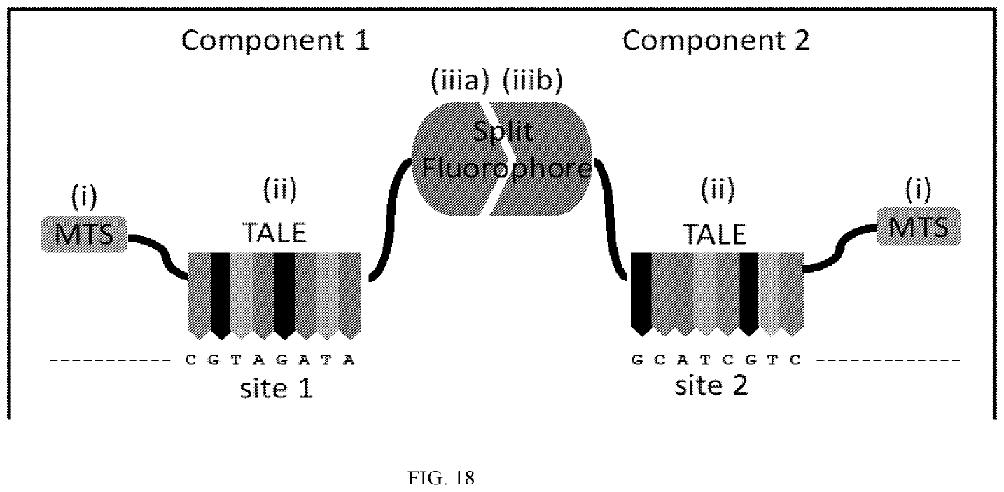

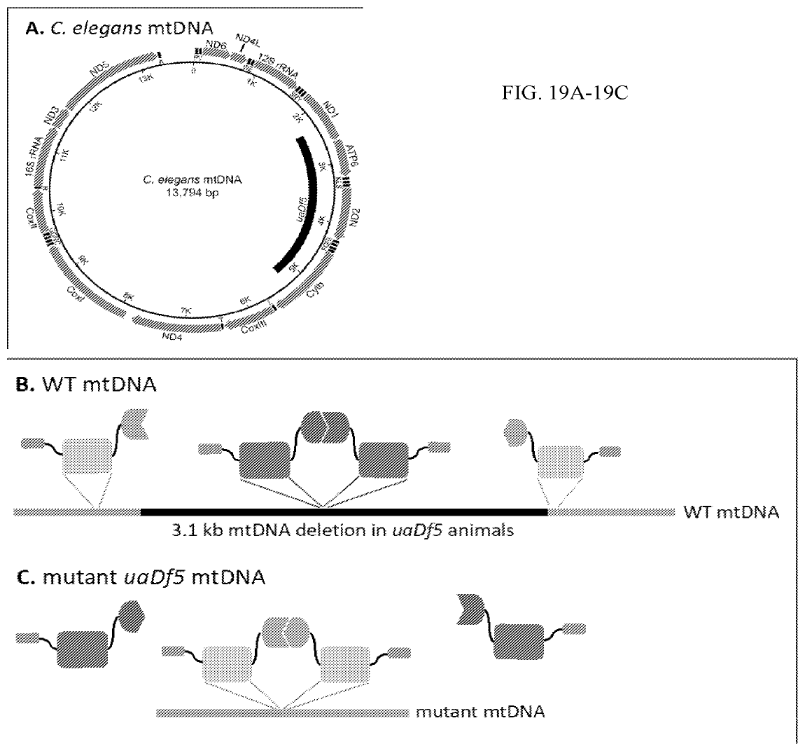

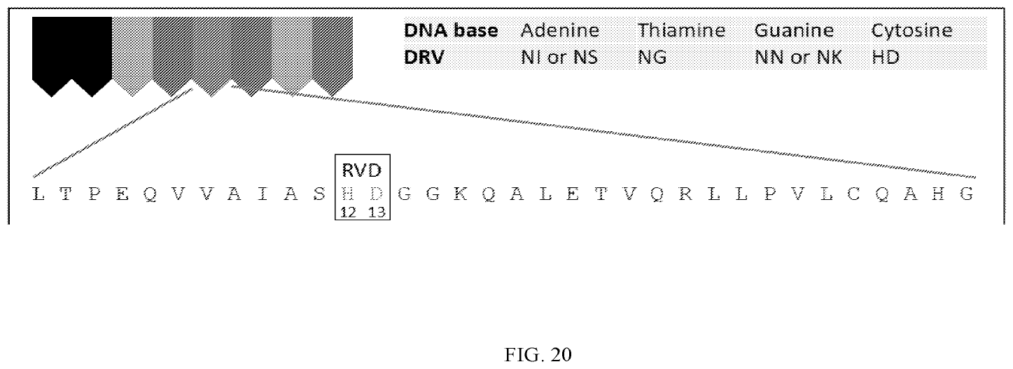

A major roadblock to identifying the mechanisms by which cells clear mutated mtDNA is the inability to separately observe mutant and wild-type mtDNA. To overcome this hurdle, reporter animals can be genetically engineered to enable visualization of both wildtype and mutant mtDNA genomes. Sec FIG. 18. The reporter construct can be generated in C. elegans, a translucent worm species amenable to genetic engineering and visualization by microscopy. The uaDf5 worm strain carries a mixed population of wild-type and mutant mtDNA, and is perfect for tracking two different mtDNA types. See FIG. 19A-19C. The fluorescent reporters will be developed in the uaDf5 C. elegans strain using three specific approaches. 1: Protein constructs that specifically recognize either mutant or wild-type mtDNA will be generated. 2: The optimal fluorescent protein that can function adequately in the mitochondrion will be identified. 3: The DNA recognizing protein and the fluorescent protein will be fused, and subsequently used to engineer uaDf5 worm strains to develop the reporter strains. These reporter strains can then be validated by microscopy. The resulting reporter animals will allow, for the first time, for real time, live observation of mutant versus wild-type mtDNA dynamics in different tissue types simultaneously. This tool will help penetrate a major barrier to identifying mechanisms by which mutant mtDNA genomes are cleared by cells. Indeed, this new tool has enabled the development of new, effective therapies for mtDNA-related mitochondrial diseases as described above and shown in FIG. 13. The development of these much-needed therapeutics will significantly improve the lives of patients currently suffering from mitochondrial disease, including military service members and their families who endure mtDNA diseases, as well as patients of all ages, and the general aging population.

Generation of TALE Pairs that Uniquely Recognize Either Wildtype or Mutant mtDNA in C. elegans.

Dissecting biological processes that help maintain mtDNA integrity will be instrumental in developing therapies for mtDNA diseases. However, such an endeavor is expensive and time-consuming in mammalian organisms and simpler model organisms such as the nematode, C. elegans, offer a practical alternative. C. elegans have neurons and muscle cells that rely on cellular biological processes homologous to those in humans and have highly conserved mtDNA genomes that enable ready modeling of mtDNA diseases. Many of the characteristics that make worms a good model for these studies can also be true for cell culture, such as amenability to genetic engineering, visualization and ease of maintenance. However, the C. elegans model provides the opportunity to study a whole organism with different tissues and organ systems simultaneously. Considering that mtDNA maintenance and mitochondrial dynamics differ between different tissues, the ability to study the whole organism is an important advantage. Therefore, the reporter tool will ultimately be constructed in a C. elegans strain that stably carries a mixed mtDNA genome population of wild-type (WT) mtDNA and mutant mtDNA that contains a deletion (FIG. 18).

Several DNA sequence recognition tools have been developed in the context of genome engineering, and the most common tools are based on Transcription Activator-Like Effectors (TALE), Zinc-Fingers and CRISPR-Cas9 nuclease. TALE-based approach outperforms the Zinc-Finger based tool by exhibiting better DNA recognition, reduced off target effects and easier construction. See Pereira C V et al., EMBO Mol Med. (2018):e8084; Bacman S R et al., Nat Med. 2013; 19(9):1111-1113; Doyle E L et al. “TAL Effector-Nucleotide Targeter (TALE-NT) 2.0: tools for TAL effector design and target prediction”. Nucleic Acids Res. 2012; 40 (Web Server issue): W117-W122. The CRISPR-Cas9 method has garnered much success in the recent years, however, it has not been used as successfully for the mitochondrial genome whereas

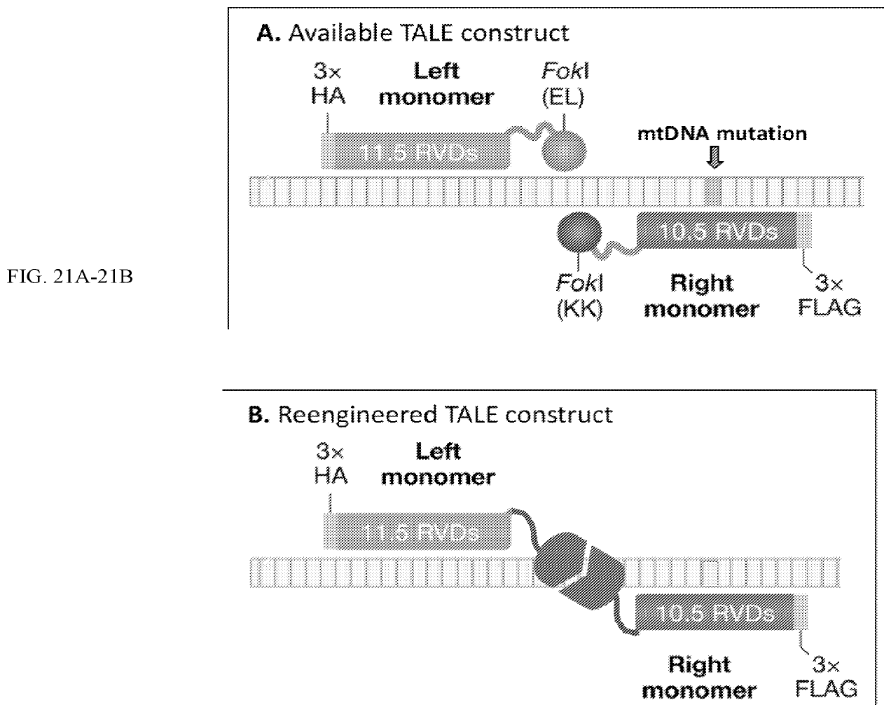

TALE-derived constructs have successfully been used to target the mitochondrial genome. Thus, they are the ideal tool for the development of mtDNA sequence specific reporters. See FIG. 21. TALEs are derived from the Xanthomonas bacterium, in which they are used to bind and activate promoters. The binding specificity of the DNA binding domain of TALEs comes from a modular build of repeats, each of which contain 33-34 amino acids (aa) with the two amino acids at position 12 and 13, the repeat variable diresidue (RVD), conferring specific DNA base recognition (FIG. 21). The length of the DNA recognition site that allows for specificity and reduces off-target binding depends on the size and complexity of the genome. Recognition sequence between 8-12 base pairs have been sufficient for targeting mammalian mtDNA genomes. Two sets of such TALE constructs can be designed where one pair recognizes the WT mtDNA sequence, deleted in the uaDf5 mutant mtDNA, and a second pair of TALE constructs which recognize sequences flanking the deletion (FIG. 19A-19C). The TALE domains of each construct will be developed following published methods^{10,11}. The TAL Effector-Nucleotide Targeter (TALE-NT) 2.0 website will help design the TALE components of the reporter construct, and the molecular cloning tool Golden Gate TALEN with the TAL effector kit 2.0 will help generate the DNA vector containing the constructs. Each vector construct will contain the TALE module with a triple HA (hemagglutinin) tag driven by a common mammalian promoter built into the Golden Gate TALEN and TALE effector kit. The final DNA vectors will be sequence-verified and the function of the protein product will be tested in vitro.

HEK 293T cells will be transfected with the newly synthesized DNA vectors to enable TALE expression and subsequent purification. HEK 293T cells will be used since they have high transfection frequency and creating transgenic worms for the purposes of verification alone is prohibitive. Once the TALE modules are purified using the HA tag, each TALE module will be tested by incubating it with either (a) DNA oligomers matching the sequence the TALEs were designed to recognize or (b) random DNA oligomers that the TALE motif should not bind. These samples will then be run in separate lanes on a gel and DNA will be visualized to observe whether the samples run at a high molecular weight, which would indicate binding to the TALE, or at a low molecular weight, which represents unbound DNA. Off target binding is not as much of a concern since the reporter construct is split; however, it is possible that the TALE construct exhibits insufficient binding to its corresponding DNA sequence. Due to this possibility, three different TALE pairs will be designed simultaneously for both WT and mutant mtDNA, but they will be tested sequentially in order to minimize effort in the event that the first TALE pair works efficiently.

Self-complementing split fluorescent proteins (FPs) are split FP constructs in which the two fragments can associate by themselves to form a fully functional FP without any assistance aside from being in proximity to each other. This tool is ideal for the proposed reporter construct to limit background fluorescence. The split FP will remain dark unless brought together by the TALE motif bound to its recognition sequence on the mtDNA (FIG. 19A-19C). While several different split FP constructs are available, their capacity to reconstitute and function in the mitochondrial matrix remains untested. Considering that various FPs have been successfully targeted to the mitochondrial matrix in various different organisms, split FPs should function in the mitochondrial matrix.

The mNeonGreen2_{1-10/11} (mNG2) and red fluorescing sfCherry2_{1-10/11} split FPs have been recently evolved to produce higher fluorescent signal¹⁵ and will be assessed for reconstitution in the mitochondrial matrix. The oxidative nature of the mitochondrial matrix is conserved across species and thus, the split FPs can be tested in HEK 293T cells and be applied to any organism. The advantages of using cells for testing purposes are that (a) HEK 293T cells are easily transfected and (b) there exists a paired TALE construct designed to recognize and digest mutant human mtDNA. The existing paired TALE construct will be redesigned to (a) recognize human WT mtDNA by changing the RVD code for one of the repeats one of the TALE monomers (FIG. 21A), and (b) carry the split FP domains instead of the nuclease domains (FIG. 21B). These changes will be accomplished by reengineering the existing DNA vector carrying the original TALE constructs. After creation and sequence verification the resulting DNA vectors can be transfected into HEK 293T cells to be imaged by confocal microscopy to confirm the ability of the split constructs to reconstitute and fluoresce in the mitochondrial matrix.

Construction of C. elegans Reporter Strain(s) to Visualize Wildtype and Mutant mtDNA.

The final mtDNA sequence specific fluorescent reporters will be created in the uaDf5 C. elegans strain carrying a mixed population of WT and mutant mtDNA. Two DNA vectors will be generated; one will harbor the construct that recognizes the WT mtDNA and the second one will carry the construct that recognizes the mutant mtDNA. Each DNA vector will contain both components of the reporter construct, and each component will be built by adding a mitochondrial localization sequence, a TALE module and one part of the split FP (FIG. 18). Both components will be driven by a C. elegans gene promoter that is active at a moderate level in all tissues. Two different colored split FPs will be used to generate the WT and the mutant reporter constructs. Once these constructs are created and sequence-verified, they will be delivered to Nemametrix, a biotechnology company that provides fee-for-service services for generating transgenic worm strains. Once the two strains created in the uaDf5 are made, these reporter strains will be mated to produce one strain, which will be observed using microscopy. First, we will determine that there is fluorescence as expected, in punctate expression in the mitochondrial matrix. To ensure that the localization is correct, all DNA in the worms will be stained with Hoescht stain and the mitochondria will be visualized by using a MitoTracker dye. As a final test of the reporter tool, mitophagy, a process of mitochondrial degradation known to affect the relative frequencies of the mutant mtDNA, will be modulated and the relative frequency of mutant mtDNA puncta to the WT mtDNA puncta will be observed. The observations will then be correlated to the published data to make certain the reporters are behaving as expected. The beauty of the design proposed here is that most of these individual modules have been well studied in different contexts and it is anticipated to be straightforward to develop the reporter constructs.

### Discussion

Tens to hundreds of mtDNA molecules reside in relatively hostile environs inside the mitochondrial matrix and healthy cells still maintain enough mtDNA genomes that are free of pathogenic mutations. This balance can be disrupted as cells age and accumulate mutations, high mtDNA mutation load is inherited, or exposure to toxins increase mtDNA mutations. When the level of mutated mtDNA genomes becomes pathogenic, it leads to a variety of mitochondrial diseases that have mild to severe neuromuscular and metabolic dysfunction. In order to tackle the mutated mtDNA genomes in the context of disease, understanding the mechanisms by which healthy cells clear mutated mtDNA can identify new targets that can be exploited for developing therapies. While it is possible to observe mtDNA genome in cells and organisms, visualizing the mtDNA genomes in a sequence-specific manner has not reported to be possible yet. This is an important tool since the mutant and WT mtDNA genomes exist in a mixed population and when biological pathways are modulated to reduce the frequency of mutated mtDNA, the direct consequence of modulating biological pathways on the mtDNA genome populations remains unknown. Uncovering the direct consequences will allow insights into the fundamental mechanisms that maintain mtDNA fidelity.

The tool described herein allows for real-time, in vivo observation of mutant versus WT mtDNA genome dynamics in different tissue types simultaneously, thereby breaking a major barrier in the field of mutant mtDNA clearance. For example, it is possible that when high frequency of mutant mtDNA genomes stochastically exist in proximity and produce faulty respiratory complex subunits, it triggers a local disruption of the mitochondrial membrane potential. This disruption could signal for mitochondrial fission and ultimately lead to the degradation of the small, newly-formed mitochondrion that has a high frequency of mutated mtDNA genomes, and thus will preferentially eliminates the mutated mitochondrial genomes. Such a string of events is a possibility; however, it cannot be properly addressed without a visualization tool that distinctly recognizes mutant versus WT mtDNA genomes in real-time under different contextual states. The tool described herein enables observation of WT vs SLSMD mtDNA genomes in a new way to address previously unanswerable questions and open up new avenues of query into understanding mtDNA maintenance.

### REFERENCES FOR EXAMPLE III

Pereira C V, Bacman S R, Arguello T, et al. mitoTev-TALE: a monomeric DNA editing enzyme to reduce mutant mitochondrial DNA levels. EMBO Mol Med. 2018; 10(9):e8084. doi:10.15252/emmm.201708084
Bacman S R, Williams S L, Pinto M, Peralta S, Moraes C T. Specific elimination of mutant mitochondrial genomes in patient-derived cells by mitoTALENs. Nat Med. 2013; 19(9):1111-1113. doi:10.1038/nm.3261
Nissanka N, Moraes C T. Mitochondrial DNA heteroplasmy in disease and targeted nuclease-based therapeutic approaches. EMBO Rep. 2020; 21(3):e49612. doi:10.15252/embr.201949612
Thakore P I, Gersbach C A. Design, Assembly, and Characterization of TALE-Based Transcriptional Activators and Repressors. Methods Mol Biol. 2016; 1338:71-88. doi:10.1007/978-1-4939-2932-0_7
Doyle E L, Booher N J, Standage D S, et al. TAL Effector-Nucleotide Targeter (TALE-NT) 2.0: tools for TAL effector design and target prediction. Nucleic Acids Res. 2012; 40 (Web Server issue): W117-W122. doi:10.1093/nar/gks608
Kaddoum, L., Magdeleine, E., Waldo, G. S., Joly, E. & Cabantous, S. One-step split GFP staining for sensitive protein detection and localization in mammalian cells. Biotechniques 49, 727-736 (2010).
Van Engelenburg, S. B. & Palmer, A. E. Imaging type-III secretion reveals dynamics and spatial segregation of Salmonella effectors. Nat. Methods 7, 325-330 (2010).
Kamiyama, D. et al. Versatile protein tagging in cells with split fluorescent protein. Nat. Commun. 7, 11046 (2016).
Feng, S., Sekine, S., Pessino, V. et al. Improved split fluorescent proteins for endogenous protein labeling. Nat Commun 8, 370 (2017). https://doi.org/10.1038/s41467-017-00494-8

Sequences useful in the practice of the invention include, without limitation:

```
● Autophagy-related protein 9 (atg-9) [C. elegans] GenBank ID: 178561
          o Available on the world wide web at
                    ▪ ncbi.nlm.nih.gov/gene/178561
                    ▪ ncbi.nlm.nih.gov/nuccore/NM_070777.5
                    ▪ ncbi.nlm.nih.gov/nuccorc/NM_001377584.1
    ● Protein Sequence - SEQ ID NO: 29
MFNSQSKRAYQQIDDDFDDEVLRNSTCTSRFMQGWGSSTRSLLFGGASNDEQRNLIASS
SSHHSYHDSPAEEPPETHYEQFTATHNHGPPTMASSSQLNSRRWDHVLNLDEFFTHIYE
YHQNGGYLCIVLQKVFSLLQFIFVMSFTTFFTQCVNYQFLFANTNVTSHGTVNQGKRHF
GDAVVDNCPAHISIWMIFAILAAIVYWITRVIKHAQYIMKMSEIQQFYAHELKIADDQLP
NLTWHAIVKRICEAQKKLRLSIHQDNITSIYIYHRILRYKNYMTGMINKRILHPVFDVPFL
GPIAYLPNNLKHEIERILFTSSTSAWTNGPNLREEYKHHEQLDMAAKKMKEDCKFYGFL
SLVLMPLLLPFQIMESFFSLTELIKRRPDGLGMRRYSNFGRYLLRHFNELDHELSARLNR
SHIYAAAYMDQFFSPVLEIAAKNITFIAAAVFGVLTILSAWDEDVLQVEHVITVLTICGIV
VLVCRGMIPDENLVWQPEILMTHVTSELHYLPSTWKGKAHTTGVRHEFDQLFQMKWM
FFVLELTSPIFTPFVLLFWLRPRCSQLANFFHDYTERVDGLGDVCSFAVMDVGKHGDPK
WNHIKELKAIVEDQEDQQQAQSVVTSLNRARDGKTELSILHFKTTNPEWQPPKASEKFL
RKFRNRLGQEASMLAPLTSMHLGQQMDRQQQQDQILPPRNILLESVHSIVPTTSGGISAS
QVAPGRHPLIGDGLHRIDGPVGNAFQGIQGAKLGSGGVLASLYQEQPRAAESLSNSLRA
SGVDIDGAGAEMRINALFLRGLHDESIIHSSSRNYGGTTSSFNMHPTAMQSVFAMPDGF
GQPAPAVESSLIDIETPTYHRESVQRSAAEHKMEHETPEPLAELPDLPGPSSSEHQQRSRL
VPNTQHRQINEEEEEEEEEDNTPPLSFSS
        ● DNA sequence - SEQ ID NO: 30
atgttcaact cacagtcaaa acgggcatat cagcaaatcg acgacgattt tgatgatgaa gtgctgcgta acagtacgtg
tacgtcacga tttatgcaag gatggggctc atccacaagg tcactgcttt ttggaggcgc ttccaatgat gaacagagga
atttaatcgc cagctcctcg tcccatcact cgtatcacga ttcacctgcc gaggagccac cggaaactca ctacgaacaa
ttcacggcca cccacaatca tggtccgcca acaatggctt catcgtcaca gctaaactct cgacgatggg accacgtact
gaatttagac gaatttttca cgcatatcta cgaatatcat cagaatggcg gttatttgtg cattgttttg cagaaagtgt tcagtttatt
acaatttata tttgttatga gttttactac attcttcact caatgcgtca actatcaatt cctattcgcc aacacaaacg tgacaagtca
cggcactgta aatcagggaa aacggcattt cggagacgctgtcgtcgata attgcccggc acacattagt atttggatga
ttttcgcgat tctcgccgcc atcgtctact ggatcactcg ggtcatcaaa cacgctcagt acattatgaa aatgtcggaa attcagcaat
tttatgctca cgagctgaaa attgccgacg atcagctgcc caatttgacg tggcacgcga ttgtgaagag gatttgtgag
gcgcagaaaa aactacggtt gagcattcat caggataata ttacgtcgat ttatatttat catcgcattc ttcgttataa gaattatatg
actggaatga ttaataagcg aatccttcac ccagtcttcg atgttccttt cctcggacca atcgcctatc ttcccaacaa tctaaaacac
gaaatcgagc gaattctgtt cacaagttcc acgtcagcat ggacgaatgg tccgaacctc cgagaggagt acaaacatca
cgagcaactc gatatggcag ccaagaaaat gaaggaagat tgcaaattct acggatttct gtcgctcgtc ctgatgcctc
tactgctccc attccagatt atggaatcgt tcttctcact gacggagctc ataaaaagac gaccagatgg gcttggaatg
agaagataca gtaattttgg aagatacctc ctgcgtcact tcaacgagct tgaccacgaa ctctcggctc gcctgaatcg
ttcacacatt tacgcggcag cctacatgga tcaattcttc tccccagtcc tggaaatcgc cgccaaaaat atcacattca
tcgccgccgc cgtcttcgga gtgctgacaa ttctgtctgc gtgggatgag gatgttctcc aggtggaaca cgtcatcacc
gttctcacga tctgcggaat cgtggttctc gtgtgtcgtg gaatgattcc cgacgagaat ctcgtctggc aacccgagat
cctcatgacc cacgtcactt cggagcttca ctacctgccg tccacgtgga agggaaaggc acatacgaca ggcgtcaggc
acgaattcga tcagcttttc cagatgaaat ggatgttttt tgtgctggaa ctgacgtcgc cgatctttac accattcgtt ctgctgttct
ggttgagacc caggtgctca cagctcgcga atttcttcca tgactatacg gaacgagtcg atgggctcgg cgatgtgtg
agcttcgctg tgatggatgt tggaaagcat ggtgatccga agtggaatca tattaaagag ctcaaagcga tcgttgagga
tcaggaggat cagcagcagg cccagtcggt ggtgacatca ttgaatcggg ctcgcgatgg aaaaacggag ctatcgattc
tccatttcaa gactaccaat cccgaatggc agccgcctaa ggcgtcggag aaatttttga ggaaatttag gaatcggttg
gggcaggagg cttccatgct cgcaccgctg acctcaatgc atctcgggca gcaaatggat cggcagcagc agcaggatca
aatcctgcca ccgcgcaaca ttctcctcga atccgtacac tcgattgtcc caacaacttc cggcggaatt tcagcttccc
aggtcgcacc gggccgccat ccactcatcg gagacggcct tcaccgtatc gacgggcccg ttggaaacgc gttccaggga
attcagggag ccaagctggg cagcggtggt gtgctcgcca gcctctacca ggagcagcca cgtgccgccg agtcgctgag
caacagtttg agagctagtg gagttgatat cgatggagcc ggagccgaaa tgcgcatcaa cgccttgttc ctgagaggtc
Itcacgatga gagtattatc cacagctcat cccgaaacta cggcggcacg acgtcaagct tcaacatgca cccgaccgct
atgcaaagcg ttttcgcgat gcccgacggc tttggccagc cggctcccgc cgtagaatca tcccttatcg acattgaaac
acccacctat cacagggaat cagtgcagag atcggcggcg gagcacaaga tggagcacga gactcccgag ccactggccg
agcttccgga tcttccggga ccgtcatcgt cggagcatca gcagagatcc cgactcgttc cgaacaccca gcatcggcag
attaatgagg aggaagaaga ggaagaggag gaggacaata cgccaccgct tagctttagc tcttagattt tctgaattaa
aaaaaaaaaa tttttttaat ccctgcatta tttcctcgtt ttcccctacc aattgacgtg aaaacgcgct ccgcctccaa accatgggtc
tcgttaggta tttggcgcga aaaccgacaa tttgaacgta ttcaccaatt ttcaagcatt taaaattatt tttaagtcga atttcggcaa
aaacacgaaa aaccgttttt ttttttcgat tttcaagcat tttttgtatt tttaaaaatg atttttacgt tgatttttcg cataaaaacc
gaaaacatct gaaaattaaa aaaaatcaca aaaaaaaaat taaataattt tttccgattt caacaatttt caagcatttt ttgtctggtt
tttatgccat tccggttaaa aaaaaaaact ctaaaatacg aaaaatgctt gaaaaagtga caaaaacgat taaaacaagc ttttttattg
atttttgcgt cgaatttcgg cttaattatc cataaaaacc gggaaaagct cgaaaatttt ttaccgtttg cgggggtggg acgttttcaa
tttgccgtgg agcgcgttta cacggttttc acccaatttc taacctgaac tgcagcataa ttttgtacat ttttgtgtat gttgtaccgt
tttctgttgt ttttttttgt aaattttgaa actttgacac gagtttacat tagtcagaat agtttttttt tctatgtatg aacacaattt
tttttcgaat tttttttcac cgcagctccc tcaccgctac tctgtacaat ttttttcact gacaatctga ataaaatccc gcaaatttg
      ● NIP3 homolog (dct-1) [C. elegans] GenBank ID: 181053
                o Available on the world wide web at
                        ▪ ncbi.nlm.nih.gov/gene/181053
                        ▪ ncbi.nlm.nih.gov/nuccore/NM_076938.7
                        ▪ ncbi.nlm.nih.gov/nuccore/NM_076939.5
                o Protein Sequence - SEQ ID NO: 31
MSSFLEFAKPKMLDIKRKINFASGEKTDESVQPQQQTEQSSAQQTTPSAKAVSNPFITPLT
ESTPGMSESWVELAPSRTSLCSSVDINMVIIDEKDKDSRLSPVSIAQSPHVEFESLEQVKY
KLVREMLPPGKNTDWIWDWSSRPENTPPKTVRMVQYGSNLTTPPNSPEPELYQYLPCES
DSLFNVRVVFGFLVTNIFSFVVGAAVGFAVCRKLIKHHRQ
                o Nucleotide Sequence - SEQ ID NO: 32
agacgggtct ttcctaacaa tgtcctcatt tcttgagttt gcaaaaccca aaatgctcga tatcaaaaga aagatcaact ttgcgtccgg
cgagaagacg gatgagtctg tgcaaccgca acagcagacg gagcagtcat cggctcaaca gacaacacca tccgcaaaag
ccgtctcaaa cccatttatc accccattga ccgagagtac acctggtatg tcagaatcgt gggtggaact ggcaccaagc
cgtaccagtt tgtgcagtag cgtcgatatt aatatggtga tcattgatga aaaagataag gattcgaggc tgagcccagt
ctcaatcgca caatctcctc acgtggaatt cgagagctta gaacaagtaa agtacaagtt ggttagagaa atgcttcctc
ccggaaagaa tactgattgg atttgggatt ggagcagtcg gccagaaaat acacctccaa agactgtccg tatggttcaa
tatggctcca accttaccac tccgccaaac tctcccgagc cagagctcta ccaatacttg ccatgcgaat ctgattcgct tttcaacgtt
cgcgtagtct ttggtttctt agtcaccaac atcttcagct ttgtcgttgg agcagcagta ggatttgctg tttgccggaa actaatcaaa
catcatcgtc aataattcca aacaaaaatt ttaacgttcc acttacctca tttcactgta catacattcc agcagtgctg gagattgggc
atttttaccc tttgattcac aatactacat tctctgcaaa attcttccgg agicttcttg calcttgtca aaaagattcg aataaacaat
aattttgcca tgtttccctt ctctccaaat tgtcatcttt caaactgtct cctgaatatt cctaatactc aaaacattcc tactacagtt
ttgaattcaa cacatctaat cgcgatttgc tctcacgatt aggtaatttt aaattaatta tgtacataat atagctttat acactgaatg
taattacgac tttttctttg ctgttacctg ggcattttga ttctgaaact tgcttttcca tgtttttctg tcatcacaaa ttgtaaatat
ttctaaaata ctgttacaaa accacgcatt ttaataaaat gtaaatat
      ● FUN14 domain-containing protein (fndc-1) [C. elegans] GenBank ID: 174714
              o Available on the world wide web at
                        ▪ ncbi.nlm.nih.gov/gene/?term-Caenorhabditis+elegans+fndc-1
                        ▪ ncbi.nlm.nih.gov/nuccore/NM_064003.7
              o Protein Sequence - SEQ ID NO: 33
MVDLSKNDGGSGKAGKGVSDAIDTVLYYVVDLKKQQPMVQLGVGAGFGTVTGYFVT
KGGRLVAATVGISELLAQFAIHKGYITLNESKIERDMKNLHKSVMNKVSGKKVINISDSF
VSEYRWILGGFAAGMLIGESVA
              o Nucleotide Sequence - SEQ ID NO: 34
ccagccatgg ttgatctttc aaaaaacgac ggaggcagtg gaaaagccgg aaaaggtgtg agtgatgcca tcgatacagt
cctctactac gttgttgatc tgaaaaagca gcagccgatg gtccagctgg gtgttggtgc tggattcggg acagtgactg
gatattttgt caccaaggga ggacgtcttg ttgctgccac agtgggaatc tctttcctgt tggctcaatt cgctattcac aaaggataca
tcacgctgaa cgaatcaaaa atcgaacgag acatgaaaaa tctacacaag tccgtcatga acaaagtatc cggcaagaaa
gttatcaaca tttccgattc atttgtctcc gaataccggt ggattcttgg cggattcgca gctgggatgc tgattggatt tagcgtcgcc
taaatgtgga aaattgtgaa gcttcagttg gtttttatct acgatctgta ctatttataa tcgagttgct cgatatgatg cataaataac
tttttctcac tctccattgt ctagtcatgc tgtctatttt tttcttcaca ttgactgata aattatacac cctt
      ● E3 ubiquitin-protein ligase hrd-like protein 1 (hrdl-1) [C. elegans] GenBank ID: 172833
                o Available on the world wide web at
                          ▪ ncbi.nlm.nih.gov/gene/172833
                          ▪ ncbi.nlm.nih.gov/nuccore/NM_060201.6
                o Protein Sequence - SEQ ID NO: 35
MNTGNPDTRRNNQVPMGVVNLLSRSSFPSVDSYLALSVLVAIVASVTVFTTFRSQPELQ
KLIEEELRNNTRLSSAYGLNIEALSGHTFFQIAHYILSDTTLIWVAINSYFAILAVCTRLIIK
LTFKELARQEENVARQAFFCYVLLTIVYLSVVIGPQKGHRVMPWMIWGGICAFLSHLQF
ITCQRLKHISPSCDRGSQKISFLSLFLFFVSIAMTFLISRFQHHLTWQPAVLLYFDCLLAVF
RSTYILFRCISSSRVFSFNPDSVRHFNYWLELITNFVCELIQMLSFAQLLAFSPGLNLTSIFF
LYHMKLTYNCMTEQLSRHRNHKKIFEHIERSYPSVKCANGDDRCVVCWELLGTSRRLP
CSHQFHDWCLMWWLAQDSSCPTCRCTIPSPQDQIRQPPEVGNSTRLRFNGGSFGFVHFP
AFTLEVAANFGPFFGRAAEPTEEQLQTMLEQVREMFPQMSVDIIMTDLRQSGSAQSTIEN
ILEGRIGMNASFMPGGVLDDELSDESENEIEYEEPAEIVQEPDNGRQRTWTKLSSSSGDE
DLSYYEIQRAKMIETYRRKYLESDKAADLRAMGITE
                o Nucleotide Sequence - SEQ ID NO: 36
atgaatacgg ggaacccgga tacccgtcgt aacaatcagg tacccatggg ggttgtcaatctactatccc gatcatcatt tccatctgtc
gactcatatc tggcattgtc agttcttgtt gcaattgttg cttctgtcac tgtatttaca acatttcgct cacaacccga gctacagaag
cttatcgaag aagaacttcg gaataataca cgactgtcat cagcatatgg tttgaatatt gaagcattgt ccgggcacac attctttcaa
attgctcatt acattttatc tgatacaacg ctaatctggg ttgctataaa ctcgtatttt gccatactag cagtttgtac gagactaata
atcaaattaa cattcaaaga gctcgcccga caggaggaaa atgtggcccg tcaagcttt ttctgttatg tcctgttaac aatcgtctac
ttatcagttg tgatcggacc gcaaaagggg catcgtgtaa tgccatggat gatctggggt ggtatttgtg ctttcttatc ccatttgcag
tttatcacat gtcagaggct caagcatatc tctccatcct gtgatcgtgg aagtcagaag atttcattcc tctcgttgtt cctcttcttc
gtttcgattg ccatgacgtt cttgatttct cgattccaac atcacttaac atggcaacct gctgtacttt tatattttga ttgccttctc
gcagtattcc gttccactta cattctgttc cgatgcattt cttcatctcg tgtattctcc ttcaatcctg actctgttcg tcatttcaac
tattggctcg agcttatcac caactttgtc tgcgaactca ttcaaatgct cagttttgct caacttttgg cattctcacc tggacttaat
ctcacaagca tcttcttctt gtatcacatg aagcttacct acaactgcat gactgaacaa ttaagtcgtc atcgtaatca taagaagatc
ttcgaacaca ttgagagatc atatccaagt gttaaatgtg ctaatggtga tgatcgttgc gttgtttgct gggaattgct cggaacatct
cgaagacttc catgcicica tcaattccat gattggigic tcatgiggig gcttgctcag gattcttcat gtccaacgig tcgttgtact
attccatctc cacaagatca aatcagacaa ccaccagagg ttggaaatag tacaagatta cgtttcaatg gtggatcatt tggattcgtt
catttcccag cattcactct tgaagtagct gctaacttcg ggcctttctt tggtcgagca gccgaaccaa cagaagagca
attgcaaaca atgctcgagc aagtcaggga gatgttcccg cagatgtctg ttgatataat aatgacggat ctacgacaat
cgggatccgc tcagtctaca atcgaaaata ttctagaagg aaggatagga atgaatgcat catttatgcc cggtggggtt
ttggatgatg aactctcaga tgagagtgaa aacgaaatag aatatgaaga gcccgccgaa atagtccaag aaccagataa
tggccgtcaa agaacgtgga caaaattgag ttcttcatca ggagatgaag atctatcata ctatgaaatc cagcgagcca
aaatgatcga aacttaccga cggaaatatc tggagtctga taaagctgcc gatctacgag caatgggaat caccgaataa
ttcatactca tttctctcct cttttctgtt tacttaggat gttcttgtca tttcaaagct acctggtagc gtactctcat tatttctaaa
ttcatgcctg tatgagtata gttttttgtt gtatattttg tatccatttg atgagattct ctgagattcc catctttcat catatttagc
atagttccac ccttttttc ctttcctttc atgaccctct tttcatgtat gggtatgatc atcatccaca ccaactatca attttctctc
tgtatcctct atcgagattc tgaatctcgt ttcttgttc cttttttccg tgtttcctct gaaatctcga ctatcattct aatcctttac
aattctcatc ccggtacctt gtgagtgtga acctgtacat tttttctccg atccccattt ttcaccaaag taaatacttt gagttttc
      ● Mucin Like Gene (mul-1) [C. elegans] GenBank ID: 186063
                o Available on the world wide web at
                          ▪ ncbi.nlm.nih.gov/gene/186063
                          ▪ ncbi.nlm.nih.gov/nuccore/NM_068086.6
                          ▪ ncbi.nlm.nih.gov/nuccore/NM_001313027.3
                o Protein Sequence - SEQ ID NO: 37
MHYYILPILFSIYIIATCNLCEDSMITCPDLKDQCDQGFVQSQCPYTCGICHQDPNQCTDT
NLDCLKFDSMCKTDVKYQKQCPYTCGTCVPATPDSCFDHMIECPNYSVPCTDAVKIQCP
KRCGVCTSGASNSTLAPPRPSPTPPCFDSGNECATYTLPCDVNQKIFCPRTCGVCGSTGV
PMAQTTLLTTVKPTTTVVPSTTTKLPATTTKKLPSTTPKPPCKDSSPNCAGWAKNGFCT
NTFYPPEKRKEYCAKTCRMC
                o Nucleotide Sequence - SEQ ID NO: 38
acttttaaac aaatatcatc attattttaa tcaccatcat catcatcttg gtttctaacc cccatcttaa ctgataacac actgaaactt
ttgttataaa acaaggcatt acatcaagta ttatgtacta gattttccca caaaaaatgc actactacat cctgcctatt cttttttcaa
tatacattat tgcaacttgc aatctatgtg aagactcaat gatcacatgt ccggatctca aggatcaatg tgatcaggga tttgtgcaga
gtcaatgccc atacacttgt ggaatatgcc atcaggatcc aaatcaatgt actgacacta acttagattg cttgaagttt gattcaatgt
gcaagacgga tgtgaagtat cagaaacaat gcccatacac ctgtggaacc tgcgtaccag ctaccccgga cagctgcttt
gatcatatga ttgagtgccc aaattatagc gttccatgta ctgacgctgt taagatacaa tgcccaaaaa ggtgtggagt ttgcacctcg
ggagccagta attcgacatt agctcctccc agaccgtcac caactccacc atgttttgat agtggcaacg aatgcgccac
ctatacgctg ccatgtgatg tgaaccaaaa gatattctgc ccacggacct gtggagtttg tggaagtact ggggtaccta
tggctcaaac tacgttgctc actacggtaa agcctacgac tacagtagta ccaagtacta caacaaaact gccagctacc
acgaccaaga agcttccatc aactacgcca aagccaccat gaaggattc gtcgccaaac tgtgccgggt gggcaaaaaa
cggcttcigc acgaacacct tctatccacc ggagaagaga aaggagtact gigcaaagac atgtagaatg lgcigatttt ttgatttaat
aaattattta ttctt
      ● E3 ubiquitin-protein ligase parkin; RBR-type E3 ubiquitin transferase; Ubiquitin-like
          domain-containing protein (pdr-1) [C. elegans] GenBank ID: 176816
                o Available on the world wide web at
                          ▪ ncbi.nlm.nih.gov/gene/176816
                          ▪ ncbi.nlm.nih.gov/nuccore/NM_001306674.3
                          ▪ ncbi.nlm.nih.gov/nuccore/NM_001372678.3
                          ▪ ncbi.nlm.nih.gov/nuccore/NM_001383028.2
                o Protein Sequence - SEQ ID NO: 39
MSDEISILIQDRKTGQRRNLTLNINITGNIEDLTKDVEKLTEIPSDELEVVFCGKKLSKSTI
MRDLSLTPATQIMLLRPKFNSHNENGATTAKITTDSSILGSFYVWCKNCDDVKRGKLRV
YCQKCSSTSVLVKSEPQNWSDVLKSKRIPAVCEECCTPGLFAEFKFKCLACNDPAAALT
HVRGNWQMTECCVCDGKEKVIFDLGCNHITCQFCFRDYLLSQLERFGFVNQPPHGFTIF
CPYPGCNRVVQDVHHFHIMGQTSYSEYQRKATERLIAVDDKGVTCPNVSCGQSFFWEP
YDDDGRSQCPDCFFSFCRKCFERNCVCQSEDDLTRTTIDATTRRCPKCHVATERNGGCA
HIHCTSCGMDWCFKCKTEWKEECQWDHWFN
                o Nucleotide Sequence - SEQ ID NO: 40
ctctcatgaa gcatgtctga tgaaatctct atattaatac aagatagaaa aacaggtcaa cgtaggaatc taacacttaa tataaatata
actggaaata tcgaagatct cacaaaagat gtggaaaagc tcaccgaaat tcccagcgat gagctggaag tggttttctg
tgggaaaaag ttatcaaaat caacgattat gagggatttg tcactgacac ctgcaacaca aatcatgctt ctccgtccaa
agttcaatag tcacaacgaa aacggtgcta ctactgcaaa aataacaaca gattcttcaa ttctcggaag cttctacgtg
tggtgcaaaa attgtgacga cgtcaagcgc ggcaaactgc gggtttattg ccaaaaatgc tcgtcaacct ctgttctagt
caaatctgaa ccccagaact ggtccgacgt tctcaaaagc aagagaatac cggcggtctg cgaagaatgc tgtactccag
gictuttcgc igaattcaag ttcaaalgic tagcctgcaa cgatccggcc gcagctctaa ctcacglacg cggaaattgg
caaatgaccg agtgctgtgt ttgtgatggg aaggagaaag tgatcttcga cctcggatgc aatcatatta catgccaatt ctgtttcaga
gattatttgc taagtcaact ggaacgattc ggttttgtca atcagccgcc gcatggcttc accattttct gcccctatcc agggtgcaat
agagtggtac aagatgtgca ccatttccac attatgggtc agacgtcgta cagcgaatac caacggaaag ccaccgagcg
attgattgcc gtggacgaca agggtgtgac ttgcccgaat gtctcgtgtg ggcagagctt cttctgggag ccctatgatg
acgatggaag atcccagtgt ccagattgtt ttttttcgtt ttgcagaaag tgcttcgaaa gaaattgtgt gtgccagagc gaagacgatc
tcacccgaac tacaattgac gcgactacaa gaagatgccc aaaatgccac gtggcaaccg aacggaacgg cggatgtgct
cacattcact gtacctcgtg tggaatggat tggtgtttca agtgcaagac agaatggaag gaagagtgtc aatgggacca
ttggtttaat taataactct ctaaaattca tcttttctag agattgtacg tgttattctg tagattttta caacaattac ggtgtgtctg
aagttacatt gacatt
      ● Scrinc/threonine-protein kinasc pink-1, mitochondrial (pink-1) [C. elegans] GenBank ID:
          173918
                o Available on the world wide web at
                          ▪ ncbi.nlm.nih.gov/gene/173918
                          ▪ ncbi.nlm.nih.gov/nuccore/NM_062616.7
                o Protein Sequence - SEQ ID NO: 41
MSMKRFGKAAYRIANELVAKGGRLPIFQRFLPRIFPATYNLGVHVVLKKAPFPRQNALR
IARLVTRHGRVFRPFSSVIIERHRFQNQNDWRRKFQPIRKELPRNVDLVERIRQIFGNSLR
YNEDLKSTEWPNRIDSYEFGEFLGQGCNAAVYSARLANSDAESSGNTHYGAGFNEVTNI
LAEIPPVSKVAQKKFPLAIKLMFNFEHDRDGDAHLLKSMGNELAPYPNAAKLLNGQMG
TFRPLPAKHPNVVRIQTAFIDSLKVLPDAIERYPDALHTARWYESIASEPKTMYVVMRRY
RQTLHEYVWTRHRNYWTGRVIIAQLLEACTYLHKHKVAQRDMKSDNILLEYDFDDEIP
QLVVADFGCALACDNWQVDYESDEVSLGGNAKTKAPEIATAVPGKNVKVNFEMADT
WAAGGLSYEVLTRSNPFYKLLDTATYQESELPALPSRVNFVARDVIFDLLKRDPNERVK
PNIAANALNLSLFRMGEDVKQMMEKCGISQMTTLLAGSSKVLSQKINSRLDKVMNLITA
ETIMANLAPHLISRAERQLRATFLSRMNREDIWRSLQYFFPAGVQLDTPATSSDCLETISS
LMSSFSNDSENYEKQQKPAKNGYNNVPLLLRNVIRTDADGINGIVHRVRSK
                o Nucleotide Sequence - SEQ ID NO: 42
Iglatagaag taagaatgtc tatgaaacga ttcggaaaag cagcatalcg aalcgcaaat gagttagttg caaaagglgg
acgactacca attttccaac gcttcctgcc gagaatattt cccgccactt ataatttagg agttcatgtc gtactcaaaa aggctccatt
tccacgacaa aatgctctac gaattgctcg ccttgtaact cgccacggtc gagttttccg gccattttcc tcagtaataa tcgaaagaca
tcgatttcaa aatcaaaatg attggcgtcg aaagtttcaa ccgattcgta aagaattgcc aagaaatgtg gatttagtcg
aacgaatcag gcagatattt ggcaattctc tacgatacaa tgaggatttg aaaagcactg aatggccgaa tagaattgat tcttatgagt
ttggggaatt tctcggtcaa ggatgcaatg cagcagttta ctctgcgaga ttagccaatt ctgatgcaga atcctcaggg
aatactcact atggtgcagg gtttaatgaa gtcacaaata tacttgcaga aattccgcca gttagcaaag ttgcacaaaa
gaaattcccg ttggcaatca aattaatgtt taattttgaa catgatcgcg atggagatgc tcatctcttg aaatcaatgg gaaatgaatt
ggctccatat ccgaatgctg caaagttgct caatggacaa atgggaacat ttagacctct tccagcaaaa catccaaatg
ttgttcgaat tcagacagct tttattgatt cgttaaaagt tttgccagat gcgattgaac gatatccaga tgcccttcac actgcacgtt
ggtatgagtc aattgcctcc gaaccgaaaa caatgtacgt agtaatgaga cgataccgac aaacacttca tgaatatgta
tggactcgtc atcgaaatta ttggacagga cgagtgataa ttgctcaact attagaagca tgtacatatc ttcataagca taaagttgct
cagcgagaca tgaaaagtga taatattctt ctggaatatg attttgacga cgagattccc caattagttg tcgccgattt tggatgtgca
cttgcatgtg acaattggca agtagactat gaatcagatg aagttagtct tggaggaaat gccaagacaa aagcaccaga
aattgcgacg gctgttcctg gaaagaatgt aaaagtaaac ttcgaaatgg cagatacatg ggcagctgga ggcctttctt
atgaagttct aacacgatca aatccattct acaaacttct tgatactgca acataccagg aatcagaact accagcactc ccatctcgtg
tcaattttgt ggcacgagat gtcatttttg acctactcaa gcgagatcct aatgaaagag tcaagccgaa tattgctgca aatgcgttga
atttgtcatt gttcagaatg ggagaagatg tgaagcagat gatggaaaaa tgtggaatat ctcaaatgac tactctattg gctggaagtt
ctaaagtttt gagtcaaaaa atcaatagtc gtctggacaa agtgatgaat ctgattactg ctgaaactat catggccaac ctagctccac
atttgattag tcgagcagaa cgacaacttc gagcaacatt tctttcaaga atgaatcgag aagatatttg gagaagtctt caatatttct
tcccagctgg tgttcaactt gacacacctg ccacatcatc agactgtttg gagactattt ccagtttgat gtcgagtttt tcaaatgatt
cagaaaatta cgagaagcaa cagaaaccgg ctaaaaatgg atacaacaat gttccacttc ttctcagaaa tgttatccgt
acagatgcgg atggaatcaa tggaattgta catagagttc gatctaaata gatttgtttt aacgattact gtagtttgaa acctctagat
tacatatgta ataacctttt ttttttactt gttggtattt ttcaaattgt acaatcactt ttcatgccag tctattgcgt tttttgtct
aattacggtt tatcattccc tcatttatgg aaaatatcat acctt
      ● Plastin-1(plst-1) whole gene sequence (unspliced + UTR) - SEQ ID NO: 43
agactgctccgcgacggtgaaactcttgaagatctccggcgtctgtccccagaagaaattctcATGAGATGGGTTAATTAT
CATTTGGAACGTGCTGGAACTCAACGTCGCCTACACAATTTCACATCAGATATTGTC
GATTCAGAGATTTATACTCATTTGCTCCATCAAATTGCTCCAAATGGCTCCGGTGTCA
CGTTATCTCCACTCGGAGTCCATGGAAATGTCCCAAGAGCCGGAGCAATGTTAGATG
AAGCCGAGAAGCTTGATTGTCGGGAATTTGTGACAGCAACTGATGTGGCAGCCGGA
AATTACAAATTAAACTTGGCTTTTGTTGCCAATTTGTTCAATAAACATCCAAATTTGC
CGGATCCGGGAGCGGATGAAGTTGgtgagtttttgaaaaagtcgaaaattgactgaaaaataaaaaatttcaaaatttc
aaaatgacaacttccagcgattttaattaaaaaaggtccgataaaaaaaaaaaaalaagatttttcalcgaaaattcaccgactcaccgaagtc
gactagaattctgaaaatgacgatttttagccaaaaatttcaaattttttcatttttttggtgaaaaatttgaaatttttggctaaaaatcgtcattttca
gaattttaggcgactttcggtgaattttcgatgaaaaatcttattttttagactgataatcgccattttttgagaaaaatcggaaattttgaacaaaaa
tcattttctcattttcattttcagaattttaagcgaatttcagcgaaaaaatcggaccttttttaattaaaatcgctggaagttgtcattttcaaattttaa
gcgatttttggcgaaaaatttgaaatttctggccgaaaatcgtctttttcagaattttaggggcgacttttggcgaaaaatctaaaatatttaactga
aaaccactaaaacttgatcaattttaacctttttacttaaaatctctaaaaatcgattaattttcgattttttgatgaaaagttcgacattttttactgaaa
atcaattaaattttaaattttcagcgaaaaaatggagtttcttgtagaaaatcgttaaaaattgatcaatttttcaaatttttacactaaaaatcgtcatt
ttcaaaattacaggcaattttcgacaaaaaaataaagctaaaaattcgatttttaataaaaaatgaattttttttgctggaaatcgactttttcagattt
tcaaacgatttttgaagaaaaattcggagtttttgacttcaaatcgctaaaaattggccaattgtcgatatttaatgaaaaatctgaattttttttgctg
gaaatcgctcaaattctgaatttgcagcgaaaaaattgcattttttgttgaaaalcgctaaaaattgaataatttgcgtttttttttttggtgaaaaatc
ttaatttttgactgaaaattgtctggattcaactgtaaatttgtgaaaaactggaaaaatcgaaaaaattgatttttacatgaaaatcttgaatttttcc
gtaagttgtgtattgaaacacgaaaaatttcaatttttctccatattgcagctaaaaatctgaaaattttctaaaaatcaaattttatgaattttcagcc
aattttctggcaaaaattgaaatttttgagtaaaaaattacttcaaaaagatgaaaaattgacttctttttcgaaaaattcaatagaaatttgaattttt
aaaacacgaaaaataccaaaattccccacatttccgaacagtttctaatcgtttttcggaggtttttcagTGGAAGACGTCATCG
AAGAGACACGTGAAGAGAAAACATATAGAAATTGGATGAATTCAATGGGTGTTGAT
CCATATGTTAATTGGTTGTATGCTGATCTTCAAAATGGTGTCGTCATTTTCCAGgtaaaa
attggatgaaaaccccgagttttccggcgaaaaatggacaaaattccggaaaaaccgattttcccggcggaaatttgacaaaaatcgaaaaa
aatctaattttccggcggaagtttaacaaaattccggataaatcgatatttccggcggaaaatcaataaaaatccggaaaaattgcattttccgg
cgaaaaatggacaaaaatattcagaaaaattgcaaaagttgagtttttcgaggtattttagctcaaaacggatttatctaaattttttactgaaattt
cgcgaaattttgacttttttgaagatttttcacattttttgtgaaaaatctgcaaattattcgatttttgctctgaaaatttcagtttttgagttttccaaga
atttcagccgaaaaacggattttttcggtttttctgaaatttctgtggaaaatcaaaattttcaagacctttgtgtaatttttcagcccgcatttttacc
gagaaatcggcggaagtttaacaaaattccggaaaaatcgatatttccggcggaaaatcaatggaaatccggaaaaattgcattttccggcg
aaaaatggacaaaagttcaggaaaaatcaattttgccctgcgaaaaatcaataaattcagaaaaacctcattttaacggcgaaatttcggcaa
aattcggtgtgaatttgccctaaaaaaagatctaaaacttgccaattttttcggaaaatccgtatttttgtaccaaaaattcaaatttcacagttaca
gcatgtgcgcttcattgagaaaatcactcaaaattcaaattttgaagattttctcaatggggcgcactttcaatcgatgcgatgtttaaattttttaa
attttcagtcaaaaaaaaatgccaattttgcgagtttttgcagaaaattcgcagtttttccgcatttttcgctataattttcaaaaaaagaatttcagC
TTTACGATATAATCCGACCGGGAATGGTAACATGGAAACGAGTTGTACGGACATTCC
ATAAGCTTCGAGGAATGATGGATCAAATTCAGAATTGCAATTATGCTGTTGAACTTG
GAAAACAGCTCAGATTCTCACTTGTCGGAATTCAGGGAAAAGgttgatacttaaaattttttggaattt
ttacggaaaattatgacaaaaatttacgaaacccatgaaaatttggctgatttttggtctgaaaatgctgaaatcggcagatttttgactaaagaa
tttgctgaaaacggagaattcttttttttttgttgaaaatcgctaaaaatttaataatttccgttttttttggtgaaaaatcttaattttttgactgaaaatt
gtctggattgatcaactgtaaatttgtgaaaaactggaaaaatcgaaaaaaaattgatttttaaatgaaaatcttgaatttttccgtaagttctgtatt
aaaacacgaaaaatttcaatttttctccattttgcagctaaaaatctgaaaattttctaaaaatcaaattttatagtagatacttaaaattttttggaatt
tttacggaaaattatgacaaaaattcgaaacccatgaaaatttggctgatttttggtctaaaatgctgaaatcggcagatttttgactaaagaattt
gctgaaaacggagaattatattcgctttataaatttttattttccgaaataaaaatcagcaaatcgaaattttcatacaacccgccgactcaaaaa
aaaacacaaaaatttatggttttcatttaaattttccaacaaaaaaaaacccaaaaaaatctaattttccacacaaaagttccctaaaaaccttcca
aactctaatgttatcacaaaaacccaaaaacttgggtgaaattcattttcattcagattttcctttttttaaattcctaaaaacctaatttttcacgcaa
aaaactagacattttccaaaaaatattaattttccccccgaaaatctaaaaagttcccaaaaaattcaaatttccaccctaaaatccaaaaatttcc
atcctaaaaccaaaatttttccaaaaaaaaaaatcgtatttttctccaaatttacccaatttttcttggaaaacagctcagattctcacttgtcggaat
tcagggaaaaggtagagagaaattcttcagaaataccaagaatttggctgaaaactggacaaattttgagatttttggcgtaactaactgtaaa
aagcgtgaaattttgaaagagaaatctggaaaaaatcataaaaatgattaggaaattactgaaaattataattttttcaaattgagatagatattttt
tgctgaaataacgaaaaatatgtgcttttattgaaaaaaaataccaaaaaaaacatgaagatatggaattttccaaaaaaaaaaaaatccacttt
ttcagagaaaaaattgaatatttttacaaatttcacccaaaaaaccgataaaatgccataaaaaagagcacaattttcaattttaggaaatttcata
tttttgaaataaaaaatcgcttaaaaatcctttttttctgaaaatgctcaaaattcgcaaaagagctcagcttgctccaaattatgctcgaaaaaac
agaaaaatctcagaaatttgctacgaaaagctctgaaaacttcaaaattgccaaaattgccgaaaatgggccaaaattttgatgagaacgaac
ttttttgacaaaaaaaaatttgtccaaaaaatcgcatttttctccaaatttaccaacatttttcgttacagATATCTACGACGGCAAT
CAAACATTGACATTAGCTCTCGTTTGGCAGCTAATGCGTGCCTATACCCTCTCAGTTC
TTGCTCAATGTACTCAATCTGGTGATTCCCTGCCAGCTGATAAGGATATTGTTGCATG
GGTTAACGAAAAGCTCAAAAATTCTGGAAAATCCACATCAATTCGATCGTTCCAAG
ACCCGGCAATTTCCGATGGAAAAGTTGTATTGGATCTGATTGATGCAATTAAACCAA
ACGTTATTGATCATAGTTTGGTGAAAAGTGGCAAAAGTAATGAGGATAAAATGTCA
AATGCAAAATATGCAATCACTTGTGGACGAAAGATTGGAGCAAAGATTTACGCGTT
GCCAGAGGATATTGTTGAGgttaatattaccccgaaaaattgaagaaaaacggagaaaaatgactgaaaatttgaaattttc
agttgaaatttaatcaaaaacgtggaaattttaagaaaaatgactgaaaatcatgaaaaatcaatgatttcggaatttgaagcaaaaaaattcca
gaagttggagattttcggctaaaatctcactaaaaacttgaaaaattggagaaaaacggagaaaaatgactgaaaattcgagattttggatgg
aaatttgtttttttcccggcaaatacggctaattttcaattciggcaatttgctgatttgccggatttttttttggcaaattggcgatttgccggtaatat
ttattttctggcagattgcccatttgccgggaatttttattttctgacaaattgccggtttgccggaaatttttatttcctggcaaattgccgatttgcc
ggaaatttttattctctagcaaattggcgatttgccggaaatatttattttctggcagattgcccatttgccgggaatttttattttctgacaaattgcc
catttgccggaaatttttatttcctggcaaattgccggtttgccggaaatatttattttctggcagattggcgatttgccgggaatttttattttctgag
aaattgccgatttgacggaaattcttattttctgagaaattgcccatttgccgggaatttttattttctgacaaattgccgatttgccggaaatttttat
atcaaaatgattgtaaaacacataatttccagaaaatgtttgaaaaatctacaattttgctctaaaatctcaaaaaaaaatgctccgaaaagctct
gaaaatcgtccaaaaccccaaattgttttcagGTGAAGCCAAAAATGGTGTTGACAGTGTTTGCATGTCT
AATGGCTCGTGACTATCTTCCCGATATGAAGCAAGGAGCAGCTTCTGCTCCAATTGT
TCCGATGATTAATGGAAATTAGttctgcaaaaatttcgaatttcttgaatttcccgccgaaaaaaaatcgcggaaatacaa
attttcctccatatatatatctctattttcccgtttttccatcttttttctcacaaaaattgcgtcccgtaagctttaaatgtgatatattcaatttctcccca
ctcattgccacgtggcaaatcacttttttccaattttttttcactttaattgctcatttgctcctccgtttgttactgatttttttctattgcatttaacccctc
ccgtatttttttcgtgtatttctactaattttcgccttttttctcggatttttggtggaaaattggcggttttttttgaatattttttacccgaaaaatcgagtt
tctagcctaaatttggaaaaaattgagtttttcaaatttttggaccctttttacgctgaaaaattgcaaaagttgagtttttcgaggtattttagctcaa
aacggatttatctaaattttttactgaaatttcgcgaaattttgacttttttgaagatttttcacatttttggtgaaaaatctgcaaatttttcgatttttgct
cttaaaatttcagtttttgagttttccaagaatttcagccaaaaaacggattttttcggtttttctgaaatttctctggaaaatcaaaatttctttcaaga
cctttctgtaatttttcagcccgcatttttaccgagaaatcggcaatttttccgatatttcaaatcgaaatttgaataattatcggatttttttactccaa
aaatctaattcggattttggtagaaatttcgaaaaaatcccaaaaaaaatattttttaaaaaaatcccgaaaactcgccgctttttctcccaaaatc
cgaaatctaacactctgtgaacaactcgtcaaaaattcaaaattatctcttctaatctctcttatctcactcttctcatttttccagttatttgaccaattt
ccccattgctgctgagcttcttcaatccgagccggcaccccccaacaaattcactgtgaattcccgaccggttgttcgaagcttttcccaacctt
cclgttatccatattattattattattcagalgaattttccaaaaattttaatagtttttcggcttttgcgctgccaataaaatttcaaa
      ● (plastin-1A) [C. elegans] - SEQ ID NO: 44
gcgacggtgaaactcttgaagatctccggcgtctgtccccagaagaaattctcATGAGATGGGTTAATTATCATTTG
GAACGTGCTGGAACTCAACGTCGCCTACACAATTTCACATCAGATATTGTCGATTCA
GAGATTTATACTCATTTGCTCCATCAAATTGCTCCAAATGGCTCCGGTGTCACGTTAT
CTCCACTCGGAGTCCATGGAAATGTCCCAAGAGCCGGAGCAATGTTAGATGAAGCC
GAGAAGCTTGATTGTCGGGAATTTGTGACAGCAACTGATGTGGCAGCCGGAAATTA
CAAATTAAACTTGGCTTTTGTTGCCAATTTGTTCAATAAACATCCAAATTTGCCGGAT
CCGGGAGCGGATGAAGTTGgtgagtttttgaaaaagtcgaaaattgactgaaaaataaaaaatttcaaaatttcaaaatgac
aacttccagcgattttaattaaaaaaggtccgataaaaaaaaaaaaataagatttttcatcgaaaattcaccgactcaccgaagt
      ● (plastin-1B) [C. elegans] - SEQ ID NO: 45
atgccaattttgcgagtttttgcagaaaattcgcagtttttccgcatttttcgctataattttcaaaaaaagaatttcagCTTTACGATAT
AATCCGACCGGGAATGGTAACATGGAAACGAGTTGTACGGACATTCCATAAGCTTC
GAGGAATGATGGATCAAATTCAGAATTGCAATTATGCTGTTGAACTTGGAAAACAG
CTCAGATTCTCACTTGTCGGAATTCAGGGAAAAGgttgatacttaaaattttttggaatttttacggaaaattat
gacaaaaatttacgaaacccatgaaaatttggctgatttttggtctgaaaatgctgaaatcggcagatttttgactaaagaatttgctgaaaacg
gagaattcttttttttttgttgaaaatcgctaaaaatttaataatttccgttttttttggtgaaaaatcttaattttttgactgaaaattgtctggattgatca
actgtaaatttgtgaaaaactggaaaaatcga
      ● E3 ubiquitin-protein ligase (siah-1) [C. elegans] GenBank ID: 177138
                o Available on the world wide web at
                          ▪ ncbi.nlm.nih.gov/gene/177138
                          ▪ ncbi.nlm.nih.gov/nuccore/NM_068008.6
                o Protein Sequence - SEQ ID NO: 46
MSNRNGGGGGGDYQDVIESLRRTQLIFEEDDNNSSDSAPQANHLSRHQTSNSLVEDMV
NHSNGNPPPVPPGITQQQCQIGLTPRMSASPPSAVSTISGTAVLGKTMARVQSNPPGSIPH
NTTTTAQGIQSVAPHIPIGGGGATDDSSAEILSVFECPVCLEYMLPPYMQCSSGHLVCSN
CRPKLQCCPTCRGPTPSVRNLGLEKIANTVRFPCKFSTSGCPLNFHHADKTEHEELCEFR
PYCCPCPGASCKWQGGLSDVMEHLKKIHKSITTLQGEDIVFLATDINLPGAVDWVMMQ
SCFDYNFMLVLEKQEKYDPAQPTQMFYAVVQLIGSKKEADNFVYRLELSASRRRMSWE
ATPRSIHEGVVVAIQQSDCLAFDSNAAQLFAENGNLGINVTISRIDTPQRRHPNELENSDL
EYD
                o Nucleotide Sequence - SEQ ID NO: 47
agacgacggg aagacggcgg catccgcaga caccgaacca cccaccacct ccaacatcgt cgtcctcgtc atctcgactg
ccttcgctga tgagtaatcg gaacggtggt ggaggaggag gagactatca ggacgtgatc gagtctcttc gtcgtacaca
gctcatcttt gaggaggacg ataataatag tagcgattcg gctccgcaag cgaatcatct atccagacat cagactagca
attctttagt cgaagatatg gttaatcact caaatggcaa ccctccaccg gtcccgccag gcatcacaca acagcaatgt
caaatcggat tgacacctcg gatgtcggca tcgccaccgt ctgcagtgtc aacaatctct ggaaccgcag tgcttggcaa
aacgatggcc agagttcaga gtaatccccc gggaagtata ccgcacaaca caacgacgac cgcgcaagga atccaaagcg
tcgcaccaca tattccaatc ggcggcggtg gagccaccga cgacagcagt gcagagatct tgtcggtgtt tgagtgcccg
gtatgcctgg agtacatgct gccaccatac atgcagtgct catccggaca tcttgtctgc tcgaactgca gaccaaagct
tcagtgctgc ccgacttgtc ggggtccaac tccttctgtc cgcaacctgg gcctcgagaa gatcgccaac accgtccgct
ttccatgcaa attctctacg tccggctgcc ccctgaactt ccatcacgct gataagaccg agcacgagga gctctgcgaa
ttccgaccct actgctgccc gtgccctgga gctagctgca agtggcaggg aggcttgtcg gatgtgatgg agcatttgaa
gaagatccac aagagcatca cgacgcttca aggcgaggat atcgtcttcc tggccactga catcaatctt cctggagcag
ttgactgggt tatgatgcag agctgctttg actataattt tatgttggtg ctcgagaagc aggaaaagta cgatccagct cagccaactc
agatgttcta cgcagtcgtt cagctgattg gctcgaagaa ggaagctgat aactttgtgt accggcttga gctatcggcg
agccgccgga gaatgtcatg ggaagccaca ccgagaagca tccacgaggg ggggtagtg gccattcaac agtcggattg
cctagcgttc gattcaaatg ctgcacagct ttttgcagaa aacggaaacc ttggaatcaa cgtcactatt agccgaatag
atactccaca gaggcgccac ccaaatgagt tggaaaattc tgatctcgag tacgactaga tatgcacagt gtaaacgtaa
taaaattatt tttgattttt ttcttcaaaa atgtgatgaa aagcttaaaa aattcagttg aaaatcccct tacaatcaga acattttaaa
aaatttaaaa attaaaaaaa ttttttt
      ● Serine/threonine-protein kinase (unc-51) [C. elegans] GenBank ID: 180311
                o Available on the world wide web at
                          ▪ ncbi.nlm.nih.gov/gene/180311
                          ▪ ncbi.nlm.nih.gov/nuccore/NM_075468.7
                o Protein Sequence - SEQ ID NO: 48
MEQFDGFEYSKRDLLGHGAFAIVYRGRYVDRTDVPVAIKAIAKKNISKSKNLLTKEIKIL
KELSSLKHENLVGLLKCTETPTHVYLVMEFCNGGDLADYLQQKTTLNEDTIQHFVVQIA
HALEAINKKGIVHRDLKPQNILLCNNSRTQNPHFTDIVIKLADFGFARFLNDGVMAATLC
GSPMYMAPEVIMSMQYDAKADLWSIGTILFQCLTGKAPFVAQTPPQLKAYYEKTRELR
PNIPEWCSPNLRDLLLRLLKRNAKDRISFEDFFNHPFLTSPLLPSPSKRILESARSPLLANR
RIITPQSSLPVPKRAGSTKLDSPTPVRRIGESPRVQRRVITPGMPSPVPGAPMQESTDFTFL
PPRQESSPVKQVQVHTNVSPSLTTCKPVPVPSQRLTYQKMEERLAAARKTAVPSSSSPTG
SAVSAQHQHQHQQQQEPASSPVVQRIERPDQLPRRTTLQDPNAHDIERMTMPNPTFVVC
GSSTKPSPNNANRVRRSTITSPADTQDMVAADQMLSNLDPTTTTTTIPKSATTANIQGIPR
GARDRSVTSPPQPTIHENEPLDNAKYQQTDVNNSPTAPTEPFIIKNQTTCSTSSTSSSVVEE
EEAMSLPFASGSHLAAGFKKTPAEVPMDHGALPPALDQEIVLGEEHKQILAKLRFVAEL
VDTLIHVAEQKDNPLASAMASRRQLLTTGTSTTNTSSPYRRAEQLVVYVRALHMLSSAL
LLAQTNVANRVLHPSVAVQQVLNQLNDKYHQCLVRSQELASLGLPGQDPAMAVISAER
IMYRHAIELCQAAALDELFGNPQLCSQRYQTAYMMLHTLAEQVNCDQDKTVLTRYKV
AVEKRLRILERQGFVAAVNT
                o Nucleotide Sequence - SEQ ID NO: 49
aacctaccta caaaaccccc cgccaaaacc tatatagtca cacaataatc acccaaccca gctgaatgga gcagtttgac
ggcttcgagt acagcaaacg ggacctttta ggtcatggag catttgcaat tgtatacaga ggacgctatg ttgatcgcac
agacgtgcca gttgccatca aggcgatcgc caagaagaat atcagcaaat caaagaatct gctgacaaaa gagattaaaa
ttctaaaaga attgtcaagc ctgaagcatg aaaatcttgt gggcctactc aaatgcacgg agacccctac tcatgtgtat ttggttatgg
aattctgtaa tggaggagat ttggctgatt atttacaaca gaagactaca ttgaatgagg atactattca acattttgtc gtgcaaattg
ctcacgcgtt ggaagcgatc aacaaaaagg gcatcgtaca tcgtgatttg aagccccaaa acatcctcct ctgcaacaac
tcccgcaccc aaaatcccca cttcaccgac atcgtcatca agcttgccga ctttggattc gcccgattcc tcaacgacgg
tgtgatggcg gcaactctct gcggatcccc aatgtacatg gctcccgaag tcataatgtc aatgcaatac gacgcgaaag
ccgatctctg gtcaattgga accatcctct tccagtgtct caccggaaaa gccccattcg tcgcgcagac cccgccgcag
ttgaaggcct attacgagaa gactcgcgag ctgagaccca atattccaga gtggtgctct ccgaatctac gggatcttct
gttgagactg ctcaagagaa acgccaagga tcggatctcg ttcgaagact tcttcaatca cccgttcctc acctcacctc
tccttccgtc gccgtcgaag agaatcctcg agagcgcccg atcaccacta ctggcgaatc gccgaatcat cacaccacaa
tcttcgcttc cagtacccaa gcgagccgga tccaccaagc tcgacagccc gacgccagtt cggaggatcg gagagtcacc
acgtgtacag agaagagtca tcacacccgg aatgccgtcc ccagtgccgg gagctccaat gcaagagtcc actgatttca
ctttccttcc accaaggcag gaatccagtc cggttaagca ggtccaagtg cataccaatg tctcaccatc actaacaact
tgcaagccgg tcccagtgcc gagtcaacgt ttgacctacc agaaaatgga ggagcgtctg gcggctgcaa gaaagactgc
agtgccttct tcttcttctc caacaggatc agcggtttct gctcagcatc agcatcagca tcagcagcag caggagcccg
cgtcgtcgcc ggtggttcag cggattgaaa ggcccgatca gctgccaaga cggactacgc ttcaggatcc gaatgctcat
gacattgaga ggatgactat gccgaatccg accttcgtcg tatgtggcag cagcacaaaa ccatcgccga acaacgcgaa
tcgcgttcgc cgctccacca tcacaagccc cgccgacacc caggatatgg tcgctgctga ccaaatgctt tcgaacctag
accccaccac caccaccacc accatcccca aatcggccac cacagccaac atccagggaa ttccacgtgg cgctcgcgat
cgatcggtca cttcgccgcc gcagccgacg attcatgaga atgagccgtt ggataatgca aagtatcagc agacggatgt
taataattca ccgacagcac ccacggagcc atttattatt aagaatcaga caacatgctc cacatcctca acctcctcct
cagtcgtcga agaggaagag gcgatgagtc ttccattcgc gtctggaagc catctcgcag caggattcaa gaagaccccc
gccgaggttc caatggatca cggagcccta ccaccagctt tggatcagga aattgtgctc ggcgaggagc acaagcagat
tctcgcgaag cttcgatttg tcgccgagct cgtcgatacc cttatccacg tggccgagca gaaggacaat ccgttggcgt
cggcgatggc ttcaaggaga cagcttttga ctactggaac ctcaacgact aatacatcgt caccgtaccg gagagccgag
cagctggtgg tgtacgtcag agctctgcac atgctctcct cagcactgct cttggctcag actaacgtgg caaatcgggt
acttcatccg agtgtcgccg tgcagcaggt gctcaatcag ctcaacgaca agtatcatca gtgtttggtc cgctcccagg
agcttgcctc gcttggcctc cccggacagg atccggcgat ggcggttatc tcggcggaaa gaatcatgta cagacatgcg
atagaactgt gccaggctgc tgctcttgac gagctttttg gaaaccccca gctgtgcagt cagaggtacc agaccgccta
catgatgctt cacaccttgg ccgagcaggt caattgcgat caggataaga cggtgctcac gaggtacaaa gtcgctgtgg
aaaaacgtct tcgaattctg gaacgacagg gatttgtggc agctgtgaac acataatagt ccttttaccc ccacttttgt tctgtttttt
gtgctgcttc ttcccccatt tctaccccgc caaaatgagc ctctcccttg caatgaacgt gtagcccccc actctattga aaaatgagat
tttcactacc ctaaacttct tagaattctt ttttctattg ttttaatgag attttttttt tgaaaagacc tgtaaatccc gttccggaac
tagtcgaaca taatgcatca ttcgtgaata aattttgttg tacagtt
```

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A composition which modulates at least one of mitophagy mitochondrial stress level, and mtDNA heteroplasmy level having efficacy for the treatment of mitochondrial disease, comprising effective amounts of one or more agents selected from hemin, tripterin, folinic acid, lithium chloride, metformin, N-acetylcysteine, nicotinamide, resveratrol, valproic acid, dexamethasone, etoposide, vorinostat, quercitin, hydralazine, thiamine, lipoic acid, pfithrin-alpha, ginsenoside, sulfonsuccinimidyl oleate, carnitine, AICAR, GSK2578215A, an inhibitory nucleic acid and an activating genetic construct targeting a mitophagy modulator protein encoding nucleic acid in a pharmaceutically acceptable formulation.

2. The composition of claim 1, which increases mitophagy or mitochondrial stress level or decreases mitophagy or mitochondrial stress level.

3. (canceled)

4. The composition of claim 1, which increases mtDNA heteroplasmy level, or decreases mtDNA heteroplasmy level.

5. The composition of claim 1, wherein at least two agents are present and are administered separately or said at least two agents are present and are administered together.

6.-7. (canceled)

8. The composition of claim 5, wherein said at least two agents are selected from one or more of vorinostat, hemin, GSK2578215A, tripterin and resveratrol.

9. The composition of claim 1, wherein said at least two agents are a combination of hemin and tripterin or a combination of thiamine and tripterin, which act synergistically to modulate one or more of mitophagy, mitochondrial stress level, and mtDNA heteroplasmy level and are administered separately or together.

10. A method for alleviating symptoms associated with mitochondrial disease, comprising administration of the composition of claim 1 to a patient in need thereof.

11. The method of claim 10, wherein said symptoms include one or more of muscle weakness, exercise intolerance, chronic fatigue, gastrointestinal dysmotility, impaired balance, peripheral neuropathy, metabolic strokes, dysautonomia, vision loss, eye muscle and eyelid weakness, hearing loss, glomerular or tubular renal disease, endocrine dysfunction, diabetes mellitus, dyslipidemia, cardiomyopathy, arrhythmia, anemia, failure to thrive, over or underweight, developmental delay, neurodevelopmental regression, cognitive decline and memory impairment, migraines, headaches, Parkinsonism, dystonia, liver dysfunction or failure, infertility, metabolic instability, stressor-induced acute decompensation DLD disease, Mitophagy disorders, Mitochondrial lipid biogenesis disorders, mitochondrial cofactor disorders, and secondary mitochondrial disorders including but not limited to resulting from toxins, drugs, age, prescribed or illicit medications, smoking, alcohol, environmental exposures, obesity, aging, and genetic disorders that secondarily impair mitochondrial function, structure, or activities.

12. The method of claim 10, wherein said mitochondrial disease is selected from the group consisting of Complex I disease, Complex II disease, Complex III disease, Complex IV disease, Complex V disease, Multiple respiratory chain complex disease, adenine nucleotide translocase deficiency, pyruvate dehydrogenase deficiency, mitochondrial depletion disease, multiple mitochondrial DNA deletions disease, mitochondrial DNA maintenance defects, mitochondrial translation defects, mitochondrial nucleotide import disease, mitophagy disorders, Friedreich's ataxia, Leber's Hereditary Optic Neuropathy, Kearns-Sayre Syndrome, Pearson Syndrome, Chronic Progressive External Ophthalmoplegia, Autosomal Dominant Optic Atrophy, Mitochondrial Myopathy, Mitochondrial Encephalomyopathy with Lactic Acidosis and Stroke-Like Episodes, Myoclonic Epilepsy and Ragged Red Fibers Syndrome, Neurogenic Ataxia and Retinitis Pigmentosa, Mitochondrial Neuro-Gastrointestinal Encephalomopathy, maternally inherited diabetes and deafness, primary lactic acidosis, Leigh syndrome, Leigh-like syndrome, and multi-system mitochondrial disease.

13. The composition of claim 1, wherein said agent is
i) an inhibitory nucleic acid or pharmacologic compound which reduces expression of one or more nucleic acids encoding a mitophagy modulator protein selected from atg-9, dct-1, pink-1, sqst-1, hrdl-1, mul-1, pdr-1, plastin-1, siah-1, unc-51, and uaDf5 or
ii) a genetic construct or compound which increases expression of a mitophagy modulator protein selected from atg-9, dct-1, pink-1, sqst-1, hrdl-1, mul-1, pdr-1, plastin-1, siah-1, unc-51, and uaDf5 and has a sequence listed in Table 1.

14. A screening method for identifying agents which modulate mitophagy, comprising;
a) providing genetically altered C. elegans, said genetic alteration impacting a gene associated with mitophagy, and wild-type C. elegans, lacking said genetic alteration;
b) contacting the C. elegans from step a) with an agent;
c) determining whether said agent alters a cellular parameter associated with mitophagy pathway activity(s) in C. elegans comprising said genetic alteration relative to wild type C. elegans; agents which alter said parameter in said genetically altered C. elegans being identified as modulators of mitophagy wherein said C. elegans is optionally contacted with a stressor prior before, after, or concomitantly with said agent.

15. The method of claim 14, wherein said cellular parameter is selected from the group consisting of fecundity, egg hatching rate, development, lifespan, stressor survival, healthspan, animal activity, swimming capacity, thrashing activity, pharyngeal pumping rate, mitochondrial oxidant burden, cellular oxidant burden, antioxidant capacity, glutathione levels, reduced (GSH) to oxidized (GSSG) glutathione ratio, CI enzyme activity, CI enzyme assembly, CII enzyme enzyme activity, CIII enzyme activity, CIV enzyme activity, complex V enzyme activity, oxygen consumption capacity, ATP production, ATP levels, nicotinamide adenine dinucleotide (NADH and NAD⁺) levels, (NADH and NAD⁺) ratio, NAD metabolism, mitochondrial membrane potential, mitochondrial content, mitochondrial structure, mitochondrial ultrastructure, mitochondrial unfolded protein response, mitochondrial import, mitophagy, autophagy, cytosolic translation activity, nutrient-sensing signaling profile, unfolded protein response activation, lysosomal number, lysosomal activity, lysosomal pH, proteasome number or activity, transcriptome-wide signaling, transcription factor signaling, kinase signaling, amino acid pathway profiles, intermediary metabolic flux dynamics or rates, steady state metabolism of intermediary metabolites, amino acid levels, organic acid levels, ammonia levels, and glycoprotein production, cellular proliferation, cell growth, lactic acid level, glycolysis, cellular redox levels, and lactate/pyruvate ratio.

16. The method of claim 14, wherein said C. elegans comprises a mutation in a gene that modulates one or more of mitochondrial structure, content, biogenesis, proliferation, destruction, and function or said C. elegans is genetically altered via introduction of a silencing RNA or antisense oligonucleotide that targets a gene that modulates mitophagy, or said genetic alteration is introduced into a gene that Modulates mitophagy via CRISPR-CAS, a base editor system or a TAL effector or TALEN based system.

17.-18. (canceled)

19. The method of claim 14, further comprising contacting a zebrafish comprising a mutation in the cognate zebrafish gene with said identified agent and determining whether said agent alters a cellular parameter associated with aberrant mitophagy pathway activity in said zebrafish and, or contacting a human fibroblast, lymphoblastoid cell line, myoblast cell line, myotube cell line, transmitochondrial cybrid cell line, gastrointestinal cell line, conjunctival derived cell line, cancer cell line, HEK293 cells, HELA cells, derived iPSC or a differentially terminated cell line comprising a mutation in the cognate human gene with said identified agent and determining whether said agent alters a cellular parameter associated with aberrant mitophagy in said human fibroblast or other cell line type.

20.-21. (canceled)

22. The method of claim 14, wherein said gene encodes OPA1 mitochondrial dynamin like GTPase or is a single large-scale mitochondrial DNA deletion (SLSMD) that causes SLSMD syndromes (SLSMDS) or a point mutation in mitochondrial DNA that causes a primary mitochondrial disease that may affect any organ function.

23.-29. (canceled)

30. A preclinical method for identifying mitochondrial disease subjects likely to respond to treatment for aberrant mitophagy, comprising:
a) contacting patient cell lines or cells obtained from a subject with at least one agent which modulates mitophagy as claimed in claim 1, said subject having a predetermined genotype;
b) culturing said cells under normal and stressed growth conditions, wherein said stressor is applied in increasing concentrations;
c) determining the protective effects of said agent on said cells, agents having protective action being effective to modulate mitophagy in subjects having said predetermined genotype.

31. The method of claim 30, wherein said agent activates mitophagy or reduces mitophagy and comprises one more agents listed in Table 1.

32. (canceled)

33. The method of claim 30, wherein tolerability and efficacy of said agent are assessed in a whole animal model of mitochondrial disease associated with aberrant levels of mitophagy and wherein said protective effects include one or more of improvement in cell viability, cell proliferation, ATP production, mitochondrial stress, mitochondrial unfolded protein response, mitochondrial membrane potential, mitochondrial oxygen consumption capacity, mitochondrial coupling of oxidation to phosphorylation, mitochondrial electron transport chain enzyme activities, mitochondrial metabolite levels, mitochondrial mass or content, mitochondrial oxidant levels, mitochondrial DNA mutations or oxidative stress, total cellular oxidant levels, cellular antioxidant defense enzymes, glutathione metabolism, cellular pH, and cellular oxygen capacity consumption.

34.-37. (canceled)

38. A humanized C. elegans strain expressing a mutated OPA1 mitophagy modulator protein, said mutation being selected from eat-3(R289Q) or eat-3(V328I).

39. (canceled)
